SEQUENCE LISTING (16 sequences)

SEQ ID NO: 1 - protein, 495 aa, synthetic construct
  source 1..495: note = DENV 1/3 EDII small 438-2 E chimera
MRCVGIGNRDFVEGLSGATWVDVVLEHGSCVTTMAKDKPTLDIELLKTEVTNLATLRKLCIEGKITNITTDSRCPTQGEAVLPEEQDQNYVCKHTYVDRGWGNGCGLFGKGSLVTCAKFQCLEPIEGKVVQYENLKYSVIVTVHTGDQHQVGNETTEHGTIATITPQAPTSEIQLTDYGALTLDCSPRTGLDFNEMILLTMKNKAWMVHRQWFFDLPLPWTSGATTETPTWNRKELLVTFKNAHAKKQEVVVLGSQEGAMHTALTGATEIQNSGTTTIFAGHLKCRLKMDKLTLKGMSYVMCTGSFKLEKEVAETQHGTVLVQIKYEGTDAPCKIPFSTQDEKGVTQNGRLITANPIVTDKEKPVNIEAEPPFGESYIVIGAGEKALKLSWFKKGSSIGKMFEATARGARRMAILGDTAWDFGSIGGVFTSVGKLVHQIFGTAYGVLFSGVSWTMKIGIGVLLTWLGLNSRSTSLSMTCIAVGLVTLYLGVMVQA

SEQ ID NO: 2 - protein, 495 aa, synthetic construct
  source 1..495: note = DENV 1/3 EDII large 438-3 E chimera
MRCVGIGNRDFVEGLSGATWVDVVLEHGSCVTTMAKDKPTLDIELLKTEATQLATLRKLCIEGKITNITTDSRCPTQGEAVLPEEQDQNYVCKHTYVDRGWGNGCGLFGKGSLVTCAKFQCLEPIEGKVVQYENLKYSVIVTVHTGDQHQVGNETTEHGTIATITPQAPTSEIQLTDYGALTLDCSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLTLKGMSYVMCTGSFKLEKEVAETQHGTVLVQIKYEGTDAPCKIPFSTQDEKGVTQNGRLITANPIVTDKEKPVNIEAEPPFGESYIVIGAGEKALKLSWFKKGSSIGKMFEATARGARRMAILGDTAWDFGSIGGVFTSVGKLVHQIFGTAYGVLFSGVSWTMKIGIGVLLTWLGLNSRSTSLSMTCIAVGLVTLYLGVMVQA

SEQ ID NO: 3 - protein, 495 aa, synthetic construct
  source 1..495: note = DENV 1/3 EDIII E chimera
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTIATITPQAPTSEIQLTDYGALTLDCSPRTGLDFNEMVLLTMKKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYAMCTNTFVLKKEVSETQHGTILIKVEYKGEDAPCKIPFSTEDGQGKAHNGRLITANPVVTKKEEPVNIEAEPPFGESNIVIGIGDNQHTGGRFKKGSSIGKMFEATARGARRMAILGDTAWDFGSVGGVLNSLGKMVHQIFGSAYTALFSGVSWVMKIGIGVLLTWIGLNSKNTSMSFSCIAIGIITLYLGPYFAT

SEQ ID NO: 4 - protein, 495 aa, synthetic construct
  source 1..495: note = DENV 4/2 EDI E chimera
MRCIGISNRDFVEGVSGGSWVDIVLEHGSCVTTMAQNKPTLDFELIKTEAKEVALLRTYCIEASISNITTATRCPTQGEPYLKEEQDQQYICRRDVVDRGWGNGCGLFGKGGVVTCAKFSCSGKITGNLVQPENLEYTIVVTPHSGEEHAVGNDTGKHGKEIKVTPQSSITEAELTGYGTVTMECSPRTGLDFNEMILMKMKKKTWLVHKQWFLDLPLPWTAGADTSEVHWNYKERMVTFKVPHAKRQDVTVLGSQEGAMHSALAGATEVDSGDGNHLFAGHLKCRLRMDKLQLKGMSYTMCSGKFKIDKEMAETQHGTTVVKVKYEGAGAPCKVPIEIRDVNKEKVVGRVISSTPLAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQA

SEQ ID NO: 5 - protein, 495 aa, synthetic construct
  source 1..495: note = DENV 4/2 EDII E chimera
MRCVGVGNRDFVEGVSGGAWVDLVLEHGGCVTTMAQGKPTLDFELIKTEAKQPATLRKYCIEAKLTNTTTESRCPTQGEPSLNEEQDKRFVCKHSMVDRGWGNGCGLFGKGGIVTCAMFTCKKNMEGKVVQPENLEYTVVVTVHNGDTHAVGNDTSNHGVTATITPRSPSVEVKLPGYGELTLDCEPRSGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCKVRMEKLRIKGMSYTMCSGKFSIDKEMAETQHGTTVVKVKYEGAGAPCKVPIEIRDVNKEKVVGRVISSTPLAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQA

SEQ ID NO: 6 - protein, 495 aa, synthetic construct
  source 1..495: note = DENV 4/2 EDIII E chimera
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYSMCTGKFKVVKEIAETQHGTIVIRVQYEGDGSPCKIPFEIMDLEKRHVLGRLITVNPIVTEKDSPVNIEAEPPFGDSYIIIGVDPGQLKLNWFKKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQA

SEQ ID NO: 7 - protein, 166 aa, synthetic construct
  source 1..166: note = DENV 1/3 EDII prM chimera
FHLTSRDGEPRMIVGKNERGKSLLFKTASGINMCTLIAMDLGEMCDDTLTYKCPHITEVEPEDIDCWCNLTSTWVTYGTCNQAGEHRRDKRSVALAPHVGMGLDTRTQTWMSSEGAWKQIQKVETWALRHPGFTVIALFLAHAIGTSITQKGIIFILLMLVTPSMA

SEQ ID NO: 8 - protein, 166 aa, Dengue virus
FHLTTRGGEPHMIVSKQERGKSLLFKTSAGVNMCTLIAMDLGELCEDTMTYKCPRITETEPDDVDCWCNATETWVTYGTCSQTGEHRRDKRSVALAPHVGLGLETRTETWMSSEGAWKQIQKVETWALRHPGFTVIALFLAHAIGTSITQKGIIFILLMLVTPSMA

SEQ ID NO: 9 - protein, 166 aa, Dengue virus
FHLSTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYG

SEQ ID NO: 10 - protein, 166 aa, synthetic construct
  source 1..166: note = DENV4/2 EDII prM chimera
FHLTTRNGEPHMIVSRQEKGKSLLFKTEDGVNMCTLMAMDLGELCEDTITYNCPLLRQNEPEDIDCWCNSTSTWVTYGTCTTTGEHRRGKRSVALVPHVGMGLETRTETWMSSEGAWKHAQRVESWILRNPGFALLAGFMAYMIGQTGIQRTVFFVLMMLVAPSYG

SEQ ID NO: 11 - protein, 495 aa, Dengue virus
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 12 - protein, 166 aa, Dengue virus
FHLTSRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMT

SEQ ID NO: 13 - protein, 493 aa, Dengue virus
MRCVGIGNRDFVEGLSGATWVDVVLEHGGCVTTMAKNKPTLDIELQKTEATQLATLRKLCIEGKITNITTDSRCPTQGEAVLPEEQDQNYVCKHTYVDRGWGNGCGLFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGSSIGKMFEATARGARRMAILGDTAWDFGSVGGVLNSLGKMVHQIFGSAYTALFSGVSWVMKIGIGVLLTWIGLNSKNTSMSFSCIAIGIITLYLGAVVQA

SEQ ID NO: 14 - protein, 495 aa, Dengue virus
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRVISSTPLAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQA

SEQ ID NO: 15 - protein, 166 aa, Dengue virus
FHLTTRNGEPHMIVSRQEKGKSLLFKTEDGVNMCTLMAMDLGELCEDTITYNCPLLRQNEPEDIDCWCNSTSTWVTYGTCTTTGEHRREKRSVALVPHVGMGLETRTETWMSSEGAWKHAQRIETWILRHPGFTIMAAILAYTIGTTHFQRALIFILLTAVAPSMT

SEQ ID NO: 16 - protein, 495 aa, Dengue virus
MRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFTCKKNMEGKVVQPENLEYTIVVTPHSGEEHAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVDPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGIVTLYLGVMVQA

(19) United States
(12) Patent Application Publication
(10) Pub. No.: US 2025/0127871 A1
(43) Pub. Date: Apr. 24, 2025

(54) METHODS AND COMPOSITIONS FOR RECOMBINANT DENGUE VIRUSES FOR VACCINE AND DIAGNOSTIC DEVELOPMENT

(71) Applicant: The University of North Carolina at Chapel Hill, Chapel Hill, NC (US)

(72) Inventors: Ralph Baric, Haw River, NC (US); Aravinda de Silva, Chapel Hill, NC (US); Ellen Young, Hillsborough, NC (US); Jennifer Munt, Durham, NC (US); Deanna Zhu, Chapel Hill, NC (US)

(21) Appl. No.: 18/696,032

(22) PCT Filed: Sep. 29, 2022
(86) PCT No.: PCT/US2022/077232

§ 371 Date: Mar. 27, 2024

Related U.S. Application Data
(60) Provisional application No. 63/279,949, filed on Nov. 16, 2021.
(60) Provisional application No. 63/250,687, filed on Sep. 30, 2021.

(51) Int. Cl. A61K 39/12; A61K 39/00; A61P 31/14; C07K 14/005; C12N 7/00

(52) U.S. Cl. CPC A61K 39/12; A61P 31/14; C07K 14/005; C12N 7/00; A61K 2039/5258; A61K 2039/575; C12N 2770/24122; C12N 2770/24123; C12N 2770/24134

(57) ABSTRACT

The present invention provides compositions and methods of use comprising a chimeric dengue virus E glycoprotein comprising a dengue virus E glycoprotein backbone, which comprises amino acid substitutions that may introduce a domain and/or an epitope that is recognized by an antibody from a dengue virus serotype that is different from the dengue virus serotype of the dengue virus E glycoprotein backbone.

### STATEMENT OF PRIORITY

This application claims the benefit, under 35 U.S.C. § 119(e), of U.S. Provisional Application No. 63/250,687, filed on Sep. 30, 2021, and U.S. Provisional Application No. 63/279,949, filed on Nov. 16, 2021, the entire contents of each of which are incorporated by reference herein.

### STATEMENT OF GOVERNMENT SUPPORT

This invention was made with government support under Grant Number A1107731 awarded by the National Institutes of Health. The United States government has certain rights in the invention.

### STATEMENT REGARDING ELECTRONIC FILING OF A SEQUENCE LISTING

A Sequence Listing in XML format, entitled 5470-919WO_ST26.xml, 22,118 bytes in size, generated on Sep. 28, 2022 and filed herewith, is hereby incorporated by reference in its entirety for its disclosures.

### FIELD OF THE INVENTION

The present invention is directed to dengue virus vaccines that induce neutralizing antibodies against more than one dengue virus serotype from a single source.

### BACKGROUND OF THE INVENTION

Dengue is the most prevalent vector-borne virus plaguing our world. In 2010, there were an estimated 390 million infections worldwide, 25% of which were symptomatic. Properties of antibodies that correlate to protection after natural infection or vaccination have not been fully described, which hinders our ability to measure vaccine efficacy.

The present invention overcomes previous shortcomings in the art by providing chimeric dengue viruses that induce neutralizing antibodies against more than one dengue virus serotype from a single source.

### SUMMARY OF THE INVENTION

In one aspect, the present invention provides a chimeric dengue virus E glycoprotein comprising the amino acid sequence of any one of SEQ ID NOs:1-6.

In some embodiments, a chimeric dengue virus E glycoprotein of the present invention may interact with a wildtype or chimeric prM glycoprotein. In some embodiments, a prM glycoprotein of the present invention may comprise the amino acid sequence of any one of SEQ ID NOs:7-10.

Additionally provided herein is a vector (e.g., a virus vector, e.g., a nanoparticle), a dengue virus particle, a flavivirus particle and/or a virus like particle (VLP) comprising the E glycoprotein of this invention.

An isolated nucleic acid molecule encoding the E glycoprotein of this invention is also provided herein, as well as an isolated nucleic acid molecule encoding the vector, dengue virus particle, flavivirus particle or VLP of this invention.

Also provided herein is a nucleic acid molecule, vector, dengue virus particle, flavivirus particle, or VLP of this invention, further encoding and/or comprising a wildtype and/or chimeric prM glycoprotein such as provided herein.

The present invention also provides a composition comprising the E glycoprotein of this invention in a pharmaceutically acceptable carrier and also provides a composition comprising the nucleic acid molecule of this invention, the vector of this invention, the particle of this invention and/or the population of this invention, in a pharmaceutically acceptable carrier.

The present invention further provides the E glycoprotein of this invention, the chimeric prM glycoprotein of this invention, the vector of this invention, the dengue virus particle of this invention, the flavivirus particle of this invention, the VLP of this invention, the nucleic acid molecule of this invention, the vector of this invention, the population of this invention and/or the composition of this invention, singly or in any combination, for use in the manufacture of a medicament for producing an immune response to a dengue virus in a subject, for treating a dengue virus infection in a subject in need thereof, for preventing a dengue virus infection in a subject and/or for protecting a subject from the effects of dengue virus infection, and/or for use in any of the methods as disclosed herein.

Also provided herein is the use of the E glycoprotein of this invention, the chimeric prM glycoprotein of this invention, the vector of this invention, the dengue virus particle of this invention, the flavivirus particle of this invention, the VLP of this invention, the nucleic acid molecule of this invention, the vector of this invention, the population of this invention and/or the composition of this invention, singly or in any combination, for use in producing an immune response to a dengue virus in a subject, in treating a dengue virus infection in a subject in need thereof, in preventing a dengue virus infection in a subject and/or in protecting a subject from the effects of dengue virus infection, and/or for use in any of the methods as disclosed herein.

Also provided herein is a method of producing an immune response to a dengue virus in a subject (e.g., a subject in need thereof), comprising administering to the subject an effective amount of the E glycoprotein of this invention, the chimeric prM glycoprotein of this invention, the vector of this invention, the flavivirus particle of this invention, the VLP of this invention, the nucleic acid molecule of this invention, the population of this invention, and/or the composition of this invention and any combination thereof.

Additionally provided herein is a method of treating a dengue virus infection in a subject (e.g., a subject in need thereof), comprising administering to the subject an effective amount of the E glycoprotein of this invention, the chimeric prM glycoprotein of this invention, the vector of this invention, the flavivirus particle of this invention, the VLP of this invention, the nucleic acid molecule of this invention, the population of this invention, and/or the composition of this invention and any combination thereof.

Further provided herein is a method of preventing a disorder associated with dengue virus infection in a subject (e.g., a subject in need thereof), comprising administering to the subject an effective amount of the E glycoprotein of this invention, the chimeric prM glycoprotein of this invention, the vector of this invention, the flavivirus particle of this invention, the VLP of this invention, the nucleic acid molecule of this invention, the population of this invention, and/or the composition of this invention and any combination thereof.

As an additional aspect, the present invention provides a method of protecting a subject from the effects of dengue virus infection, comprising administering to the subject an effective amount of the E glycoprotein of this invention, the chimeric prM glycoprotein of this invention, the vector of this invention, the flavivirus particle of this invention, the VLP of this invention, the nucleic acid molecule of this invention, the population of this invention, and/or the composition of this invention and any combination thereof.

In further aspects, the present invention provides methods of identifying the presence of a neutralizing antibody to specific dengue virus serotypes or combinations thereof (e.g., 3/1 and/or ⅓; e.g., 2/4 and/or 4/2) in a biological sample from a subject, comprising: a) administering a composition comprising a particular E glycoprotein this invention to the subject in an amount effective to induce an antibody response to the E glycoprotein; b) contacting a biological sample from the subject with flavivirus particles comprising the particular E glycoprotein above under conditions whereby neutralization of the flavivirus particles can be detected; and c) detecting neutralization in step (b), thereby identifying the presence of a neutralizing antibody to the specific dengue virus serotypes or combinations thereof in the biological sample from the subject.

The present invention additionally provides a method of identifying the presence of a neutralizing antibody to specific dengue virus serotypes or combinations thereof (e.g., 3/1 and/or ⅓; e.g., 2/4 and/or 4/2) in a biological sample from a subject, comprising: a) contacting a biological sample from a subject that has been administered a particular E glycoprotein of this invention with flavivirus particles comprising the E glycoprotein under conditions whereby neutralization of the flavivirus particles can be detected; and b) detecting neutralization in step (a), thereby identifying the presence of a neutralizing antibody to the specific dengue virus serotypes or combinations thereof in the biological sample from the subject.

In other embodiments, the present invention provides a method of identifying an immunogenic composition that induces a neutralizing antibody to specific dengue virus serotypes or combinations thereof (e.g., 3/1 and/or ⅓; e.g., 2/4 and/or 4/2) in a subject, comprising: a) administering an immunogenic composition comprising a particular E glycoprotein of this invention to a subject in an amount effective to induce an antibody response to the E glycoprotein; b) contacting a biological sample from the subject with flavivirus particles comprising the E glycoprotein of step (a) under conditions whereby neutralization of the flavivirus particles can be detected; c) determining if the biological sample comprises an antibody that neutralizes flavivirus particles comprising the E glycoprotein of step (a); and d) identifying the immunogenic composition as inducing a neutralizing antibody to the specific dengue virus serotypes or combinations thereof in the subject if the biological sample comprises an antibody that neutralizes flavivirus particles comprising the E glycoprotein of (a).

Further provided herein is a method of identifying an immunogenic composition that induces a neutralizing antibody to specific dengue virus serotypes or combinations thereof (e.g., 3/1 and/or ⅓; e.g., 2/4 and/or 4/2) in a subject, the method comprising: a) contacting a biological sample from a subject that has been administered an immunogenic composition comprising a particular E glycoprotein of this invention with flavivirus particles comprising the E glycoprotein under conditions whereby neutralization of the flavivirus particles can be detected; b) determining if the biological sample comprises an antibody that neutralizes flavivirus particles comprising the E glycoprotein of step (a); and c)identifying the immunogenic composition as inducing a neutralizing antibody to the specific dengue virus serotypes or combinations thereof in the subject if the biological sample comprises an antibody that neutralizes flavivirus particles comprising the E glycoprotein of (a).

The present invention also provides a method of detecting an antibody to a specific dengue virus serotype or combination thereof in a sample, comprising; a) contacting the sample with a particular E glycoprotein of this invention under conditions whereby an antigen/antibody complex can form; and b) detecting formation of an antigen/antibody complex, thereby detecting an antibody to the specific dengue virus serotype or combination thereof in the sample.

Additionally provided herein is a method of identifying an antibody to a specific dengue virus serotype or combination thereof in a biological sample from a subject, comprising: a) administering a composition comprising a particular E glycoprotein of this invention to the subject in an amount effective to induce an antibody response to the E glycoprotein; b) contacting a biological sample from the subject with the E glycoprotein of (a) under conditions whereby an antigen/antibody complex can form; and c) detecting formation of an antigen/antibody complex, thereby identifying an antibody to dengue virus serotype (1, 2, 3, and/or 4) in the biological sample from the subject.

A further aspect of the invention provides a method of identifying an antibody to a specific dengue virus serotype or combinations thereof in a biological sample from a subject, comprising: a) contacting a biological sample from a subject that has been administered an immunogenic composition comprising a particular E glycoprotein of this invention with the E glycoprotein under conditions whereby an antigen/antibody complex can form; and c) detecting formation of an antigen/antibody complex, thereby identifying an antibody dengue virus serotype (1, 2, 3, and/or 4) in the biological sample from the subject.

The present invention additionally provides a method of identifying an immunogenic composition that induces an antibody to a specific dengue virus serotype or combination thereof in a subject, the method comprising: a) contacting a biological sample from a subject that has been administered an immunogenic composition comprising a particular E glycoprotein of this invention with the E glycoprotein under conditions whereby an antigen/antibody complex can form; and b) detecting formation of an antigen/antibody complex, thereby identifying an immunogenic composition that induces an antibody to the specific dengue virus serotype or combination thereof in the subject.

A further embodiment of the invention is a method of identifying an immunogenic composition that induces a neutralizing antibody to a specific dengue virus serotype or combination thereof in a subject, comprising: a) administering an immunogenic composition comprising a particular E glycoprotein to a subject in an amount effective to induce an antibody response to the E glycoprotein; b) contacting a biological sample from the subject with the E glycoprotein of (a) under conditions whereby an antigen/antibody complex can form; and c detecting formation an antigen/antibody complex, thereby identifying an immunogenic composition that induces a neutralizing antibody to the specific dengue virus serotype or combination thereof in the subject.

### BRIEF DESCRIPTION OF THE DRAWINGS

FIG. 1 shows DENV1/3EDI, EDII and EDIII chimeras depicted in a hexameric raft formation with 6 antiparallel E glycoprotein monomers forming 3 parallel dimers. The prM is pictured to the right of the EDII and EDIII chimeras (FIG. 1 panel A). DENV3 E-protein 3-fold, 2-fold and 5-fold symmetry are represented on the whole virion with DENV1/3 EDI, DENV1/3 EDII and DENV1/3 EDIII respectively (FIG. 1 panel B).

FIG. 2 shows Vero81 NHP cells infected in triplicate and virus titers measured by foci stain at 48 hours per infection, circles=triplicate titers (FIG. 2 panel A). Confirmation of DENV1 and 3 epitopes in the DENV1/3 EDI, II and III panel plus parental strains using DENV1 and 3 mAb arrays (FIG. 2 panel B). E and prM proteins were visualized through Western Blot (FIG. 2 panel C) and prM/E ratios determined, with lower numbers indicating greater maturity (FIG. 2 panel D).

FIG. 3 shows DENV3/3 EDII, G-III into G-IV parent (9 residues) and 4 DENV3/3, G-III into G-IV cluster chimeras (C₁-C₄) encoding between 2-6 DENV3 G-III residues. C2 and C3 together make the residues encoded into C4 (FIG. 3 panel A). DENV1/3 EDII-targeting mAbs 115 and 419 neutralize C3 and C4, with the critical residues lying in C3 (FIG. 3 panel B). The critical mAb-115 and 419 epitope residues are depicted on the whole virion, and are located at the EDII-EDII dimer interface (E62, G63, Q120, P124) (FIG. 3 panel C). Novel EDII-targeting mAbs-P3D05 and -P7C07 were tested via FRNT for neutralization with a panel of DENV3/3 envelope genotype I-IV chimeras and exhibited similar potency between strains (FIG. 3 panel D).

FIG. 4 shows sera depleted with antigens adsorbed onto magnetic beads using 3 conditions: BSA control, DENV1, 2, 4 virions or DENV3 virions (FIG. 4 panel A). As a proof of concept, DENV3 EDI, II or III targeting antibodies were spiked into NHS, depleted and tested against the DENV1/3 chimeric panel. The BSA and DENV1, 2, 4 depletions did not lose any neutralization of the chimeric panel, but the DENV3 depleted sera lost all neutralization with the panel (FIG. 4 panel B).

FIG. 5 shows 6 DENV3 primary natural serum, 3 from the Adult Arbovirus Travelers Cohort (ATC A-C) and 3 from the Nicaraguan Pediatric Dengue Cohort Study (NPDCS A-C) were depleted with BSA, DENV1, 2, 4 or DENV3 virus on a magnetic bead and the remaining sera tested with a wildtype panel (DENV1, 2, 4 and DENV3, GII and DENV3, GIII) (FIG. 5 panel A) or the DENV1/3 EDI-III chimeric panel and wildtype DENV1 and DENV3, GIII (FIG. 5 panel B). The BSA depleted denotes the control where total antibodies are present. The DENV1, 2, 4 column denotes DENV3 TS antibodies and the DENV3 column denotes any background neutralization, which is then subtracted from the BSA and DENV1,2,4 columns.

FIG. 6 shows 6 NIH DENV3 monovalent vaccine serum samples (NIH A-F) and a monoclonal control (EDEC10) tested undepleted in an FRNT with DENV1, DENV3, GIII, and the DENV1/3 EDI, EDII and EDIII chimeric panel.

FIG. 7 shows data plots of neutralizing antibody titers from DENV monovalent vaccine depleted sera to measure DENV3 serotype-specific (TS) responses. When tested for neutralization of DENV3 G-II and G-III, DENV3 G-II (geometric mean fold change=10) was more efficiently neutralized by DENV3 TS vaccine serum than DENV3 G-II in all cases. Data plots shown over two panels, FIG. 7 panels A and B.

FIG. 8 shows a data plot of antigenic cartography, plotting differences in the neutralizing polyclonal response between viruses in 2-dimensional space.

FIG. 9 shows designed DV4/2 chimeric viruses with residues of DV2 placed into a DV4 backbone. FIG. 9 panels A and B show growth curves of icDV4/2 ED chimeric viruses and parent viruses at MOI 0.1 on Vero81 cells comparing original recovery and reconstructed icDV4/2 EDI+TC and icDV4/2 EDII+TC. FIG. 9 panel C shows (left) representative Western blot staining for prM and E of viral stocks harvested after 144 hr infection on Vero81 and (right) quantified band densities. FIG. 9 panel D shows EC40 of described monoclonal antibodies against icDV4/2 ED chimeric viruses and parents on Vero81 cells in live virus neutralization assays.

### DETAILED DESCRIPTION OF THE INVENTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise. For example, “a” cell can mean one cell or a plurality of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, +1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the term “nucleic acid” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

The terms “nucleic acid segment,” “nucleotide sequence,” “nucleic acid molecule,” or more generally “segment” will be understood by those in the art as a functional term that includes both genomic DNA sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art. Thus, all or a portion of the nucleic acids of the present codons may be synthesized using codons preferred by a selected host. Species-preferred codons may be determined, for example, from the codons used most frequently in the proteins expressed in a particular host species. Other modifications of the nucleotide sequences may result in mutants having slightly altered activity.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol. 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, the term “polypeptide” encompasses both peptides and proteins (including fusion proteins), unless indicated otherwise.

As used herein, the term “chimera,” “chimeric,” and/or “fusion protein” refer to an amino acid sequence (e.g., polypeptide) generated non-naturally by deliberate human design comprising, among other components, an amino acid sequence of a protein of interest and/or a modified variant and/or active fragment thereof (a “backbone”), wherein the protein of interest comprises modifications (e.g., substitutions such as singular residues and/or contiguous regions of amino acid residues) from different wild type reference sequences (chimera), optionally linked to other amino acid segments (fusion protein). The different components of the designed protein may provide differing and/or combinatorial function. Structural and functional components of the designed protein may be incorporated from differing and/or a plurality of source material. The designed protein may be delivered exogenously to a subject, wherein it would be exogenous in comparison to a corresponding endogenous protein.

As used herein with respect to nucleic acids, the term “operably linked” refers to a functional linkage between two or more nucleic acids. For example, a promoter sequence may be described as being “operably linked” to a heterologous nucleic acid sequence because the promoter sequence initiates and/or mediates transcription of the heterologous nucleic acid sequence. In some embodiments, the operably linked nucleic acid sequences are contiguous and/or are in the same reading frame.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein, an “isolated” polynucleotide (e.g., an “isolated nucleic acid” or an “isolated nucleotide sequence”) means a polynucleotide at least partially separated from at least some of the other components of the naturally occurring organism or virus, for example, the cell or viral structural components or other polypeptides or nucleic acids commonly found associated with the polynucleotide. Optionally, but not necessarily, the “isolated” polynucleotide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polynucleotide is at least about 1%, 5%, 10%, 20%, 30%, 40%, 50%, 60%, 70%, 80%, 90%, 95% or more pure.

An “isolated” polypeptide means a polypeptide that is at least partially separated from at least some of the other components of the naturally occurring organism or virus, for example, the cell or viral structural components or other polypeptides or nucleic acids commonly found associated with the polypeptide. Optionally, but not necessarily, the “isolated” polypeptide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polypeptide is at least about 1%, 5%, 10%, 20%, 30%, 40%, 50%, 60%, 70%, 80%, 90%, 95% or more pure.

Furthermore, an “isolated” cell is a cell that has been partially or completely separated from other components with which it is normally associated in nature. For example, an isolated cell can be a cell in culture medium and/or a cell in a pharmaceutically acceptable carrier.

The term “endogenous” refers to a component naturally found in an environment, i.e., a gene, nucleic acid, miRNA, protein, cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5,6,7, 8,9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6,7, 8,9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

The terms “immunogen” and “antigen” are used interchangeably herein and mean any compound (including polypeptides) to which a cellular and/or humoral immune response can be directed. In particular embodiments, an immunogen or antigen can induce a protective immune response against the effects of dengue virus infection. A molecule capable of antibody and/or immune response stimulation may be referred to as antigenic/immunogenic, and can be said to have the ability of antigenicity/immunogenicity. The binding site for an antibody within an antigen and/or immunogen may be referred to as an epitope (e.g., an antigenic epitope). The term “vaccine antigen” as used herein refers to such an antigen/immunogen as used as a vaccine, e.g., a prophylactic, preventative, and/or therapeutic vaccine.

“Effective amount” as used herein refers to an amount of a vector, nucleic acid, epitope, polypeptide, cell, particle, VLP, composition or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

The term “immunogenic amount” or “effective immunizing dose,” as used herein, unless otherwise indicated, means an amount or dose sufficient to induce an immune response (which can optionally be a protective response) in the treated subject that is greater than the inherent immunity of non-immunized subjects. An immunogenic amount or effective immunizing dose in any particular context can be routinely determined using methods known in the art.

The terms “vaccine,” “vaccination” and “immunization” are well-understood in the art, and are used interchangeably herein. For example, the terms vaccine, vaccination or immunization can be understood to be a process or composition that increases a subject's immune reaction to an immunogen (e.g., by providing an active immune response), and therefore its ability to resist, overcome and/or recover from infection (i.e., a protective immune response).

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the terms “prime boost immunization,” “prime boost administration,” or “prime and booster” refer to an administration (e.g., immunization) regimen that comprises administering to a subject a primary/initial (priming) administration (e.g., of one or more chimeric coronavirus S protein of the present invention) and at least one secondary (boosting) administration. In some embodiments, the priming administration and the at least one boosting administration may comprise the same composition, administered in multiple (one or more) repetitions. In some embodiments, the priming administration and the at least one boosting administration may comprise different types of compositions, such as different types of chimeric coronavirus S proteins of the present invention.

As used herein, the terms “prime immunization,” “priming immunization,” “primary immunization” or “prime” refer to primary antigen stimulation by using a chimeric coronavirus S protein according to the instant invention.

The term “boost immunization,” “boosting immunization,” “secondary immunization”, or “boost” refers to additional administration (e.g., immunization) of a chimeric coronavirus S protein of the present invention administered to a subject after a primary administration. In some embodiments, the boost immunization may be administered at a dose higher than, lower than, and/or equal to the dose administered as a primary immunization, e.g., when the boost immunization is administered alone without priming.

The prime and boost vaccine compositions may be administered via the same route or they may be administered via different routes. The boost vaccine composition may be administered one or several times at the same or different dosages. It is within the ability of one of ordinary skill in the art to optimize prime-boost combinations, including optimization of the timing and dose of vaccine administration.

As used herein, by “isolate” or “purify” (or grammatical equivalents) a vector, it is meant that the vector is at least partially separated from at least some of the other components in the starting material.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold, and/or at least about 5%, 10%, 15%, 20%, 25%, 30%, 35%, 40%, 45%, 50%, 55%, 60%, 65%, 70%, 75%, 80%, 85%, 90%, 95%, or 100% or more, or any value or range therein.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts may be referred to as “transcription products” and encoded polypeptides may be referred to as “translation products.” Transcripts and encoded polypeptides may be collectively referred to as “gene products.” If the polynucleotide is derived from genomic DNA, expression may include splicing of the mRNA in a eukaryotic cell. The expression product itself, e.g., the resulting nucleic acid or protein, may also be said to be “expressed.” An expression product can be characterized as intracellular, extracellular, or secreted. The term “intracellular” means something that is inside a cell. The term “extracellular” means something that is outside a cell. A substance is “secreted” by a cell if it appears in significant measure outside the cell, from somewhere on or inside the cell.

By the terms “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder. In representative embodiments, the terms “treat,” “treating” or “treatment of” (and grammatical variations thereof) refer to a reduction in the severity of viremia and/or a delay in the progression of viremia, with or without other signs of clinical disease.

A “treatment effective” amount as used herein is an amount that is sufficient to treat (as defined herein) the subject. Those skilled in the art will appreciate that the therapeutic effects need not be complete or curative, as long as some benefit is provided to the subject.

The term “prevent,” “preventing” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the terms “prevent,” “preventing” or “prevention of” (and grammatical variations thereof) refer to prevention and/or delay of the onset and/or progression of viremia in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The efficacy of treating and/or preventing dengue virus infection by the methods of the present invention can be determined by detecting a clinical improvement as indicated by a change in the subject's symptoms and/or clinical parameters (e.g., viremia), as would be well known to one of skill in the art.

Unless indicated otherwise, the terms “protect,” “protecting,” “protection” and “protective” (and grammatical variations thereof) encompass both methods of preventing and treating dengue virus infection in a subject, whether against one or multiple strains, genotypes or serotypes of dengue virus.

The terms “protective” immune response or “protective” immunity as used herein indicates that the immune response confers some benefit to the subject in that it prevents or reduces the incidence and/or severity and/or duration of disease or any other manifestation of infection. For example, in representative embodiments, a protective immune response or protective immunity results in reduced viremia, whether or not accompanied by clinical disease. Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “subject” of the invention includes any animal susceptible to dengue virus infection. Such a subject is generally a mammalian subject (e.g., a laboratory animal such as a rat, mouse, guinea pig, rabbit, primates, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. Subjects of the invention can be a subject known or believed to be at risk of infection by dengue virus. Alternatively, a subject according to the invention can also include a subject not previously known or suspected to be infected by dengue virus or in need of treatment for dengue virus infection.

Subjects may be treated for any purpose, such as for eliciting a protective immune response or for eliciting the production of antibodies in that subject, which antibodies can be collected and used for other purposes such as research or diagnostic purposes or for administering to other subjects to produce passive immunity therein, etc.

Subjects include males and/or females of any age, including neonates, juvenile, mature and geriatric subjects. With respect to human subjects, in representative embodiments, the subject can be an infant (e.g., less than about 12 months, 10 months, 9 months, 8 months, 7 months, 6 months, or younger), a toddler (e.g., at least about 12, 18 or 24 months and/or less than about 36, 30 or 24 months), or a child (e.g., at least about 1, 2, 3, 4 or 5 years of age and/or less than about 14, 12, 10, 8, 7, 6, 5, or 4 years of age). In embodiments of the invention, the subject is a human subject that is from about 0 to 3, 4, 5, 6, 9, 12, 15, 18, 24, 30, 36, 48 or 60 months of age, from about 3 to 6, 9, 12, 15, 18, 24, 30, 36, 48 or 60 months of age, from about 6 to 9, 12, 15, 18, 24, 30, 36, 48 or 60 months of age, from about 9 to 12, 15, 18, 24, 30, 36, 48 or 60 months of age, from about 12 to 18, 24, 36, 48 or 60 months of age, from about 18 to 24, 30, 36, 48 or 60 months of age, or from about 24 to 30, 36, 48 or 60 months of age.

In embodiments of the invention, the subject has maternal antibodies to dengue virus.

A “subject in need” of the methods of the invention can be a subject known to be, or suspected of being, infected with, or at risk of being infected with, dengue virus.

The term “administering” or “administration” of a composition of the present invention to a subject includes any route of introducing or delivering to a subject a compound to perform its intended function (e.g., for use as a vaccine antigen). Administration includes self-administration and the administration by another.

A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art.

### Compositions

The present invention is based in part on the unexpected discovery that epitope regions that define a DENV serotype can be transferred into a protein backbone of a different DENV serotype to create a chimeric molecule that contains antibody targets for both serotypes, thereby functioning as a bivalent vaccine that can induce neutralizing antibodies against two different DENV serotypes from a single source. Thus, in one embodiment, the present invention provides a platform for construction of a chimeric dengue virus E glycoprotein backbone that comprises amino acid substitutions that introduce epitopes that are recognized by an antibody that is reactive with a dengue virus serotype that is different from the dengue virus serotype of the dengue virus E glycoprotein backbone.

Accordingly, the present invention provides a chimeric dengue virus E glycoprotein comprising a dengue virus E glycoprotein backbone from a first dengue virus serotype (e.g., DENV 1, 2, 3, or 4), and one or more regions (e.g., domains) of amino acid substitutions from one or more other dengue virus serotypes that is different from the dengue virus E glycoprotein backbone. This invention additionally relates to the use of the chimeras of the present invention in various methods, such as to produce an immune response, treat a coronavirus infection, prevent a disease or disorder associated with a dengue virus infection and/or caused by a dengue virus infection, protect a subject from the effects of a dengue virus infection, among others. The present invention also provides chimeric dengue virus prM glycoproteins, as well as nucleic acid molecules, vectors, particles, populations, and compositions comprising the same, and methods of using the chimeric dengue virus E glycoproteins and/or chimeric dengue virus prM glycoproteins of this invention.

In some embodiments, a dengue virus E glycoprotein backbone of the present invention may be from dengue virus serotype 1, dengue virus serotype 2, dengue virus serotype 3, or dengue virus serotype 4.

In some embodiments, an antibody that is reactive with a dengue virus serotype that is different from the dengue virus serotype of the dengue virus E glycoprotein backbone may be an antibody that is reactive with dengue virus serotype 3. In other embodiments, the antibody that is reactive with a dengue virus serotype that is different from the dengue virus serotype of the dengue virus E glycoprotein backbone is an antibody that is reactive with dengue virus serotype 1, dengue virus serotype 2 or dengue virus serotype 4.

It would be understood that any combination of a first dengue virus serotype for the dengue virus E glycoprotein backbone and a second dengue virus serotype that is the target of the antibody that recognizes the epitope introduced into the E glycoprotein backbone can be used, provided that the first dengue virus serotype and the second dengue virus serotype are different (i.e., not the same serotype).

In some embodiments, a chimeric dengue virus E glycoprotein of the present invention may comprise one or more amino acid substitutions, deletions, and/or insertions as listed in Table 3 and/or shown in the alignment of Table 1, wherein the numbering is based on the reference amino acid sequence of an E glycoprotein of dengue virus serotype 1 (DENV1) identified as SEQ ID NO:11.

In some embodiments, a chimeric dengue virus E glycoprotein of the present invention may comprise one or more amino acid substitutions, deletions, and/or insertions as listed in Table 4 and/or shown in the alignment of Table 2, wherein the numbering is based on the reference amino acid sequence of an E glycoprotein of dengue virus serotype 4 (DENV4) identified as SEQ ID NO:14.

A DENV1 backbone may comprise the amino acid sequence of any DENV1 genotype and/or strain and/or isolate currently known or as yet identified and/or isolated. Non-limiting examples of DENV1 genotypes, strains, and/or isolates include Genotypes I, II, III, IV, and V, strains Western Pacific 1974, and GenBank® Accession No. U88535.1.

A DENV3 backbone may comprise the amino acid sequence of any DENV3 genotype and/or strain and/or isolate currently known or as yet identified and/or isolated. Non-limiting examples of DENV3 genotypes, strains, and/or isolates include Genotype I, II, III, IV, strains such as Sri Lanka 1989, Indonesia 1982, Thailand 1995, Cuba 2002, and Puerto Rico 1977, and GenBank® Accession Nos. JQ411814.1 (“UNC3001”), DQ401690.1, AY676376, AY02031, and AY146761.

A DENV2 backbone may comprise the amino acid sequence of any DENV2 genotype and/or strain and/or isolate currently known or as yet identified and/or isolated. Non-limiting examples of DENV2 genotypes, strains, and/or isolates include Genotypes Cosmopolitan, Asian-American, Asian I, and Asian II, strains such as S-16803 (GenBank® Accession No. GU289914.1), India 2001 (GenBank® Accession No. DQ448237b), Puerto Rico 2003 (GenBank® Accession No. EU687235b), USA 2009 (GenBank® Accession No. HQ541798b), Indonesia 2016 (GenBank® Accession No. NM173162), Thailand 2016 (GenBank® Accession No. LC410185b), and GenBank® Accession No. NC_001474.2.

A DENV4 backbone may comprise the amino acid sequence of any DENV4 genotype and/or strain and/or isolate currently known or as yet identified and/or isolated. Non-limiting examples of DENV4 genotypes, strains, and/or isolates include Genotypes I, IIa, IIb, III, IV, and V and strains such as GenBank® Accession Nos. KF543272, JN832541, FJ882599, KJ160504.1, AY618940, AF231724, and JF262783.

In some embodiments, an amino acid residue is substituted adjacent to an insertion. In some embodiments, a substitution may comprise a deletion of one or more amino acid residues. The substitution(s) and residue positions may be described in one or more ways that are redundant in generating the same resultant amino acid sequence. Thus, a disclosure of one such description is herein considered a disclosure of each and inclusive of all such redundant disclosures. Thus, wherein the resultant amino acid sequence is identical, any disclosure provided herein describing one way to produce the resultant amino acid sequence is considered a disclosure of each and inclusive of all such redundant disclosures.

In some embodiments, the chimeric dengue virus E glycoprotein of this invention can comprise, consist essentially of or consist of the amino acid sequence of SEQ ID NO:1, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 1. DENV 1/3 EDII “small” 438-2 E
chimera
MRCVGIGNRDFVEGLSGATWVDVVLEHGSCVTTMAKDKPTLDIELLKTEV
TNLATLRKLCIEGKITNITTDSRCPTQGEAVLPEEQDQNYVCKHTYVDRG
WGNGCGLFGKGSLVTCAKFQCLEPIEGKVVQYENLKYSVIVTVHTGDQHQ
VGNETTEHGTIATITPQAPTSEIQLTDYGALTLDCSPRTGLDFNEMILLT
MKNKAWMVHRQWFFDLPLPWTSGATTETPTWNRKELLVTFKNAHAKKQEV
VVLGSQEGAMHTALTGATEIQNSGTTTIFAGHLKCRLKMDKLTLKGMSYV
MCTGSFKLEKEVAETQHGTVLVQIKYEGTDAPCKIPFSTQDEKGVTQNGR
LITANPIVTDKEKPVNIEAEPPFGESYIVIGAGEKALKLSWFKKGSSIGK
MFEATARGARRMAILGDTAWDFGSIGGVFTSVGKLVHQIFGTAYGVLFSG
VSWTMKIGIGVLLTWLGLNSRSTSLSMTCIAVGLVTLYLGVMVQA

In some embodiments, the chimeric dengue virus E glycoprotein of this invention can comprise, consist essentially of or consist of the amino acid sequence of SEQ ID NO:2, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 2. DENV 1/3 EDII “large” 438-3 E
chimera
MRCVGIGNRDFVEGLSGATWVDVVLEHGSCVTTMAKDKPTLDIELLKTEA
TQLATLRKLCIEGKITNITTDSRCPTQGEAVLPEEQDQNYVCKHTYVDRG
WGNGCGLFGKGSLVTCAKFQCLEPIEGKVVQYENLKYSVIVTVHTGDQHQ
VGNETTEHGTIATITPQAPTSEIQLTDYGALTLDCSPRTGLDFNEMILLT
MKNKAWMVHRQWFFDLPLPWTSGATTETPTWNRKELLVTFKNAHAKKQEV
VVLGSQEGAMHTALTGATEIQNSGGTSIFAGHLKCRLKMDKLTLKGMSYV
MCTGSFKLEKEVAETQHGTVLVQIKYEGTDAPCKIPFSTQDEKGVTQNGR
LITANPIVTDKEKPVNIEAEPPFGESYIVIGAGEKALKLSWFKKGSSIGK
MFEATARGARRMAILGDTAWDFGSIGGVFTSVGKLVHQIFGTAYGVLFSG
VSWTMKIGIGVLLTWLGLNSRSTSLSMTCIAVGLVTLYLGVMVQA

In some embodiments, the present invention provides a chimeric E glycoprotein that may interact with a chimeric prM protein. Non-limiting examples of a chimeric E glycoprotein that may interact with a chimeric prM protein include chimeric E glycoproteins with one or more substitutions in domain II (DII).

In some embodiments, the present invention provides a chimeric E glycoprotein that may interact with a chimeric prM protein (e.g., a chimeric DENV1 prM protein) comprising, consisting essentially of or consisting of the amino acid sequence of SEQ ID NO:7, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 7. DENV 1/3 EDII “small” 438-2 and/or
EDII “large” 438-3 prM chimera
FHLTSRDGEPRMIVGKNERGKSLLFKTASGINMCTLIAMDLGEMCDDTLT
YKCPHITEVEPEDIDCWCNLTSTWVTYGTCNQAGEHRRDKRSVALAPHVG
MGLDTRTQTWMSSEGAWKQIQKVETWALRHPGFTVIALFLAHAIGTSITQ
KGIIFILLMLVTPSMA

In some embodiments, the chimeric dengue virus E glycoprotein of this invention can comprise, consist essentially of or consist of the amino acid sequence of SEQ ID NO:3, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 3. DENV 1/3 EDIII E chimera
MRCVGIGNRDFVEGLSGATWVDVVLEHGSCVTTMAKDKPTLDIELLKTEV
TNPAVLRKLCIEAKISNTTTDSRCPTQGEATLVEEQDTNFVCRRTFVDRG
WGNGCGLFGKGSLITCAKFKCVTKLEGKIVQYENLKYSVIVTVHTGDQHQ
VGNETTEHGTIATITPQAPTSEIQLTDYGALTLDCSPRTGLDFNEMVLLT
MKKKSWLVHKQWFLDLPLPWTSGASTSQETWNRQDLLVTFKTAHAKKQEV
VVLGSQEGAMHTALTGATEIQTSGTTTIFAGHLKCRLKMDKLTLKGMSYA
MCTNTFVLKKEVSETQHGTILIKVEYKGEDAPCKIPFSTEDGQGKAHNGR
LITANPVVTKKEEPVNIEAEPPFGESNIVIGIGDNQHTGGRFKKGSSIGK
MFEATARGARRMAILGDTAWDFGSVGGVLNSLGKMVHQIFGSAYTALFSG
VSWVMKIGIGVLLTWIGLNSKNTSMSFSCIAIGIITLYLGPYFAT

In some embodiments, the present invention provides a chimeric E glycoprotein that may interact with a wildtype prM protein, e.g., a wildtype DENV1 prM protein. Non-limiting examples of a chimeric E glycoprotein that may interact with a wildtype prM protein include chimeric E glycoproteins with one or more substitutions in domain I (DI) and/or domain III (DIII).

In some embodiments, the present invention provides a chimeric E glycoprotein that may interact with a prM protein comprising, consisting essentially of or consisting of the amino acid sequence of SEQ ID NO:8, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 8. DENV1 wildtype prM
FHLTTRGGEPHMIVSKQERGKSLLFKTSAGVNMCTLIAMDLGELCEDTMT
YKCPRITETEPDDVDCWCNATETWVTYGTCSQTGEHRRDKRSVALAPHVG
LGLETRTETWMSSEGAWKQIQKVETWALRHPGFTVIALFLAHAIGTSITQ
KGIIFILLMLVTPSMA

In some embodiments, the chimeric dengue virus E glycoprotein of this invention can comprise, consist essentially of or consist of the amino acid sequence of SEQ ID NO:4, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 4. DENV 4/2 EDI E chimera
MRCIGISNRDFVEGVSGGSWVDIVLEHGSCVTTMAQNKPTLDFELIKTEA
KEVALLRTYCIEASISNITTATRCPTQGEPYLKEEQDQQYICRRDVVDRG
WGNGCGLFGKGGVVTCAKFSCSGKITGNLVQPENLEYTIVVTPHSGEEHA
VGNDTGKHGKEIKVTPQSSITEAELTGYGTVTMECSPRTGLDFNEMILMK
MKKKTWLVHKQWFLDLPLPWTAGADTSEVHWNYKERMVTFKVPHAKRQDV
TVLGSQEGAMHSALAGATEVDSGDGNHLFAGHLKCRLRMDKLQLKGMSYT
MCSGKFKIDKEMAETQHGTTVVKVKYEGAGAPCKVPIEIRDVNKEKVVGR
VISSTPLAENTNSVTNIELEPPFGDSYIVIGVGNSALTLHWFRKGSSIGK
MFESTYRGAKRMAILGETAWDFGSVGGLFTSLGKAVHQVFGSVYTTMFGG
VSWMIRILIGFLVLWIGTNSRNTSMAMTCIAVGGITLFLGFTVQA

In some embodiments, the chimeric dengue virus E glycoprotein of this invention can comprise, consist essentially of or consist of the amino acid sequence of SEQ ID NO:6, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 6. DENV 4/2 EDIII E chimera
MRCVGVGNRDFVEGVSGGAWVDLVLEHGGCVTTMAQGKPTLDFELTKTTA
KEVALLRTYCIEASISNITTATRCPTQGEPYLKEEQDQQYICRRDVVDRG
WGNGCGLFGKGGVVTCAKFSCSGKITGNLVQIENLEYTVVVTVHNGDTHA
VGNDTSNHGVTATITPRSPSVEVKLPDYGELTLDCEPRSGIDFNEMILMK
MKKKTWLVHKQWFLDLPLPWTAGADTSEVHWNYKERMVTFKVPHAKRQDV
TVLGSQEGAMHSALAGATEVDSGDGNHMFAGHLKCKVRMEKLRIKGMSYS
MCTGKFKVVKEIAETQHGTIVIRVQYEGDGSPCKIPFEIMDLEKRHVLGR
LITVNPIVTEKDSPVNIEAEPPFGDSYIIIGVDPGQLKLNWFKKGSSIGK
MFESTYRGAKRMAILGETAWDFGSVGGLFTSLGKAVHQVFGSVYTTMFGG
VSWMIRILIGFLVLWIGTNSRNTSMAMTCIAVGGITLFLGFTVQA

In some embodiments, the present invention provides a chimeric E glycoprotein that may interact with a wildtype prM protein, e.g., a wildtype DENV4 prM protein. Non-limiting examples of a chimeric E glycoprotein that may interact with a wildtype prM protein include chimeric E glycoproteins with one or more substitutions in domain I (DI) and/or domain III (DIII).

In some embodiments, the present invention provides a chimeric E glycoprotein that may interact with a prM protein comprising, consisting essentially of or consisting of the amino acid sequence of SEQ ID NO:9, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 9. DENV4 wildtype prM
FHLSTRDGEPLMIVAKHERGRPLLFKTTEGINKCTLIAMDLGEMCEDTVT
YKCPLLVNTEPEDIDCWCNLTSTWVMYGTCTQSGERRREKRSVALTPHSG
MGLETRAETWMSSEGAWKHAQRVESWILRNPGFALLAGFMAYMIGQTGIQ
RTVFFVLMMLVAPSYG

In some embodiments, the chimeric dengue virus E glycoprotein of this invention can comprise, consist essentially of or consist of the amino acid sequence of SEQ ID NO:5, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 5. DENV 4/2 EDII E chimera
MRCVGVGNRDFVEGVSGGAWVDLVLEHGGCVTTMAQGKPTLDFELIKTEA
KQPATLRKYCIEAKLTNTTTESRCPTQGEPSLNEEQDKRFVCKHSMVDRG
WGNGCGLFGKGGIVTCAMFTCKKNMEGKVVQPENLEYTVVVTVHNGDTHA
VGNDTSNHGVTATITPRSPSVEVKLPGYGELTLDCEPRSGLDFNEMVLLQ
MENKAWLVHRQWFLDLPLPWLPGADTQGSNWIQKETLVTFKNPHAKKQDV
VVLGSQEGAMHTALTGATEIQMSSGNLLFTGHLKCKVRMEKLRIKGMSYT
MCSGKFSIDKEMAETQHGTTVVKVKYEGAGAPCKVPIEIRDVNKEKVVGR
VISSTPLAENTNSVTNIELEPPFGDSYIVIGVGNSALTLHWFRKGSSIGK
MFESTYRGAKRMAILGETAWDFGSVGGLFTSLGKAVHQVFGSVYTTMFGG
VSWMIRILIGFLVLWIGTNSRNTSMAMTCIAVGGITLFLGFTVQA

In some embodiments, the present invention provides a chimeric E glycoprotein that may interact with a chimeric prM protein (e.g., a chimeric DENV4 prM protein) comprising, consisting essentially of or consisting of the amino acid sequence of SEQ ID NO:10, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 10. DENV4/2 EDII prM chimera
FHLTTRNGEPHMIVSRQEKGKSLLFKTEDGVNMCTLMAMDLGELCEDTIT
YNCPLLRQNEPEDIDCWCNSTSTWVTYGTCTTTGEHRRGKRSVALVPHVG
MGLETRTETWMSSEGAWKHAQRVESWILRNPGFALLAGFMAYMIGQTGIQ
RTVFFVLMMLVAPSYG

The amino acid residue positions of the substitutions that can be made to produce the desired chimeric E and/or prM glycoproteins of the present invention can be readily determined by one of ordinary skill in the art according to the teachings herein and according to protocols well known in the art. The amino acid residue numbering provided in the amino acid sequences set forth here is based on the reference sequence of DENV1 or DENV4 wild type E glycoprotein, as provided herein (SEQ ID NO:11 and 14). However it would be readily understood by one of ordinary skill in the art that the equivalent amino acid positions in other dengue virus E glycoproteins can be readily identified and employed in the production of the chimeric E glycoproteins of this invention.

It would be understood that the modifications described above provide multiple examples of how the amino acid sequences described herein can be obtained and that, due to the degeneracy of the amino acid codons, numerous other modifications can be made to a nucleotide sequence encoding an E glycoprotein or fragment thereof to obtain the desired amino acid sequence. The present invention provides additional non limiting examples of nucleic acids and/or polypeptides of this invention that can be used in the compositions and methods described herein in the SEQUENCES section provided herein.

In some embodiments, the present invention provides a chimeric flavivirus E glycoprotein in which amino acid substitutions are made to introduce a dengue virus epitope into a flavivirus E glycoprotein from a flavivirus that is not a dengue virus. Thus, in some embodiments, the present invention provides a flavivirus E glycoprotein comprising a chimeric E glycoprotein comprising a flavivirus E glycoprotein backbone that is not a dengue virus E glycoprotein backbone, wherein the flavivirus E glycoprotein backbone comprises amino acid substitutes that introduce an epitope that is recognized by an antibody that is reactive with a dengue virus.

Nonlimiting examples of flaviviruses that can be used include yellow fever virus (YFV) (e.g., GenBank© Accession No. JX503529) Japanese encephalitis virus (JEV) (e.g., GenBank© Accession No. U14163), West Nile virus (WNV) (e.g., GenBank© Accession No. DQ211652) and any other flavivirus now known or later identified.

The present invention further provides an isolated nucleic acid molecule encoding the chimeric dengue virus E glycoprotein and/or the chimeric or wildtype dengue virus prM glycoprotein of this invention. In some embodiments, a nucleic acid molecule of this invention may be a cDNA molecule. In some embodiments, a nucleic acid molecule of this invention may be an mRNA molecule.

Also provided is a vector, plasmid or other nucleic acid construct comprising the isolated nucleic acid molecule of this invention.

A vector can be any suitable means for delivering a polynucleotide to a cell. A vector of this invention can be an expression vector that contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise viral nucleic acid including, but not limited to, poxvirus, vaccinia virus, adenovirus, retrovirus, alphavirus and/or adeno-associated virus nucleic acid. The nucleic acid molecule or vector of this invention can also be in a liposome or a delivery vehicle, which can be taken up by a cell via receptor-mediated or other type of endocytosis. The nucleic acid molecule of this invention can be in a cell, which can be a cell expressing the nucleic acid whereby a chimeric E glycoprotein of this invention is produced in the cell (e.g., a host cell). In addition, the vector of this invention can be in a cell, which can be a cell expressing the nucleic acid of the vector whereby a chimeric E glycoprotein of this invention is produced in the cell. It is also contemplated that the nucleic acid molecules and/or vectors of this invention can be present in a host organism (e.g., a transgenic organism), which expresses the nucleic acids of this invention and produces a chimeric E glycoprotein of this invention. In some embodiments, the vector is a plasmid, a viral vector, a bacterial vector, an expression cassette, a transformed cell, or a nanoparticle. For example, in some embodiments a chimeric E glycoprotein of the present invention may be used in combination (e.g., in scaffold(s) and/or conjugated with) other molecules such as, but not limited to, nanoparticles, e.g., as delivery devices.

Types of nanoparticles of this invention for use as a vector and/or delivery device include, but are not limited to, polymer nanoparticles such as PLGA-based, PLA-based, polysaccharide-based (dextran, cyclodextrin, chitosan, heparin), dendrimer, hydrogel; lipid-based nanoparticles such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales. Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticles such as lipid microparticles, micelles; inorganics-based microparticles such as superparamagnetic iron oxide microparticles, platin microparticles and the like as are known in the art. These particles may be generated and/or have materials be absorbed, encapsulated, or chemically bound through known mechanisms in the art.

In some embodiments, a nanoparticle vector of the present invention may be an mRNA lipid nanoparticle (mRNA-LNP), a nucleic acid vaccine (NAV), or other nucleic acid lipid nanoparticle compositions, such as described in U.S. Pat. Nos. 9,868,692; 9,950,065; 10,041,091; 10,576,146; 10,702,600; WO2015/164674; US2019/0351048; US2020/297634; WO2020/097548; and Buschmann et al. 2021 Vaccines 9(65) doi.org/10.3390/vaccines9010065; Laczk6 et al. 2020 Immunity 53:724-732; and Pardi et al. 2018 Nat. Rev. Drug Discov. 17:261-279, the disclosures of each of which are incorporated herein by reference in their entireties.

It is known in the art that many attempts to produce dengue virus vaccines result in the production of non-neutralizing antibodies, which may increase the likelihood of pathology upon subsequence exposure to natural infection or vaccine. Another approach to provide an engineered epitope is to deliver all or a portion of the dengue virus E protein incorporated into another flavivirus particle or VLP. In representative embodiments, the heterologous flavivirus is West Nile virus or Yellow Fever virus. Portions of the E protein can be grafted into the E protein of the heterologous flavivirus backbone, e.g., to reduce the generation of non-neutralizing dengue virus antibodies to non-neutralizing epitopes present in the dengue virus E protein and/or other dengue virus structural proteins.

Thus, a chimeric flavivirus or chimeric flavivirus VLP can present the quaternary dengue virus epitope in proper conformation while reducing the generation of non-neutralizing antibodies to other portions of the dengue virus E protein and/or other structural proteins that are not presented in the chimeric flavivirus or flavivirus VLP.

In some embodiments of the invention the individual and conformational epitopes of the flavivirus E glycoprotein or dengue virus E glycoprotein can be presented on a synthetic backbone or support structure so that the epitopes within the synthetic backbone or support structure mimic the conformation and arrangement of the epitopes within the structure of the E glycoprotein, virus particle or VLP.

In still further embodiments of the invention, the present invention provides peptide mimitopes (see, Meloen et al. (2000) J. Mol. Recognit. 13:352-359) that mimic the individual and conformational epitopes of the E glycoproteins of the invention. Mimitopes may be identified using any technique known in the art, including but not limited to surface stimulation, random peptide libraries or phage display libraries, as well as an antibody or antibodies to the individual and conformational epitopes of the E glycoproteins of the invention.

The invention further provides a nucleic acid (e.g., an isolated nucleic acid) encoding a chimeric flavivirus VLP or a chimeric flavivirus particle (e.g., a viral coat of the flavivirus particle) of the invention.

Also provided is a cell (e.g., an isolated cell) comprising a vector, a nucleic acid molecule, a dengue virus protein, a dengue virus peptide, a dengue virus protein domain, a flavivirus protein, a flavivirus peptide, flavivirus protein domain, a chimeric dengue virus particle, a chimeric dengue virus VLP, a chimeric flavivirus VLP and/or a chimeric flavivirus particle of this invention, singly or in any combination.

The invention also provides immunogenic compositions comprising the cells, vectors, nucleic acids molecules, dengue virus proteins, chimeric dengue virus VLPs, chimeric dengue virus particles, chimeric flavivirus VLPs and/or chimeric flavivirus particles of the invention, singly or in any combination. In some embodiments, the immunogenic composition is monovalent. In some embodiments, the immunogenic composition is multivalent (e.g., bivalent, trivalent or tetravalent) for dengue virus serotypes DENV1, DENV2, DENV3 and/or DENV4 in any combination. The dengue virus chimeric E glycoproteins of this invention can be administered to a subject singly or in any combination, including any combination of priming and boosting according to such immunization protocols that are known in the art. The dengue virus chimeric E glycoprotein of this invention can be ½, ⅓, ¼, 1/2/3, 1/2/4, 1/3/4, 1/2/3/4, 2/1, 2/3, 2/4, 2/1/3, 2/1/4, 2/3/4, 2/1/3/4, 3/1, 3/2, 3/4, 3/1/2, 3/1/4, 3/2/4, 3/1/2/4, 4/1, 4/2, 4/3, 4/1/3, 4/1/2, 4/3/2, or 4/3/2/1 (wherein the first number of each combination defines the serotype of the backbone and the second, third or fourth number of each combination defines the serotype of the epitope(s) or domain(s) that have been introduced into the backbone). In some embodiments, a dengue virus chimeric E glycoprotein of this invention may comprise one or more substitutions from a serotype which is the same as the serotype of the backbone. In some embodiments, a prime/boost combination would be used that results in administration of antigens representative of all four dengue virus serotypes. Such a prime/boost regimen can include administration of any combination of antigens in any order to achieve this result. A nonlimiting example of a prime/boost protocol can include priming at day 0 and boosting at 3 months and 6 months, or boosting at 6 months and 1 year, respectively. This protocol could also be modified to include only one boost at either 3 months, 6 months or 1 year.

The invention also provides immunogenic compositions comprising the cells, vectors, nucleic acid molecules, VLPs, VRPs, dengue virus or flavivirus particles and/or populations of the invention. The composition can further comprise a pharmaceutically acceptable carrier.

By “pharmaceutically acceptable” it is meant a material that is not toxic or otherwise undesirable, i.e., the material may be administered to a subject without causing any undesirable biological effects. For injection, the carrier will typically be a liquid. For other methods of administration (e.g., such as, but not limited to, administration to the mucous membranes of a subject (e.g., via intranasal administration, buccal administration and/or inhalation)), the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

In some embodiments, the present invention provides a pharmaceutical composition comprising a chimeric dengue virus or flavivirus E glycoprotein, nucleic acid molecule (e.g., an mRNA molecule), vector, VRP, VLP, coronavirus particle, population and/or composition of the present invention, a pharmaceutically acceptable carrier, and, optionally, other medicinal agents, therapeutic agents, pharmaceutical agents, stabilizing agents, buffers, carriers, adjuvants, diluents, etc., which can be included in the composition singly or in any combination and/or ratio.

Immunogenic compositions comprising chimeric dengue virus or flavivirus E glycoprotein, nucleic acid molecule, vector, VRP, VLP, dengue virus or flavivirus particle, population and/or composition of the present invention may be formulated by any means known in the art. Such compositions, especially vaccines, are typically prepared as injectables, either as liquid solutions or suspensions. Solid forms suitable for solution in, or suspension in, liquid prior to injection may also be prepared. Lyophilized preparations are also suitable. In some embodiments, a pharmaceutical composition of the present invention may be a vaccine formulation, e.g., may comprise chimeric dengue virus or flavivirus protein, nucleic acid molecule, vector, VRP, VLP, coronavirus particle, population and/or composition of the present invention and adjuvant(s), optionally in a vaccine diluent. The active immunogenic ingredients are often mixed with excipients and/or carriers that are pharmaceutically acceptable and/or compatible with the active ingredient. Suitable excipients include but are not limited to sterile water, saline, dextrose, glycerol, ethanol, or the like and combinations thereof, as well as stabilizers, e.g., HSA or other suitable proteins and reducing sugars. In addition, if desired, the vaccines or immunogenic compositions may contain minor amounts of auxiliary substances such as wetting and/or emulsifying agents, pH buffering agents, and/or adjuvants that enhance the effectiveness of the vaccine or immunogenic composition.

In some embodiments, a pharmaceutical composition comprising chimeric dengue virus or flavivirus E glycoprotein, nucleic acid molecule, vector, VRP, VLP, dengue virus or flavivirus particle, population and/or composition of the present invention may further comprise additional agents, such as, but not limited to, additional antigen as part of a cocktail in a vaccine, e.g., a multi-component vaccine wherein the vaccine may additionally include peptides, cells, virus, viral peptides, inactivated virus, etc. Thus, in some embodiments, a pharmaceutical composition comprising a chimeric dengue virus or flavivirus E glycoprotein, nucleic acid molecule, vector, VRP, VLP, particle, population and/or composition of the present invention, a pharmaceutically acceptable carrier may further comprise additional viral antigen, e.g., a flavivirus antigen in the form of peptides, peptoids, whole flavivirus (e.g., live attenuated and/or inactivated virus), and/or flavivirus-comprising cells (e.g., cells modified to express flaviviral components, e.g., dengue or other flaviviral peptides).

In some embodiments, a pharmaceutical composition comprising chimeric dengue virus or flavivirus E glycoprotein, nucleic acid molecule, vector, VRP, VLP, particle, population and/or composition of the present invention, and a pharmaceutically acceptable carrier may further comprise an adjuvant. As used herein, “suitable adjuvant” describes an adjuvant capable of being combined with chimeric dengue virus or flavivirus E glycoprotein, nucleic acid molecule, vector, VRP, VLP, particle, population and/or composition of this invention to further enhance an immune response without deleterious effect on the subject or the cell of the subject.

The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

Adjuvants can be combined, either with the compositions of this invention or with other vaccine compositions that can be used in combination with the compositions of this invention.

### Methods

The nucleic acids, proteins, peptides, viruses, vectors, particles, antibodies, VLPs, VRPs, populations, and/or compositions of this invention are intended for use as therapeutic agents and immunological reagents, for example, as antigens, immunogens, vaccines, and/or nucleic acid delivery vehicles. The compositions described herein can be formulated for use as reagents (e.g., to produce antibodies) and/or for administration in a pharmaceutical carrier in accordance with known techniques. See, e.g., Remington, The Science and Practice of Pharmacy (latest edition).

The invention encompasses methods of producing an immune response to a dengue virus in a subject, comprising administering to the subject an effective amount of a dengue virus protein, a chimeric dengue virus particle, a chimeric dengue virus VLP, a chimeric flavivirus VLP, a chimeric flavivirus particle, a nucleic acid molecule, a vector, a cell and/or immunogenic composition of the invention, singly or in any combination.

Further, the present invention can advantageously be practiced to induce an immune response against one, two, three or all four of DENV1, DENV2, DENV3 and DENV4. In some embodiments, the dengue virus chimeric E glycoprotein of this invention and/or a nucleic acid molecule encoding the dengue virus chimeric E glycoprotein of this invention can be administered to a subject singly or in any combination and/or sequence to induce an immune response (e.g., a balanced immune response, in which the parameters of dengue immunity being measured are nearly equivalent for all four DENV serotypes) to all four DENV serotypes. It is well-known in the art that effective and safe multivalent dengue vaccines have been a challenge to design because of the problem of interference among serotypes. For example, the immune response may be predominantly directed against only some of the target serotypes. Multiple vaccinations are then required to try to achieve a response against all serotypes; however, in the case of dengue virus, this approach can be dangerous because repeated administrations to a subject with pre-existing antibodies can lead to dengue hemorrhagic fever.

A still further aspect of the invention is a method of treating a dengue virus infection, comprising administering to the subject an effective amount of a dengue virus protein, a dengue virus protein domain, a dengue virus peptide, a chimeric dengue virus particle, a chimeric dengue virus VLP, a chimeric flavivirus VLP a chimeric flavivirus particle, a nucleic acid molecule, a vector, a cell, and/or immunogenic composition of this invention, singly or in any combination or sequence of combinations.

A still further aspect of the invention is a method of preventing a dengue virus infection, comprising administering to the subject an effective amount of a dengue virus protein, a dengue virus protein domain, a dengue virus peptide, a chimeric dengue virus particle, a chimeric dengue virus VLP, a chimeric flavivirus VLP a chimeric flavivirus particle, a nucleic acid molecule, a vector, a cell, and/or immunogenic composition of this invention, singly or in any combination or sequence of combinations.

A still further aspect of the invention is a method of protecting a subject from the effects of dengue virus infection, comprising administering to the subject an effective amount of a dengue virus protein, a dengue virus protein domain, a dengue virus peptide, a chimeric dengue virus particle, a chimeric dengue virus VLP, a chimeric flavivirus VLP a chimeric flavivirus particle, a nucleic acid molecule, a vector, a cell, and/or immunogenic composition of this invention, singly or in any combination or sequence of combinations.

By “protecting a subject from the effects of dengue virus infection” it is meant that the subject does not develop a disease or disorder caused by a dengue virus infection, or if the subject does develop a disease or disorder caused by a dengue virus infection, the disease or disorder is of less severity and/or symptoms are reduced and/or less severe in the subject in comparison to what the subject would experience upon infection by a dengue virus in the absence of the administration of the dengue virus protein, a dengue virus protein domain, a dengue virus peptide, a chimeric dengue virus particle, a chimeric dengue virus VLP, a chimeric flavivirus VLP a chimeric flavivirus particle, a nucleic acid molecule, a vector, a cell, and/or immunogenic composition of this invention.

The present invention also provides a method of identifying the presence of a neutralizing antibody to dengue virus serotype 1, 2, 3, and/or 4 in a biological sample from a subject, comprising: a) administering a composition comprising an E glycoprotein comprising an E glycoprotein backbone of serotype 1 comprising amino acid substitutions that introduce an epitope that is recognized by an antibody that is reactive with dengue virus serotype 3 and/or a composition comprising an E glycoprotein comprising an E glycoprotein backbone of serotype 3 comprising amino acid substitutions that introduce an epitope that is recognized by an antibody that is reactive with dengue virus serotype 1 and/or a composition comprising an E glycoprotein comprising an E glycoprotein backbone of serotype 2 comprising amino acid substitutions that introduce a dengue virus protein domain of dengue virus serotype 3 and/or a composition comprising an E glycoprotein comprising an E glycoprotein backbone of serotype 3 comprising amino acid substitutions that introduce a dengue virus protein domain of dengue virus serotype 2 to the subject in an amount effective to induce an antibody response to the E glycoprotein; b) contacting a biological sample from the subject with flavivirus particles comprising the E glycoprotein of step (a) above under conditions whereby neutralization of the flavivirus particles can be detected; and c) detecting neutralization in step (b), thereby identifying the presence of a neutralizing antibody to dengue virus serotype 1, 2, 3, and/or 4 in the biological sample from the subject.

In additional embodiments, the present invention provides a method of identifying the presence of a neutralizing antibody to dengue virus serotype 1, 2, 3, and/or 4 in a biological sample from a subject, comprising: a) administering a composition comprising an E glycoprotein comprising an E glycoprotein backbone of serotype 3 comprising amino acid substitutions that introduce an epitope that is recognized by an antibody that is reactive with dengue virus serotype 1 and/or a composition comprising an E glycoprotein comprising an E glycoprotein backbone of serotype 1 comprising amino acid substitutions that introduce an epitope that is recognized by an antibody that is reactive with dengue virus serotype 3 and/or a composition comprising an E glycoprotein comprising an E glycoprotein backbone of serotype 2 comprising amino acid substitutions that introduce a dengue virus protein domain of dengue virus serotype 4 and/or a composition comprising an E glycoprotein comprising an E glycoprotein backbone of serotype 4 comprising amino acid substitutions that introduce a dengue virus protein domain of dengue virus serotype 2 to the subject in an amount effective to induce an antibody response to the E glycoprotein; and b) detecting neutralization in step (a), thereby identifying the presence of a neutralizing antibody to dengue virus serotype 1, 2, 3, and/or 4 in the biological sample from the subject.

The present invention further provides a method of identifying an immunogenic composition that induces a neutralizing antibody to dengue virus serotype 1, 2, 3, and/or 4 in a subject, the method comprising: a) administering a composition comprising an E glycoprotein comprising an E glycoprotein backbone of serotype 3 comprising amino acid substitutions that introduce an epitope that is recognized by an antibody that is reactive with dengue virus serotype 1 and/or a composition comprising an E glycoprotein comprising an E glycoprotein backbone of serotype 1 comprising amino acid substitutions that introduce an epitope that is recognized by an antibody that is reactive with dengue virus serotype 3 and/or a composition comprising an E glycoprotein comprising an E glycoprotein backbone of serotype 4 comprising amino acid substitutions that introduce a dengue virus protein domain of dengue virus serotype 2 and/or a composition comprising an E glycoprotein comprising an E glycoprotein backbone of serotype 2 comprising amino acid substitutions that introduce a dengue virus protein domain of dengue virus serotype 4 to the subject in an amount effective to induce an antibody response to the E glycoprotein; b) contacting a biological sample from the subject with flavivirus particles comprising the E glycoprotein of step (a) under conditions whereby neutralization of the flavivirus particles can be detected; c) determining if the biological sample comprises an antibody that neutralizes flavivirus particles comprising the E glycoprotein of step (a); and d identifying the immunogenic composition as inducing a neutralizing antibody to dengue virus serotype 1, 2, 3, and/or 4 in the subject if the biological sample comprises an antibody that neutralizes flavivirus particles comprising the E glycoprotein of (a).

Additionally provided herein is a method of identifying an immunogenic composition that induces a neutralizing antibody to dengue virus serotype 1, 2, 3, and/or 4 in a subject, the method comprising: a) contacting a biological sample from a subject that has been administered an immunogenic composition comprising an E glycoprotein comprising an E glycoprotein backbone of serotype 3 comprising amino acid substitutions that introduce an epitope that is recognized by an antibody that is reactive with dengue virus serotype 1 and/or a composition comprising an E glycoprotein comprising an E glycoprotein backbone of serotype 1 comprising amino acid substitutions that introduce an epitope that is recognized by an antibody that is reactive with dengue virus serotype 3 and/or a composition comprising an E glycoprotein comprising an E glycoprotein backbone of serotype 2 comprising amino acid substitutions that introduce a dengue virus protein domain of dengue virus serotype 4 and/or a composition comprising an E glycoprotein comprising an E glycoprotein backbone of serotype 4 comprising amino acid substitutions that introduce a dengue virus protein domain of dengue virus serotype 2 with flavivirus particles comprising the E glycoprotein under conditions whereby neutralization of the flavivirus particles can be detected; b) determining if the biological sample comprises an antibody that neutralizes flavivirus particles comprising the E glycoprotein of step (a); and c) identifying the immunogenic composition as inducing a neutralizing antibody to dengue virus serotype 1, 2, 3, and/or 4 in the subject if the biological sample comprises an antibody that neutralizes flavivirus particles comprising the E glycoprotein of (a).

Also provided herein is a method of detecting an antibody to dengue virus serotype 1, 2, 3, and/or 4 in a sample, comprising; a) contacting the sample with a composition comprising an E glycoprotein comprising an E glycoprotein backbone of serotype 3 comprising amino acid substitutions that introduce an epitope that is recognized by an antibody that is reactive with dengue virus serotype 1 and/or a composition comprising an E glycoprotein comprising an E glycoprotein backbone of serotype 1 comprising amino acid substitutions that introduce an epitope that is recognized by an antibody that is reactive with dengue virus serotype 3 and/or a composition comprising an E glycoprotein comprising an E glycoprotein backbone of serotype 2 comprising amino acid substitutions that introduce a dengue virus protein domain of dengue virus serotype 4 and/or a composition comprising an E glycoprotein comprising an E glycoprotein backbone of serotype 4 comprising amino acid substitutions that introduce a dengue virus protein domain of dengue virus serotype 2 under conditions whereby an antigen/antibody complex can form; and b) detecting formation of an antigen/antibody complex, thereby detecting an antibody to dengue virus serotype 1, 2, 3, and/or 4 in the sample.

In yet further embodiments, the present invention provides a method of identifying an antibody to dengue virus serotype 1, 2, 3, and/or 4 in a biological sample from a subject, comprising: a) administering a composition comprising an E glycoprotein comprising an E glycoprotein backbone of serotype 3 comprising amino acid substitutions that introduce an epitope that is recognized by an antibody that is reactive with dengue virus serotype 1 and/or a composition comprising an E glycoprotein comprising an E glycoprotein backbone of serotype 1 comprising amino acid substitutions that introduce an epitope that is recognized by an antibody that is reactive with dengue virus serotype 3 and/or a composition comprising an E glycoprotein comprising an E glycoprotein backbone of serotype 2 comprising amino acid substitutions that introduce a dengue virus protein domain of dengue virus serotype 4 and/or a composition comprising an E glycoprotein comprising an E glycoprotein backbone of serotype 4 comprising amino acid substitutions that introduce a dengue virus protein domain of dengue virus serotype 2 to the subject in an amount effective to induce an antibody response to the E glycoprotein; b) contacting a biological sample from the subject with the E glycoprotein of (a) under conditions whereby an antigen/antibody complex can form; and c) detecting formation of an antigen/antibody complex, thereby identifying an antibody to dengue virus serotype 1, 2, 3, and/or 4 in the biological sample from the subject.

The present invention further provides a method of identifying an antibody to dengue virus serotype 1, 2, 3, and/or 4 in a biological sample from a subject, comprising: a) contacting a biological sample from a subject that has been administered an immunogenic composition comprising an E glycoprotein comprising an E glycoprotein backbone of serotype 3 comprising amino acid substitutions that introduce an epitope that is recognized by an antibody that is reactive with dengue virus serotype 1 and/or a composition comprising an E glycoprotein comprising an E glycoprotein backbone of serotype 1 comprising amino acid substitutions that introduce an epitope that is recognized by an antibody that is reactive with dengue virus serotype and/or a composition comprising an E glycoprotein comprising an E glycoprotein backbone of serotype 2 comprising amino acid substitutions that introduce a dengue virus protein domain of dengue virus serotype 4 and/or a composition comprising an E glycoprotein comprising an E glycoprotein backbone of serotype 4 comprising amino acid substitutions that introduce a dengue virus protein domain of dengue virus serotype 2 with the E glycoprotein under conditions whereby an antigen/antibody complex can form; and b) detecting formation of an antigen/antibody complex, thereby identifying an antibody dengue virus serotype 1, 2, 3, and/or 4 in the biological sample from the subject.

Also provided herein is a method of identifying an immunogenic composition that induces an antibody to dengue virus serotype 1, 2, 3, and/or 4 in a subject, the method comprising: a) contacting a biological sample from a subject that has been administered an immunogenic composition comprising an E glycoprotein comprising an E glycoprotein backbone of serotype 3 comprising amino acid substitutions that introduce an epitope that is recognized by an antibody that is reactive with dengue virus serotype 1 and/or a composition comprising an E glycoprotein comprising an E glycoprotein backbone of serotype 1 comprising amino acid substitutions that introduce an epitope that is recognized by an antibody that is reactive with dengue virus serotype 3 and/or a composition comprising an E glycoprotein comprising an E glycoprotein backbone of serotype 2 comprising amino acid substitutions that introduce a dengue virus protein domain of dengue virus serotype 4 and/or a composition comprising an E glycoprotein comprising an E glycoprotein backbone of serotype 4 comprising amino acid substitutions that introduce a dengue virus protein domain of dengue virus serotype 2 with the E glycoprotein under conditions whereby an antigen/antibody complex can form; and b) detecting formation of an antigen/antibody complex, thereby identifying an immunogenic composition that induces an antibody to dengue virus serotype 1, 2, 3, and/or 4 in the subject.

There are four serotypes of dengue virus (DENV-1, DENV-2, DENV-3 and DENV-4). Within each serotype there are a number of different strains or genotypes. The dengue virus antigens and epitopes of the invention can be derived from any dengue virus, including all serotypes, strains and genotypes, now known or later identified.

In some embodiments of the invention, the dengue virus may be UNC1017 strain (DENV1), West Pacific 74 strain (DENV1), S16803 strain (DENV2), UNC2005 strain (DENV2), UNC3001 strain (DENV3), UNC3043 (DENV3 strain 059.AP-2 from Philippines, 1984), UNC3009 strain (DENV3, D2863, Sri Lanka 1989), UNC3066 (DENV3, strain 1342 from Puerto Rico 1977), CH53489 strain (DENV3), Indonesia 1982 (DENV3), Cuba 2002 (DENV3), UNC4019 strain (DENV4), or TVP-360 (DENV4).

The present invention provides additional non limiting examples of chimeric dengue virus E glycoproteins of this invention that can be used in the compositions and methods described herein in the SEQUENCES section provided herein.

In embodiments of the invention, an “immunogenically active fragment” of a dengue virus polypeptide (e.g., the E protein) comprises, consists essentially of or consists of at least about 6, 8, 10, 12, 15, 20, 30, 50, 75, 100, 125, 150, 200, 250, 300, 350, 400, 450 or more amino acids, optionally contiguous amino acids, and/or less than about 495, 475, 450, 425, 400, 350, 300, 250, 200, 150, 100, 75 or 50 amino acids, optionally contiguous amino acids, including any combination of the foregoing as long as the lower limit is less than the upper limit, and the “immunogenically active fragment” induces an immune response (e.g., IgG and/or IgA that react with the native antigen), optionally a protective immune response, against dengue virus in a host and induces the production of antibodies that specifically bind to the quaternary dengue virus epitope newly identified by the inventors.

The term “epitope” as used herein means a specific amino acid sequence that, when present in the proper conformation, provides a reactive site for an antibody (e.g., B cell epitope) or T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715.

Similarly, conformational epitopes can be readily identified by determining spatial conformation of amino acids such as by, e.g., x-ray crystallography and 2-dimensional nuclear magnetic resonance. Antigenic regions of proteins can also be identified using standard antigenicity and hydropathy plots, such as those calculated using, e.g., the Omiga version 1.0 software program available from the Oxford Molecular Group. This computer program employs the Hopp/Woods method (Hopp et al. Proc. Natl. Acad. Sci USA (1981) 78:3824-3828) for determining antigenicity profiles and the Kyte-Doolittle technique (Kyte et al. J. Mol. Biol. (1982) 157:105-132) for hydropathy plots.

Generally, T-cell epitopes that are involved in stimulating the cellular arm of a subject's immune system are short peptides of about 8-25 amino acids. A common way to identify T-cell epitopes is to use overlapping synthetic peptides and analyze pools of these peptides, or the individual ones, that are recognized by T cells from animals that are immune to the antigen of interest, using, for example, an enzyme-linked immunospot assay (ELISPOT). These overlapping peptides can also be used in other assays such as the stimulation of cytokine release or secretion, or evaluated by constructing major histocompatibility (MHC) tetramers containing the peptide. Such immunogenically active fragments can also be identified based on their ability to stimulate lymphocyte proliferation in response to stimulation by various fragments from the antigen of interest.

The present invention can be practiced for prophylactic, therapeutic and/or diagnostic purposes. In addition, the invention can be practiced to produce antibodies for any purpose, such as diagnostic or research purposes, or for passive immunization by transfer to another subject.

For example, the chimeric E proteins of this invention can be used to immunize a subject against infection by a newly flavivirus, as well as treat a subject infected with a newly emerging flavivirus.

The chimeric E protein of the present invention may be administered in any frequency, amount, and/or route as needed to elicit an effective prophylactic and/or therapeutic effect in a subject (e.g., in a subject in need thereof) as described herein. In certain embodiments, the chimeric E protein, nucleic acid molecule, vector, VRP, VLP, coronavirus particle, population and/or composition is administered/delivered to the subject, e.g., systemically (e.g., intravenously). In particular embodiments, more than one administration (e.g., two, three, four or more administrations) may be employed to achieve the desired level of protein expression over a period of various intervals, e.g., daily, weekly, monthly, yearly, etc. The most suitable route in any given case will depend on the nature and severity of the condition being treated and on the nature of the particular delivery method that is being used. In embodiments wherein a vector is used, the vector will typically be administered in a liquid formulation by direct injection (e.g., stereotactic injection) to the desired region or tissues. In some embodiments, the vector can be delivered via a reservoir and/or pump. In other embodiments, the vector may be provided by topical application to the desired region or by intra-nasal administration of an aerosol formulation. Administration to the eye or into the ear, may be by topical application of liquid droplets. As a further alternative, the vector may be administered as a solid, slow-release formulation. For example, controlled release of parvovirus and AAV vectors is described in international patent publication WO 01/91803, which is incorporated by reference herein for these teachings.

Administration may be by any suitable means, such as intraperitoneally, intramuscularly, intranasally, intravenously, intradermally (e.g., by a gene gun), intrarectally and/or subcutaneously. The compositions herein may be administered via a skin scarification method, and/or transdermally via a patch or liquid. The compositions can be delivered subdermally in the form of a biodegradable material that releases the compositions over a period of time. As further non-limiting examples, the route of administration can be by inhalation (e.g., oral and/or nasal inhalation), oral, buccal (e.g., sublingual), rectal, vaginal, topical (including administration to the airways), intraocular, by parenteral (e.g., intramuscular [e.g., administration to skeletal muscle], intravenous, intra-arterial, intraperitoneal and the like), subcutaneous (including administration into the footpad), intrapleural, intracerebral, intrathecal, intraventricular, intra-aural, intra-ocular (e.g., intra-vitreous, sub-retinal, anterior chamber) and peri-ocular (e.g., sub-Tenon's region) routes or any combination thereof.

In some embodiments, the chimeric E protein can be administered to a subject as a nucleic acid molecule, which can be a naked nucleic acid molecule or a nucleic acid molecule present in a vector (e.g., a delivery vector, which in some embodiments can be a viral vector, such as a VRP). The nucleic acids and vectors of this invention can be administered orally, intranasally, parenterally (e.g., intravenously), by intramuscular injection, by intraperitoneal injection, transdermally, extracorporeally, topically or the like. In the methods described herein which include the administration and uptake of exogenous DNA into the cells of a subject (i.e., gene transduction or transfection), the nucleic acids of the present invention can be in the form of naked DNA or the nucleic acids can be in a vector for delivering the nucleic acids to the cells for expression of the polypeptides and/or fragments of this invention. The vector can be a commercially available preparation or can be constructed in the laboratory according to methods well known in the art.

Delivery of the nucleic acid or vector to cells can be via a variety of mechanisms, including but not limited to recombinant vectors including bacterial, viral, and fungal vectors, liposomal delivery agents, nanoparticles, and gene gun related mechanisms.

In some embodiments, the nucleic acid molecules encoding the E protein(s) of this invention can be part of a recombinant nucleic acid construct comprising any combination of restriction sites and/or functional elements as are well known in the art that facilitate molecular cloning and other recombinant nucleic acid manipulations. Thus, the present invention further provides a recombinant nucleic acid construct comprising a nucleic acid molecule encoding a chimeric E protein of this invention. The nucleic acid molecule encoding the chimeric E protein of this invention can be any nucleic acid molecule that functionally encodes the chimeric E protein of this invention. To functionally encode the chimeric E protein (i.e., allow the nucleic acids to be expressed), the nucleic acid of this invention can include, for example, expression control sequences, such as an origin of replication, a promoter, an enhancer and necessary information processing sites, such as ribosome binding sites, RNA splice sites, polyadenylation sites and transcriptional terminator sequences.

Non-limiting examples of expression control sequences that can be present in a nucleic acid molecule of this invention include promoters derived from metallothionine genes, actin genes, immunoglobulin genes, CMV, SV40, adenovirus, bovine papilloma virus, etc. A nucleic acid molecule encoding a selected chimeric coronavirus S protein can readily be determined based upon the genetic code for the amino acid sequence of the selected polypeptide and/or fragment of interest included in the chimeric coronavirus S protein, and many nucleic acids will encode any selected polypeptide and/or fragment. Modifications in the nucleic acid sequence encoding the polypeptide and/or fragment are also contemplated. Modifications that can be useful are modifications to the sequences controlling expression of the polypeptide and/or fragment to make production of the polypeptide and/or fragment inducible or repressible as controlled by the appropriate inducer or repressor. Such methods are standard in the art. The nucleic acid molecule and/or vector of this invention can be generated by means standard in the art, such as by recombinant nucleic acid techniques and/or by synthetic nucleic acid synthesis or in vitro enzymatic synthesis.

The nucleic acids and/or vectors of this invention can be transferred into a host cell (e.g., a prokaryotic or eukaryotic cell) by well-known methods, which vary depending on the type of cell host. For example, calcium chloride transfection is commonly used for prokaryotic cells, whereas calcium phosphate treatment, transduction, cationic lipid treatment and/or electroporation can be used for other cell hosts.

As another example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega, Madison, WI), as well as other liposomes developed according to procedures standard in the art. In addition, the nucleic acid or vector of this invention can be delivered in vivo by electroporation, the technology for which is available from Genetronics, Inc. (San Diego, CA) as well as by means of a SONOPORATION machine (ImaRx Pharmaceutical Corp., Tucson, AZ).

As another example, vector delivery can be via a viral system, such as a retroviral vector system, which can package a recombinant retroviral genome. The recombinant retrovirus can then be used to infect and thereby deliver to the infected cells nucleic acid encoding the polypeptide and/or fragment of this invention. The exact method of introducing the exogenous nucleic acid into mammalian cells is, of course, not limited to the use of retroviral vectors. Other techniques are widely available for this procedure including the use of adenoviral vectors, alphaviral vectors (e.g., VRPs), adeno-associated viral (AAV) vectors, lentiviral vectors, pseudotyped retroviral vectors and vaccinia viral vectors, as well as any other viral vectors now known or developed in the future. Physical transduction techniques can also be used, such as liposome delivery and receptor-mediated and other endocytosis mechanisms. This invention can be used in conjunction with any of these or other commonly used gene transfer methods.

The present invention further provides a kit comprising one or more compositions of this invention. It would be well understood by one of ordinary skill in the art that the kit of this invention can comprise one or more containers and/or receptacles to hold the reagents (e.g., antibodies, antigens, nucleic acids) of the kit, along with appropriate buffers and/or diluents and/or other solutions and directions for using the kit, as would be well known in the art. Such kits can further comprise adjuvants and/or other immunostimulatory or immunomodulating agents, as are well known in the art.

The compositions and kits of the present invention can also include other medicinal agents, pharmaceutical agents, carriers, diluents, immunostimulatory cytokines, etc. Actual methods of preparing such dosage forms are known, or will be apparent, to those skilled in this art.

Immunomodulatory compounds, such as immunomodulatory chemokines and cytokines (preferably, CTL inductive cytokines) can be administered concurrently to a subject.

Cytokines may be administered by any method known in the art. Exogenous cytokines may be administered to the subject, or alternatively, a nucleic acid encoding a cytokine may be delivered to the subject using a suitable vector, and the cytokine produced in vivo. In particular embodiments, a viral adjuvant expresses the cytokine.

In embodiments of the invention, multiple dosages (e.g., two, three or more) of a composition of the invention can be administered without detectable pathogenicity (e.g., Dengue Shock Syndrome/Dengue Hemorrhagic Fever).

If ex vivo methods are employed, cells or tissues can be removed and maintained outside the body according to standard protocols well known in the art. The nucleic acids and vectors of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

In embodiments of the invention, the multivalent vaccines of the invention do not result in immune interference, e.g., a balanced immune response is induced against all antigens presented. In embodiments of the invention, the balanced response results in protective immunity against DENV-1, DENV-2, DENV-3 and DENV-4.

In embodiments of the invention, the multivalent vaccine can be administered to a subject that has anti-dengue maternal antibodies present.

An adjuvant for use with the present invention, such as any adjuvant disclosed herein, for example, an immunostimulatory cytokine, can be administered before, concurrent with, and/or within a few hours, several hours, and/or 1, 2, 3, 4, 5, 6, 7, 8, 9, and/or 10 days before and/or after the administration of a composition of the invention to a subject.

Furthermore, any combination of adjuvants, such as immunostimulatory cytokines, can be co-administered to the subject before, after and/or concurrent with the administration of an immunogenic composition of the invention. For example, combinations of immunostimulatory cytokines, can consist of two or more immunostimulatory cytokines, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

In embodiments of the invention, the adjuvant comprises an alphavirus adjuvant as described, for example in U.S. Pat. No. 7,862,829.

Boosting dosages can further be administered over a time course of days, weeks, months or years. In chronic infection, initial high doses followed by boosting doses may be advantageous.

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form.

The compositions of the invention can be formulated for administration in a pharmaceutical carrier in accordance with known techniques. See, e.g., Remington, The Science And Practice of Pharmacy (9^{th} Ed. 1995). In the manufacture of a pharmaceutical composition according to the invention, the VLPs are typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R](BASF, Parsippany, N.J.), and the like. These compositions can be sterilized by conventional techniques. The formulations of the invention can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Patent No. 4,501,729. Aerosols of solid particles can likewise be produced with any solid particulate medicament aerosol generator, by techniques known in the pharmaceutical art. Intranasal administration can also be by droplet administration to a nasal surface.

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules, optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles in an inert base such as gelatin and glycerin or sucrose and acacia.

Pharmaceutical formulations suitable for parenteral administration can comprise sterile aqueous and non-aqueous injection solutions, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration are optionally presented as unit dose suppositories. These can be prepared by admixing the active agent with one or more conventional solid carriers, such as for example, cocoa butter and then shaping the resulting mixture.

Pharmaceutical formulations suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of a buffered aqueous solution of the compound(s). Suitable formulations can comprise citrate or bis\tris buffer (pH 6) or ethanol/water and can contain from 0.1 to 0.2M active ingredient.

In embodiments of the invention, the dosage of a virus particle of this invention can be in a range of about 10⁴ to about 10⁷ plaque forming units (PFUs). In embodiments of this invention, the dosage of a VLP of this invention can be in a range of about 500 micrograms to about 5 milligrams. In embodiments of this invention, the dosage of a protein of this invention can be in a range of about 100 to about 10⁴ micrograms+/−adjuvant.

Further, the composition can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

The immunogenic formulations of the invention can optionally be sterile, and can further be provided in a closed pathogen-impermeable container.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1: Dengue Serotype 4/2 Envelope Domain Chimeric Viruses

Dengue is the most prevalent vector-borne virus plaguing our world. In 2010, there were an estimated 390 million infections worldwide, 25% of which were symptomatic. Properties of antibodies that correlate to protection after natural infection or vaccination have not been fully described, which hinders our ability to measure vaccine efficacy. Only two human type-specific neutralizing antibodies against dengue serotype 2 (DENV2) have been mapped.

To identify additional type-specific neutralizing DENV2 epitopes, three chimeric viruses were developed with envelope domain I, II, and III (EDI, EDII, and EDIII) transplants from DENV2 in a DENV4 backbone. Full-length viral RNA of each construct was electroporated into C636 cells and serially passaged in C636, Vero 81, and furin-overexpressing Vero 81 cell lines to compare maturation status and viral titers. Sanger-sequencing confirmed preservation of the DENV2 EDI, EDII, and EDIII footprint, respectively, on the DENV4 backbone. Neutralization assays by DENV2-specific, DENV4-specific, and cross-reactive antibodies showed phenotypic preservation of each DENV2 envelope domain as well. These DENV4-2 chimeric viruses were then used to identify domain-specific epitopes of unmapped monoclonal antibodies. This panel of reagents can be used to identify viral targets by type-specific and polyclonal human sera. An alignment of these DENV 4/2 chimeric E glycoproteins is provided in Table 2. The sequences are provided in the SEQUENCES section, and a table of the substitution positions and a comparison matrix between wildtype DENV4 and DENV2 is shown in Table 4.

### Example 2: Chimeric Dengue Virus Reagents

The four Dengue virus serotypes (DENV1-4) infect several hundred million people per year. Primary DENV infections stimulate serotype-specific (TS) neutralizing antibodies (NAbs) that are correlated with protection against homologous but not heterologous serotypes. A secondary infection with a new serotype stimulates both TS NAbs as well as serotype-cross reactive (CR) NAbs that are associated with long lasting immunity to all serotypes. There is currently a poor understanding of the epitopes targeted by NAbs in people exposed to DENV3 infections or vaccines.

The DENV envelope (E) protein with three distinct domains (EDI, EDII and EDIII) is the main target of NAbs. To study individual variation in humoral immune responses after DENV3 infection or vaccination, this study created a panel of recombinant chimeric DENVs in which the DENV3 EDI, EDII or EDIII domains of the E protein were individually transplanted into a DENV1 backbone. Monoclonals (mAbs) binding to known epitopes on the E-protein were run against DENV1/3 EDI, EDII & EDIII chimeras to assess that each virus retained domain-specific DENV antibody epitopes. These chimeric viruses were used to map three new DENV3 neutralizing MAbs to EDII using the DENV1/3 chimeric set, bringing the total number of known EDII-directed DENV3 TS neutralizing MAbs to 5. Two of the 5 EDII-directed mAbs neutralized DENV3 genotype 3 (GIII) but not genotype 4 (GIV). The critical residues in these epitopes were mapped using DENV3/3 genotype chimeras with select residues or “clusters” from DENV3 GIII transplanted into the DENV3 GIV backbone. Additionally, important structural targets of natural & vaccine-induced polyclonal TS 1° immunity to DENV3 were identified with the DENV1/3 (EDI-III) chimeric panel.

Together, the DENV1/3 EDI-III chimeric virus panel has revealed major antigenic sites on E protein targeted by NAbs in people exposed to primary DENV3 infections. Full-domain transplant chimeric reagents can be used as a tool to assess antibody quality after natural infection or vaccination & to learn about structural targets of immunity for other DENV serotypes. An alignment of these chimeric E glycoproteins is provided in Table 1. The sequences are provided in the SEQUENCES section, and a table of the substitution positions and a comparison matrix between wildtype DENV1 and DENV3 is shown in Table 3.

### Example 3: Design and Recovery of DENV1/3 EDII and EDIII Recombinant Viruses

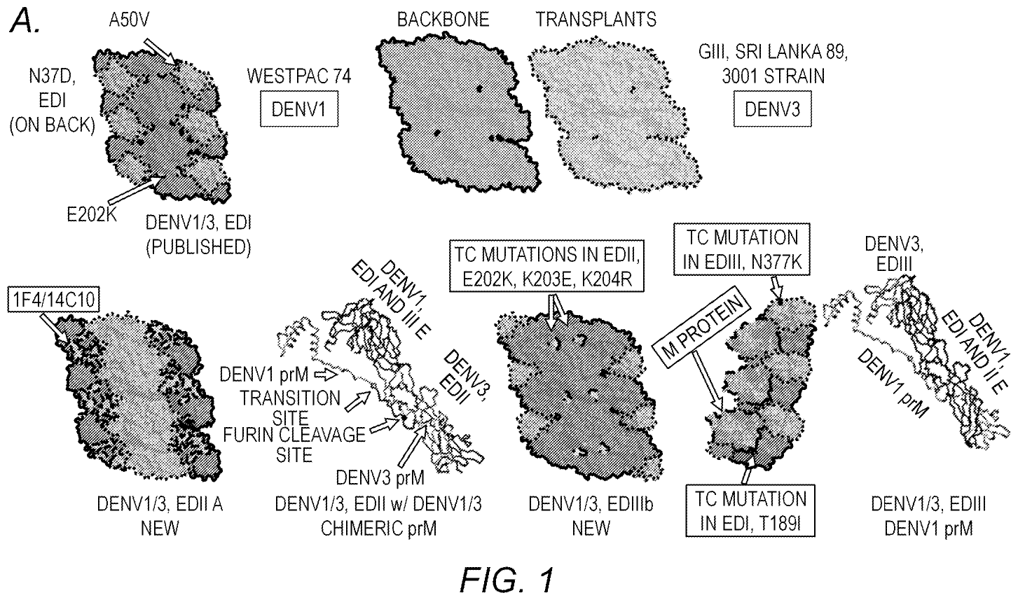

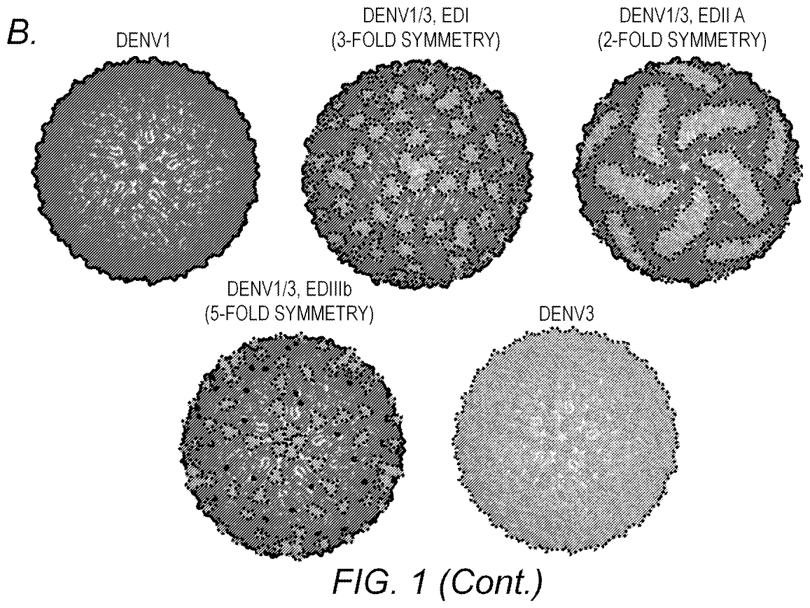

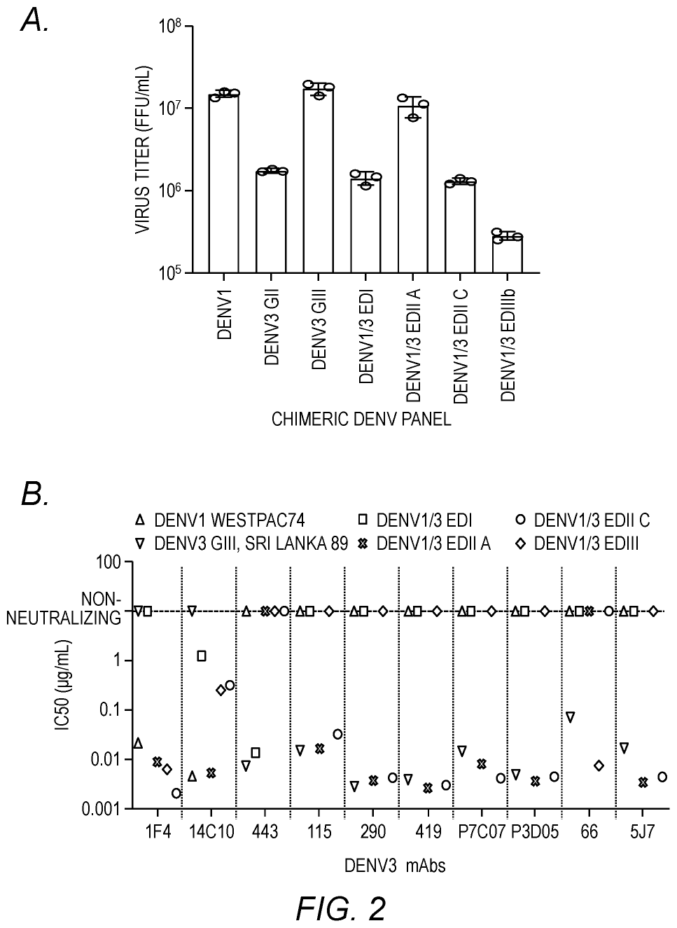

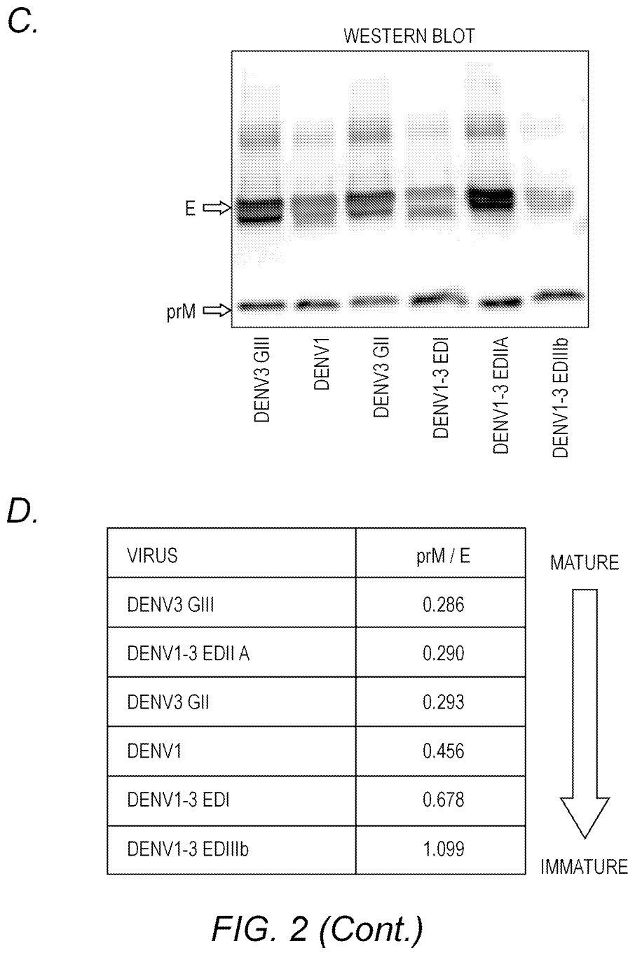

A DENV1/3 full ED chimeric panel was created to feature the EDI, EDII or EDIII residues from the DENV3, G-III, Sri Lanka ′89 strain introduced into the DENV1 (Westpac) E glycoprotein (FIG. 1 panels A and B; Table 5). This study designed DENV1/3 EDII and EDIII recombinant virus chimeras that encode the DENV3 E glycoprotein 2-fold and 5-fold symmetry axes, respectively. Initial attempts to recover a recombinant DENV1 virus that encoded the DENV3 EDII domain were not viable. Rather, viable DENV1/3 EDII chimeras required the inclusion of a serotype-matched chimeric prM, designed to maintain favorable steric prM-E interface interactions that likely function in maturation and release. Specifically, residues 1-108 of the DENV3 prM were fused with residues 109-166 on the DENV1 M protein, thereby preserving N and C-terminal interactions with the appropriate homologous DENV1 and DENV3 E protein residues (Table 6). Four DENV1/3 EDII constructs (A-D) were designed and recovered, with between 24 and 38 amino acid residues transplanted from DENV3 into DENV1, with DENV1/3 EDIIC and D encoding increasing numbers of DENV3 residues in the EDI-EDII and EDI-EDIII hinge regions (Table 7). Of the 4 EDII chimeras, DENV1/3 EDIIA replicated most efficiently to 1×10⁷ FFU/mL titer (FIG. 2 panel A) and encoded 33 DENV3 residues that preserved DENV1 hmABs' 14C10 and 1F4 EDI and EDIII dominant epitopes. Replicating to titers approaching 1.3×10⁶FFU, DENV1/3 EDIIC encoded 37 DENV3 residues and presented fully intact 1F4 and partially intact 14C10 epitopes, respectively. DENV1/3 EDIIB and D, encoding 24 and 38 DENV3 residues, were also recovered, but replicated inefficiently.

Four DENV1/3 EDIII mutants were constructed (a-d), designed to preserve DENV1 EDIII residues on the interaction interfaces and select EDIII residues engaging the M and capsid proteins during virion assembly (Table 8). All constructs were recovered on C636 cells but had attenuated growth in Vero81 NHP cells. The most robust mutant, DENV1/3 EDIIIb, retained one DENV1 EDIII residue at position F392 and replicated to titers of 2.8×10⁵ FFU/mL on Vero81 cells. During adaptation, tissue culture (TC) mutations arose at position N377K, a subsurface residue in EDIII, and at positions E202K, K203E and K204R in EDII, which are localized along the interface between two monomers in the dimer, and one mutation in EDI, T189I, another subsurface residue interacting with M (FIG. 1 panel A). DENV1/3 EDIIIc was less hardy and DENV1/3 EDIIIa and EDIIId could not be recovered in Vero81 cells. The most replication competent chimeras (DENV1/3 EDIIA, DENV1/3 EDIIC and DENV1/3 EDIIIb,) were selected for downstream immunologic and biochemical phenotyping.

Monoclonal Antibody Neutralization Phenotypes within the DENVI 3 Full Panel: 13 DENV3 TS human monoclonal antibodies (hmAbs) have been previously mapped to EDI, EDII, and EDII To evaluate the E glycoprotein antigenic profiles in the recombinant panel, a 20 panel of DENV3 hmAbs (-443, -115, -290, -419-66, -5J7, -P3D05 and -P7C07) isolated after natural infection or natural infection, then tetravalent vaccination (Young et al. 2020 Cell Host Microbe 27:710-724), and two DENV1 hmAbs (-1F4 and -14C10) were evaluated by focus reduction neutralization test (FRNT) with the WT parental strains and the DENV1/3 EDI, EDIIA, EDIIC and EDIIIb chimeric panel (FIG. 2 panel B). As designed, anti-EDI-anchored DENV1 TS hmAb-1F4 neutralization was preserved in DENV1 and the DENV1/3 EDIIA, EDIIC and EDIIIb chimeras, but not in DENV3 or in DENV1/3 EDI chimeras (Fibriansah et al. 2014 EMBO Mol Med 6:358-371). The anti-EDI/IJI domain spanning DENV1 TS hmAb-14C10 potently neutralized DENV1/3 EDIIA and DENV3, but lost ≥56% of its neutralizing activity against the DENV1/3 EDI, EDIIC and EDIIIb recombinants. Loss of neutralization in DENV1/3 EDIIC was likely due to the introduction of DENV3 residues N52Q and T275G into the 14C10 epitope footprint. Several natural infection and vaccine derived hmAbs (e.g., 115, -290, -419, -5J7, -P3D05 and -P7C07) potently neutralized the DENV1/3 EDIIA and EDIIC chimeras and DENV3, but not the other ED chimeras, localizing those antibodies to DENV3 EDII. Finally, the DENV3 EDIII targeting TS hmAb-66 efficiently neutralized the DENV1/3 EDIIIb chimera and DENV3, but not the remainder of the chimeric viruses.

To evaluate maturation status, Western blot analysis demonstrated that DENV1/3 EDIIIb is more immature than DENV1 WT (prM/E ratios 1.099 and 0.456 respectively), potentially suggesting that the TS hmAB-66 may preferentially target immature virions (FIG. 2 panels C and D). Each DENV3 hmAb neutralized only the chimera containing its epitope's domain, but not the other chimeras, confirming overall ED epitope presentation across the DENV1/3 panel. Based on virologic and immunologic phenotyping with hmABs, DENV1/3 EDIIA was chosen as the best candidate for DENV1/3 EDII domain-specific mapping.

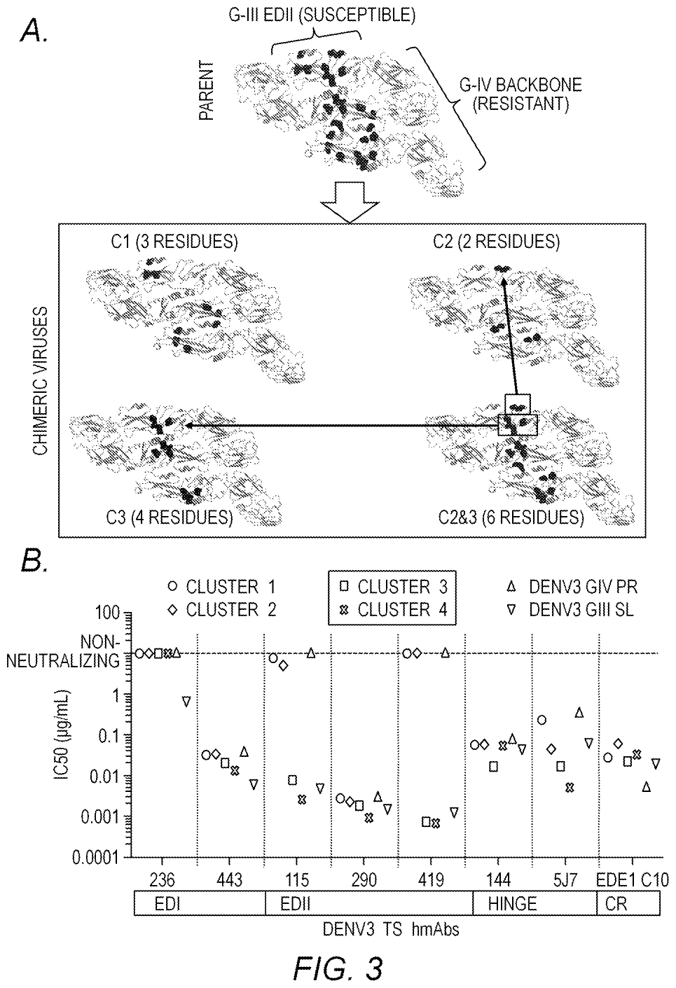

Evidence for Multiple Unique Overlapping Neutralizing Epitopes in DENV3 EDH: Previous studies have suggested that DENV3 neutralizing hmAbs -115 and -419 likely targeted DENV3 EDII and were disrupted by natural genotypic variation (Young et al. 2020). A third EDII DENV3 TS hmAb, -290, did not exhibit differential neutralization across a panel of DENV3 genotypes. To functionally map the exact EDII residues that regulated hmAB-115 and -419 genotype specific neutralization, a panel of EDII mutants (DENV3/3, C₁-C₄) were created by inserting susceptible G-III residues into the G-IV resistant backbone (Table 9). As these 9 residues were spatially clustered on the E glycoprotein dimer (FIG. 3 panel A), proximal blocks of 3 or 6 DENV3 G-III, EDII residues were transplanted into the resistant G-IV backbone (e.g., C1: T68I, T81V and S113L; C4: G62E, A63G, H120Q, L124P, A224T and V226T). In parallel, two additional recombinants were designed which further subdivided the six residues in C4 into two smaller clusters (C2: A224T, V226T; C3: G62E, A63G, H120Q and L124P). All viruses were viable and replicated efficiently in Vero81 NHP cells. DENV3 genotype mutants C3 and C4 and the DENV3, G-III WT, but not DENV3 C1, C2 or DENV3 G-IV WT, were potently neutralized by hmAbs-115 and -419. These data demonstrate that C3 contains important functional residues for hmAB-115 and -419 neutralization (e.g., G62E, A63G, H120Q and L124P) (FIG. 3 panels B and C). However, primary DENV3 naturally infected, then secondary tetravalent vaccine-derived DENV3 EDII hmAbs-P7C07 and -P3D05 efficiently neutralized all DENV3 genotypic variants in the panel (FIG. 3 panel D). These data suggest that hmABs -P7C07 and -P3D05 either recognize novel EDII epitopes or engage an overlapping epitope in a unique manner.

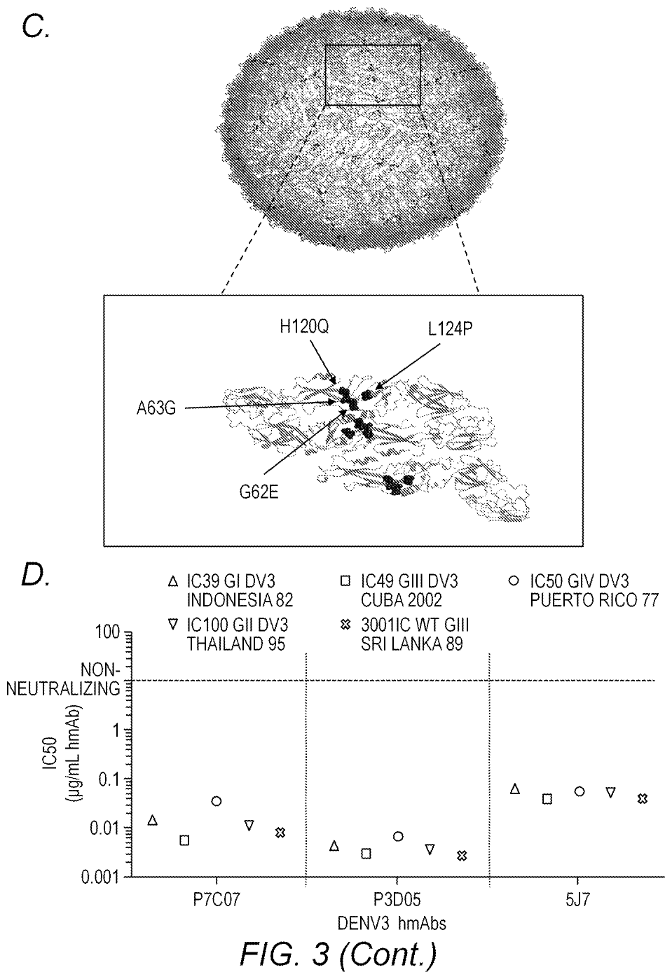

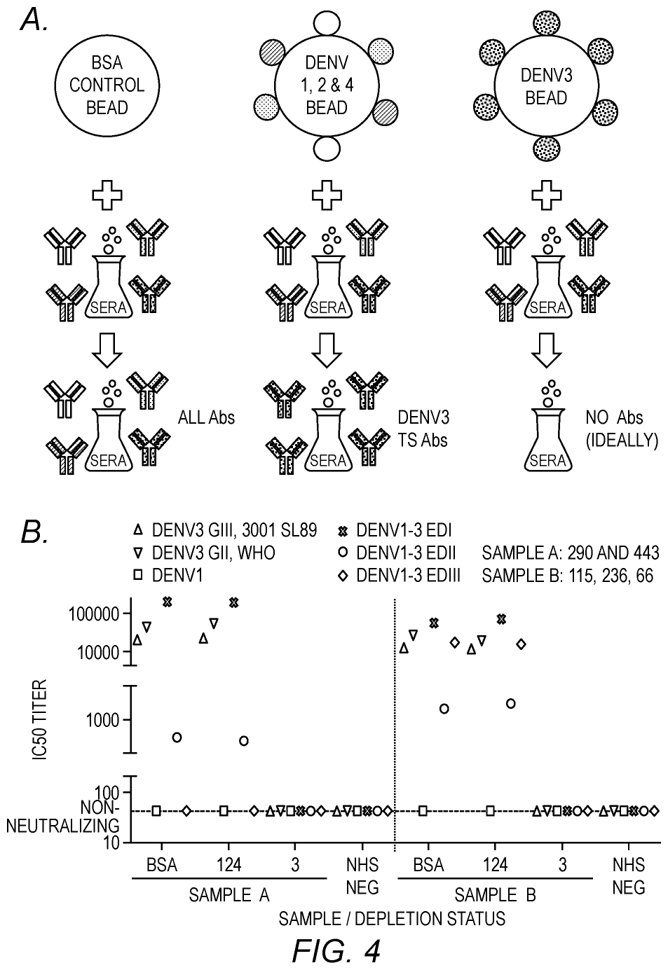

Strategy for Mapping DENV3 E Glycoprotein Domain Specific Neutralizing Phenotypes in Polyclonal Sera: To define E-glycoprotein domain-specific structural targets of the DENV3 TS human polyclonal antibody response, DENV3 primary convalescent or vaccinated human serum samples were first depleted of CR or TS antibodies or control depleted using beads coated with either a bovine serum albumin (BSA) control, a combination DENV1, 2 and 4 (heterotypic depletion), or with a WHO DENV3 G-II reference strain (homotypic depletion) (Patel et al. 2017 PLoS Negl Trop Dis 11:e0005554; FIG. 4 panel A). To demonstrate the efficiency of depletion studies, DENV3 EDI, EDII and EDIII directed TS hmAbs were introduced into heat-inactivated normal human sera (NHS), depleted using the three conditions above, and then assayed by FRNT. Sample A was comprised of hmAbs-290 (EDII) and -443 (EDI) while Sample B contained hmAbs-236 (EDI), -115 (EDII) and -66 (EDIII). Under these conditions, little, if any, reductions in neutralization titers were noted after applying the control and DENV1,2,4 depletions. Under identical conditions, all DENV3 hmAb neutralization titers were lost after DENV3 homotypic depletion (FIG. 4 panel B).

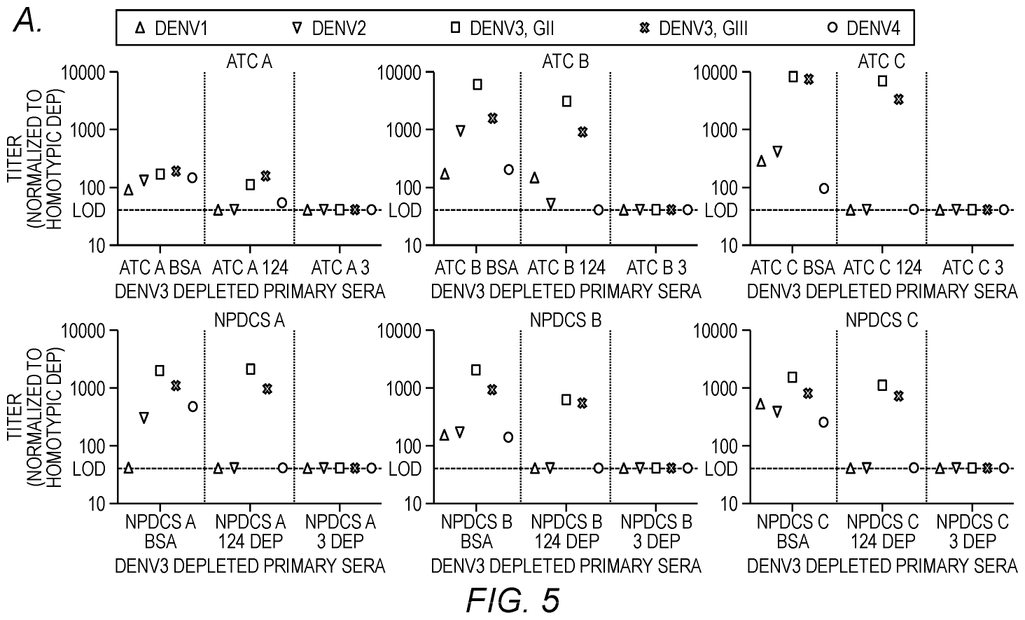

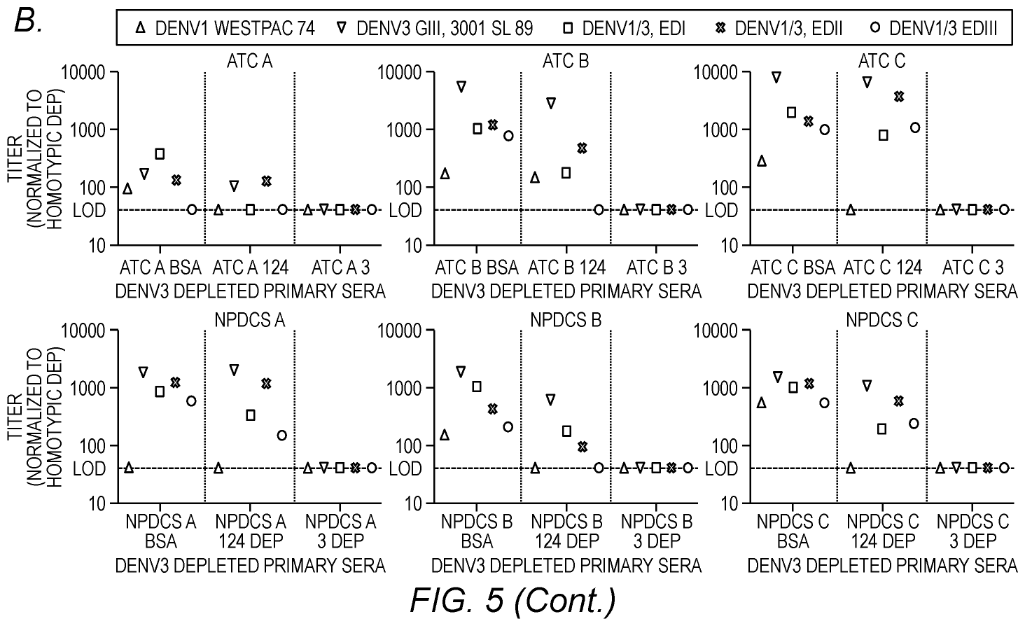

DENV3 ED Neutralizing Antibody Responses after Primary Natural Infection: Primary human DENV3 (G-III) natural infection serum samples were obtained from the Nicaraguan Pediatric Dengue Cohort Study (NPDCS)(n=3) and the Adult Arbovirus Traveler Cohort (n=3) (Table 10). After depletion, neutralization titers were evaluated using the DENV1/3 EDI-III panel, DENV1 and DENV3 G-III, as well as DENV2, DENV4 and DENV3 G-II strains. When tested with both DENV3 G-II and G-III, there was very little difference in ID50 DENV3 TS neutralization titers in each of the patient samples (geometric mean fold change=2), even though the serum was obtained from convalescent patients who experienced a DENV3 G-III primary infection (FIG. 5 panel A) After heterotypic depletion with DENV1, 2, and 4, the primary DENV3 TS neutralization response either preferentially (5/6) or significantly (1/6) neutralized DENV1/3 EDIIA, suggesting that EDII is the dominant target of DENV3 TS sera after natural infection (FIG. 5 panel B). However, variations in the response were also noted in the population. For example, neutralization titers from one subject from Nicaragua targeted DENV1/3 EDI over DENV1/3 EDIIA, though at similar levels (ID50=173 vs 99). Convalescent serum samples generally targeted neutralization epitopes encoded in EDI (5/6) and EDIII (3/6), respectively, though more weakly than EDII.

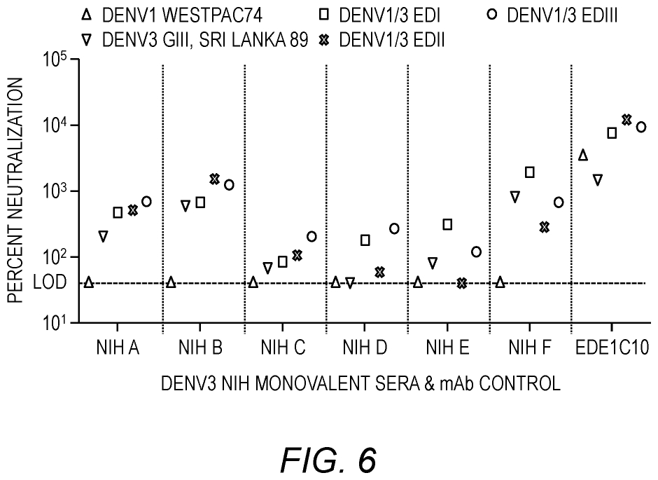

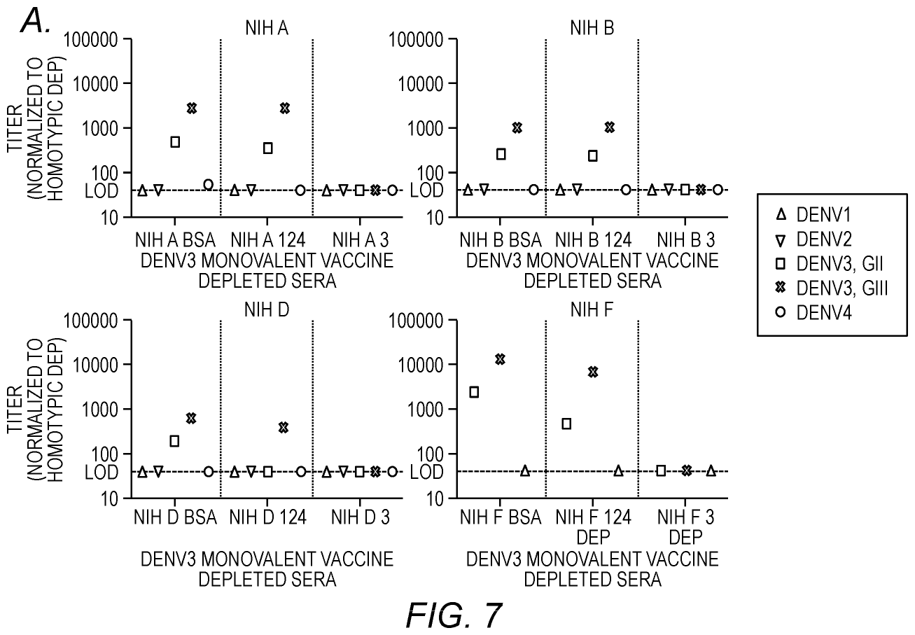

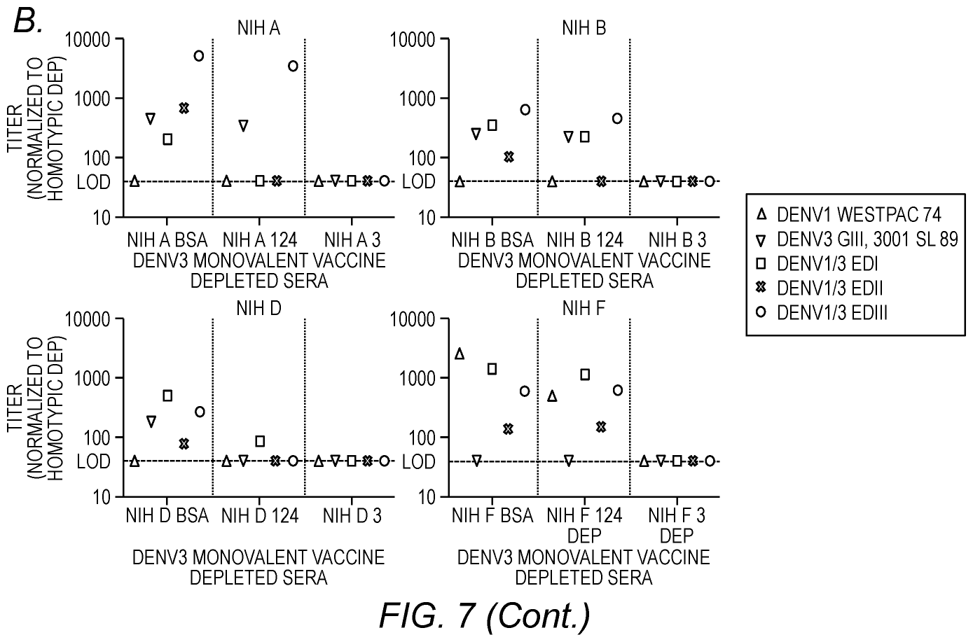

DENV3 ED Specific Neutralization Responses following Monovalent Vaccination: Next, Six DENV3 (G-I) NIH monovalent vaccine serum samples were analyzed at 180 days after prime/boost (Table 10). Using undepleted serum samples, variable neutralization titers were noted across the DENV1/3 panel (FIG. 6). Although highly variable, most undepleted serum samples neutralized all 3 chimeras (5/6 samples). To measure DENV3 TS responses, four of the highest-titered samples were depleted. When tested for neutralization of DENV3 G-II and G-III, DENV3 G-II (geometric mean fold change=10) was more efficiently neutralized by DENV3 TS vaccine serum than DENV3 G-II in all cases (FIG. 7 panel A). Surprisingly, only one out of four depleted NIH DENV3 monovalent TS serum samples structurally targeted DENV1/3 EDIIA, but at a much lower level than DENV1/3 EDI and EDIIIb (FIG. 7 panel B). In contrast, EDI and/or EDIII neutralizing epitopes were preferentially targeted in all vaccine recipients tested. Two of the 4 DENV3 TS serum samples efficiently neutralized DENV1/3 EDI and DENV1/3 EDIIIb at similar titers. Among the remaining DENV3 TS samples, one patient sample only neutralized DENV1/3 EDI while another only neutralized DENV1/3 EDIII. In contrast to natural infection sera, these data suggest that NIH DENV3 monovalent vaccination preferentially induces DENV3 TS antibodies that target EDI and EDIII. Together, these data reveal significant differences between the types of antibodies derived after natural infection and vaccination.

Dissecting the Percentage of TS and CR anti-DENV Antibodies in Polyclonal Serum Samples: Pairing the use of domain-specific recombinant viruses and depletion studies, the percentage of TS and CR antibodies in each serum sample after natural infection or vaccination was calculated. After natural infection (n=6), the polyclonal serum antibody population that targeted DENV1/3 EDIIA was generally more TS (mean=67%) than the responses targeting DENV1/3 EDI or EDIIIb (mean=22% and 35%, respectively) (Table 11). Conversely, overall, the NIH DENV3 monovalent vaccine samples had a minimal TS response (mean=25%) to DENV1/3 EDIIA, but higher responses to DENV1/3 EDI (mean=42%) and EDIIIb (mean=68%) (Table 12). After natural infection, one individual (ATC C) had equivalent % TS polyclonal responses to DENV1/3 EDIIA and EDIIIb, although maximal ID50 titer was much higher for EDIIA than EDIIIb (ID50=3827 vs 1104, respectively). Conversely, one NIH monovalent vaccine sample (NIH F) had 100% TS antibodies targeting EDIIA, however the overall level of antibody was much lower towards EDIIA than EDIIIb (ID50=149 vs 642, respectively). In the NIH monovalent vaccine sera, the % TS responses were higher to DENV3 G-II than G-III. These data support earlier hypotheses that vaccine neutralizing antibody responses are more impacted by genotypic variation within a serotype as compared to natural infection.

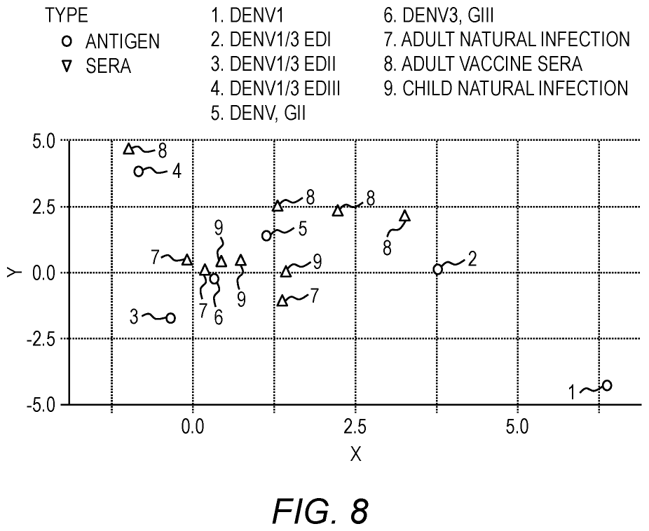

Visualizing the Antigenic Relationships Between Viruses and Polyclonal Sera: To better visualize the domain-specific antigenic relationships, antigenic cartography was employed, plotting differences in the neutralizing polyclonal response between viruses in 2-dimensional space (FIG. 8). After natural infection, neutralizing responses mapped closest to DENV3 G-III, then DENV3 G-II strains, and were in close proximity to DENV1/3 EDIIA, indicating that antigenic juxtaposition was heavily regulated by polyclonal responses preferentially targeting DENV3 EDII encoded epitopes. Within the convalescent serum population (n=6), there were also two clear phenotypes. The NPDCS natural children's sera (n=3) are positioned near equidistant between DENV3 G-II and G-III. While the ATC adult natural sera (n=3) grouped closer to DENV3 G-III than G-II. DENV1/3 EDI and EDIIIb were more antigenically distant from both genotypes of DENV3 than DENV1/3 EDIIA, with EDI sequences contributing more skew towards DENV1 than the EDIIIb virus, which mapped the furthest from DENV1. In contrast to natural infection serum responses, NIH monovalent sera mapped more closely to DENV3 G-II than G-III and to DENV3 EDI and EDIIIb in the DENV1/3 chimeras, but not DENV1/3 EDIIA. These data clearly demonstrate strong antigenic shifts in ED epitope targeting between natural and NIH vaccine sera.

### Example 4: Engineering Chimeric DENV4/2 Envelope Domain Panel to Compare Targets of 12 DENV2 Neutralizing Antibodies in Younger and Older Children

Among children, age differences in vaccine efficacy and disease severity have been documented in controlled trials and observational population studies. Neutralizing antibodies are critical determinants of protection against Dengue virus (DENV2) infection. In order to identify differences in neutralizing antibody potency and epitopes among primary DENV2 infected individuals, we are developing a panel of chimeric DENV4-2 viruses that isolate DENV2 envelope domain 1, 2, and 3 on a DENV4 backbone(DENV4-2 EDI, EDII, EDIII, respectively). All viruses were designed using a four-piece reverse genetics system and electroporated into C6/36 cells and passaged in Vero81 cells. A reduction in growth kinetics in Vero81 compared to C6/36 cells was observed. Maturity of the chimeras reflected maturity of the parent virus. The chimeric viruses retained the predicted parental antigenic epitopes when validated with a panel of DENV2-specific, DENV4-specific, and cross-reactive antibodies. In conclusion, this panel of viruses can be used to describe determinants of protection in different populations or identify epitopes of neutralizing monoclonal antibodies. We are currently using this viral panel to investigate differences in domain-specific neutralizing antibodies against DENV2 after primary natural infection in toddlers and pre-teens.

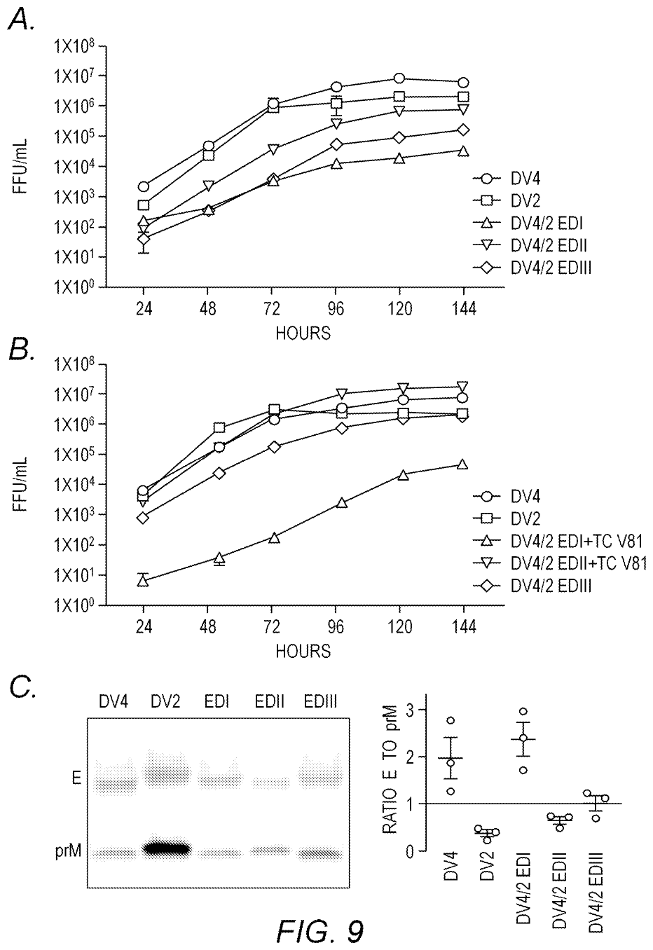

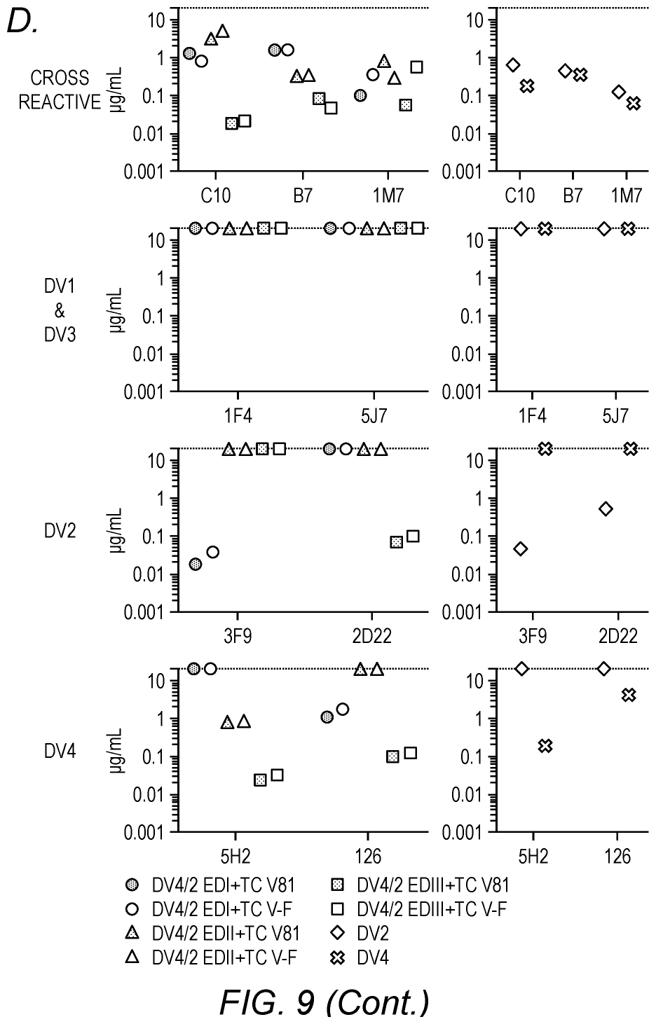

Results of this study are shown in FIG. 9 panels A-D. Three recombinant chimeric viruses were generated where the dengue virus serotype 2 (DV2) envelope domain (ED) I, II, and III region was inserted into the backbone sequence of a DV4 molecular clone, cloned using a four-piece reverse genetics system into C6/36 and Vero cell lines.

Viral recovery and sequence verification: DV4/2 EDI and DV4/2 EDII acquired 3 and 2 tissue culture adaptations in E, respectively, and reconstructed viruses DV4/2 EDI+TC and D4/2 EDII+TC to include those single residue mutations grew to higher titers on mammalian cells compared to original construct (FIG. 9 panels A and B). While not wishing to be bound to theory, the acquired adaptations may be involved in the stability of the mature envelope and fusion formation.

Characterization of maturation state: The prM region on immature dengue particles are targets of short-lived cross-reactive antibodies, thus a mature viral stock is best suited for assessing type-specific antibodies. Staining for prM and E by Western found that DV4 and DV4/2 EDI were more mature than DV2, DV4/2 EDII, and DV4/2 EDIII. The viral panel was passaged in furin-overexpressing Vero (V-F) cells to ensure mature viral stocks.

FRNT with described monoclonal antibodies: To confirm the preservation of DV2 envelope domain epitope, FRNT with cross-reactive, DV1 and DV3 TS mAbs, DV2, and DV4 mAbs was conducted. DV2 TS mAbs 3F9 and 2D22 neutralized DV4/2 EDI and DV4/2 EDIII, respectively, while DV4 TS mAb 5H2 neutralized DV4/2 EDII and DV4/2 EDIII and 126 neutralized DV4/2 EDI and DV4/2 EDIII but not DV4/2 EDII.

The DV4/2 envelope-domain chimeric recombinant viruses preserve the DV2 EDI, EDII, and EDIII regions, respectively. This panel may be used to assess envelope-domain targets of human polyclonal sera after natural infection and vaccination. Completion of this panel will allow mapping of monoclonal antibodies and epitopes targeted by polyclonal human sera across all three domains of the DV2 E protein.

SEQUENCES

DENV 1/3 EDII “small” 438-2 chimera prM (SEQ ID NO: 7) + E (SEQ ID NO: 1)
FHLTSRDGEPRMIVGKNERGKSLLFKTASGINMCTLIAMDLGEMCDDTLTYKCPH
ITEVEPEDIDCWCNLTSTWVTYGTCNQAGEHRRDKRSVALAPHVGMGLDTRTQTWMS
SEGAWKQIQKVETWALRHPGFTVIALFLAHAIGTSITQKGIIFILLMLVTPSMAMRCVGI
GNRDFVEGLSGATWVDVVLEHGSCVTTMAKDKPTLDIELLKTEVTNLATLRKLCI
EGKITNITTDSRCPTQGEAVLPEEQDQNYVCKHTYVDRGWGNGCGLFGKGSLVTC
AKFQCLEPIEGKVVQYENLKYSVIVTVHTGDQHQVGNETTEHGTIATITPQAPTSEI
QLTDYGALTLDCSPRTGLDFNEMILLTMKNKAWMVHRQWFFDLPLPWTSGATTE
TPTWNRKELLVTFKNAHAKKQEVVVLGSQEGAMHTALTGATEIQNSGTTTIFAGH
LKCRLKMDKLTLKGMSYVMCTGSFKLEKEVAETQHGTVLVQIKYEGTDAPCKIP
FSTQDEKGVTQNGRLITANPIVTDKEKPVNIEAEPPFGESYIVIGAGEKALKLSWFK
KGSSIGKMFEATARGARRMAILGDTAWDFGSIGGVFTSVGKLVHQIFGTAYGVLF
SGVSWTMKIGIGVLLTWLGLNSRSTSLSMTCIAVGLVTLYLGVMVQA
DENV 1/3 EDII “large” 438-3 chimera prM (SEQ ID NO: 7) + E (SEQ ID NO: 2)
FHLTSRDGEPRMIVGKNERGKSLLFKTASGINMCTLIAMDLGEMCDDTLTYKCPH
ITEVEPEDIDCWCNLTSTWVTYGTCNQAGEHRRDKRSVALAPHVGMGLDTRTQTWMS
SEGAWKQIQKVETWALRHPGFTVIALFLAHAIGTSITQKGIIFILLMLVTPSMAMRCVGI
GNRDFVEGLSGATWVDVVLEHGSCVTTMAKDKPTLDIELLKTEATQLATLRKLCI
EGKITNITTDSRCPTQGEAVLPEEQDQNYVCKHTYVDRGWGNGCGLFGKGSLVTC
AKFQCLEPIEGKVVQYENLKYSVIVTVHTGDQHQVGNETTEHGTIATITPQAPTSEI
QLTDYGALTLDCSPRTGLDFNEMILLTMKNKAWMVHRQWFFDLPLPWTSGATTE
TPTWNRKELLVTFKNAHAKKQEVVVLGSQEGAMHTALTGATEIQNSGGTSIFAGH
LKCRLKMDKLTLKGMSYVMCTGSFKLEKEVAETQHGTVLVQIKYEGTDAPCKIP
FSTQDEKGVTQNGRLITANPIVTDKEKPVNIEAEPPFGESYIVIGAGEKALKLSWFK
KGSSIGKMFEATARGARRMAILGDTAWDFGSIGGVFTSVGKLVHQIFGTAYGVLF
SGVSWTMKIGIGVLLTWLGLNSRSTSLSMTCIAVGLVTLYLGVMVQA
DENV1/3, EDIII chimera prM (SEQ ID NO: 8) + E (SEQ ID NO: 3)
FHLTTRGGEPHMIVSKQERGKSLLFKTSAGVNMCTLIAMDLGELCEDTMTYKCPRITE
TEPDDVDCWCNATETWVTYGTCSQTGEHRRDKRSVALAPHVGLGLETRTETWMSSEG
AWKQIQKVETWALRHPGFTVIALFLAHAIGTSITQKGIIFILLMLVTPSMAMRCVGIGN
RDFVEGLSGATWVDVVLEHGSCVTTMAKDKPTLDIELLKTEVTNPAVLRKLCIEA
KISNTTTDSRCPTQGEATLVEEQDTNFVCRRTFVDRGWGNGCGLFGKGSLITCAK
FKCVTKLEGKIVQYENLKYSVIVTVHTGDQHQVGNETTEHGTIATITPQAPTSEIQL
TDYGALTLDCSPRTGLDFNEMVLLTMKKKSWLVHKQWFLDLPLPWTSGASTSQE
TWNRQDLLVTFKTAHAKKQEVVVLGSQEGAMHTALTGATEIQTSGTTTIFAGHL
KCRLKMDKLTLKGMSYAMCTNTFVLKKEVSETQHGTILIKVEYKGEDAPCKIPFS
TEDGQGKAHNGRLITANPVVTKKEEPVNIEAEPPFGESNIVIGIGDNQHTGGRFKK
GSSIGKMFEATARGARRMAILGDTAWDFGSVGGVLNSLGKMVHQIFGSAYTALFS
GVSWVMKIGIGVLLTWIGLNSKNTSMSFSCIAIGIITLYLGPYFAT
DENV4/2 EDI prM (SEQ ID NO: 9) + E (SEQ ID NO: 4)
FHLSTRDGEPLMIVAKHERGRPLLFKTTEGINKCTLIAMDLGEMCEDTVTYKCPLLVNT
EPEDIDCWCNLTSTWVMYGTCTQSGERRREKRSVALTPHSGMGLETRAETWMSSEGA
WKHAQRVESWILRNPGFALLAGFMAYMIGQTGIQRTVFFVLMMLVAPSYGMRCIGIS
NRDFVEGVSGGSWVDIVLEHGSCVTTMAQNKPTLDFELIKTEAKEVALLRTYCIEA
SISNITTATRCPTQGEPYLKEEQDQQYICRRDVVDRGWGNGCGLFGKGGVVTCAK
FSCSGKITGNLVQPENLEYTIVVTPHSGEEHAVGNDTGKHGKEIKVTPQSSITEAEL
TGYGTVTMECSPRTGLDFNEMILMKMKKKTWLVHKQWFLDLPLPWTAGADTSE
VHWNYKERMVTFKVPHAKRQDVTVLGSQEGAMHSALAGATEVDSGDGNHLFAG
HLKCRLRMDKLQLKGMSYTMCSGKFKIDKEMAETQHGTTVVKVKYEGAGAPCK
VPIEIRDVNKEKVVGRVISSTPLAENTNSVTNIELEPPFGDSYIVIGVGNSALTLHWF
RKGSSIGKMFESTYRGAKRMAILGETAWDFGSVGGLFTSLGKAVHQVFGSVYTT
MFGGVSWMIRILIGFLVLWIGTNSRNTSMAMTCIAVGGITLFLGFTVQA
DENV4/2 EDII prM (SEQ ID NO: 10) + E (SEQ ID NO: 5)
FHLTTRNGEPHMIVSRQEKGKSLLFKTEDGVNMCTLMAMDLGELCEDTITYNCPLLRQ
NEPEDIDCWCNSTSTWVTYGTCTTTGEHRRGKRSVALVPHVGMGLETRTETWMSSEG
AWKHAQRVESWILRNPGFALLAGFMAYMIGQTGIQRTVFFVLMMLVAPSYGMRCVG
VGNRDFVEGVSGGAWVDLVLEHGGCVTTMAQGKPTLDFELIKTEAKQPATLRKY
CIEAKLTNTTTESRCPTQGEPSLNEEQDKRFVCKHSMVDRGWGNGCGLFGKGGIV
TCAMFTCKKNMEGKVVQPENLEYTVVVTVHNGDTHAVGNDTSNHGVTATITPRS
PSVEVKLPGYGELTLDCEPRSGLDFNEMVLLQMENKAWLVHRQWFLDLPLPWLP
GADTQGSNWIQKETLVTFKNPHAKKQDVVVLGSQEGAMHTALTGATEIQMSSGN
LLFTGHLKCKVRMEKLRIKGMSYTMCSGKFSIDKEMAETQHGTTVVKVKYEGAG
APCKVPIEIRDVNKEKVVGRVISSTPLAENTNSVTNIELEPPFGDSYIVIGVGNSALTL
HWFRKGSSIGKMFESTYRGAKRMAILGETAWDFGSVGGLFTSLGKAVHQVFGSV
YTTMFGGVSWMIRILIGFLVLWIGTNSRNTSMAMTCIAVGGITLFLGFTVQA
DENV4/2 EDIII prM (SEQ ID NO: 9) + E (SEQ ID NO: 6)
FHLSTRDGEPLMIVAKHERGRPLLFKTTEGINKCTLIAMDLGEMCEDTVTYKCPLLVNT
EPEDIDCWCNLTSTWVMYGTCTQSGERRREKRSVALTPHSGMGLETRAETWMSSEGA
WKHAQRVESWILRNPGFALLAGFMAYMIGQTGIQRTVFFVLMMLVAPSYGMRCVGV
GNRDFVEGVSGGAWVDLVLEHGGCVTTMAQGKPTLDFELTKTTAKEVALLRTYC
IEASISNITTATRCPTQGEPYLKEEQDQQYICRRDVVDRGWGNGCGLFGKGGVVT
CAKFSCSGKITGNLVQIENLEYTVVVTVHNGDTHAVGNDTSNHGVTATITPRSPSV
EVKLPDYGELTLDCEPRSGIDFNEMILMKMKKKTWLVHKQWFLDLPLPWTAGAD
TSEVHWNYKERMVTFKVPHAKRQDVTVLGSQEGAMHSALAGATEVDSGDGNHM
FAGHLKCKVRMEKLRIKGMSYSMCTGKFKVVKEIAETQHGTIVIRVQYEGDGSPC
KIPFEIMDLEKRHVLGRLITVNPIVTEKDSPVNIEAEPPFGDSYIIIGVDPGQLKLNW
FKKGSSIGKMFESTYRGAKRMAILGETAWDFGSVGGLFTSLGKAVHQVFGSVYTT
MFGGVSWMIRILIGFLVLWIGTNSRNTSMAMTCIAVGGITLFLGFTVQA
DENV1 wt prM (SEQ ID NO: 8) + E (SEQ ID NO: 11) (GenBank ® U88535.1)
FHLTTRGGEPHMIVSKQERGKSLLFKTSAGVNMCTLIAMDLGELCEDTMTYKCPRITE
TEPDDVDCWCNATETWVTYGTCSQTGEHRRDKRSVALAPHVGLGLETRTETWMSSEG
AWKQIQKVETWALRHPGFTVIALFLAHAIGTSITQKGIIFILLMLVTPSMAMRCVGIGN
RDFVEGLSGATWVDVVLEHGSCVTTMAKDKPTLDIELLKTEVTNPAVLRKLCIEA
KISNTTTDSRCPTQGEATLVEEQDTNFVCRRTFVDRGWGNGCGLFGKGSLITCAK
FKCVTKLEGKIVQYENLKYSVIVTVHTGDQHQVGNETTEHGTTATITPQAPTSEIQ
LTDYGALTLDCSPRTGLDFNEMVLLTMEKKSWLVHKQWFLDLPLPWTSGASTSQ
ETWNRQDLLVTFKTAHAKKQEVVVLGSQEGAMHTALTGATEIQTSGTTTIFAGHL
KCRLKMDKLTLKGMSYVMCTGSFKLEKEVAETQHGTVLVQVKYEGTDAPCKIPF
SSQDEKGVTQNGRLITANPIVTDKEKPVNIEAEPPFGESYIVVGAGEKALKLSWFK
KGSSIGKMFEATARGARRMAILGDTAWDFGSIGGVFTSVGKLIHQIFGTAYGVLFS
GVSWTMKIGIGILLTWLGLNSRSTSLSMTCIAVGMVTLYLGVMVQA
DENV3 wt prM (SEQ ID NO: 12) + E (SEQ ID NO: 13) (GenBan k® JQ411814.1)
FHLTSRDGEPRMIVGKNERGKSLLFKTASGINMCTLIAMDLGEMCDDTVTYKCPHITE
VEPEDIDCWCNLTSTWVTYGTCNQAGEHRRDKRSVALAPHVGMGLDTRTQTWMSAEG
AWRQVEKVETWALRHPGFTILALFLAHYIGTSLTQKVVIFILLMLVTPSMTMRCVGIG
NRDFVEGLSGATWVDVVLEHGGCVTTMAKNKPTLDIELQKTEATQLATLRKLCIE
GKITNITTDSRCPTQGEAVLPEEQDQNYVCKHTYVDRGWGNGCGLFGKGSLVTC
AKFQCLEPIEGKVVQYENLKYTVIITVHTGDQHQVGNETQGVTAEITPQASTTEAI
LPEYGTLGLECSPRTGLDFNEMILLTMKNKAWMVHRQWFFDLPLPWTSGATTET
PTWNRKELLVTFKNAHAKKQEVVVLGSQEGAMHTALTGATEIQNSGGTSIFAGHL
KCRLKMDKLELKGMSYAMCTNTFVLKKEVSETQHGTILIKVEYKGEDAPCKIPFS
TEDGQGKAHNGRLITANPVVTKKEEPVNIEAEPPFGESNIVIGIGDNALKINWYKK
GSSIGKMFEATARGARRMAILGDTAWDFGSVGGVLNSLGKMVHQIFGSAYTALFS
GVSWVMKIGIGVLLTWIGLNSKNTSMSFSCIAIGIITLYLGAVVQA
DENV4wt prM (SEQ ID NO: 9) + E (SEQ ID NO: 14) (GenBank ® KJ160504.1)
FHLSTRDGEPLMIVAKHERGRPLLFKTTEGINKCTLIAMDLGEMCEDTVTYKCPLLVNT
EPEDIDCWCNLTSTWVMYGTCTQSGERRREKRSVALTPHSGMGLETRAETWMSSEGA
WKHAQRVESWILRNPGFALLAGFMAYMIGQTGIQRTVFFVLMMLVAPSYGMRCVGV
GNRDFVEGVSGGAWVDLVLEHGGCVTTMAQGKPTLDFELTKTTAKEVALLRTYC
IEASISNITTATRCPTQGEPYLKEEQDQQYICRRDVVDRGWGNGCGLFGKGGVVT
CAKFSCSGKITGNLVQIENLEYTVVVTVHNGDTHAVGNDTSNHGVTATITPRSPSV
EVKLPDYGELTLDCEPRSGIDFNEMILMKMKKKTWLVHKQWFLDLPLPWTAGAD
TSEVHWNYKERMVTFKVPHAKRQDVTVLGSQEGAMHSALAGATEVDSGDGNHM
FAGHLKCKVRMEKLRIKGMSYTMCSGKFSIDKEMAETQHGTTVVKVKYEGAGAP
CKVPIEIRDVNKEKVVGRVISSTPLAENTNSVTNIELEPPFGDSYIVIGVGNSALTLH
WFRKGSSIGKMFESTYRGAKRMAILGETAWDFGSVGGLFTSLGKAVHQVFGSVY
TTMFGGVSWMIRILIGFLVLWIGTNSRNTSMAMTCIAVGGITLFLGFTVQA
DENV2wt prM (SEQ ID NO: 15) + E (SEQ ID NO: 16) (GenBank ® GU289914.1)
FHLTTRNGEPHMIVSRQEKGKSLLFKTEDGVNMCTLMAMDLGELCEDTITYNCPLLRQ
NEPEDIDCWCNSTSTWVTYGTCTTTGEHRREKRSVALVPHVGMGLETRTETWMSSEG
AWKHAQRIETWILRHPGFTIMAAILAYTIGTTHFQRALIFILLTAVAPSMTMRCIGISNR
DFVEGVSGGSWVDIVLEHGSCVTTMAKNKPTLDFELIKTEAKQPATLRKYCIEAK
LTNTTTESRCPTQGEPSLNEEQDKRFVCKHSMVDRGWGNGCGLFGKGGIVTCAM
FTCKKNMEGKVVQPENLEYTIVVTPHSGEEHAVGNDTGKHGKEIKVTPQSSITEAE
LTGYGTVTMECSPRTGLDFNEMVLLQMENKAWLVHRQWFLDLPLPWLPGADTQ
GSNWIQKETLVTFKNPHAKKQDVVVLGSQEGAMHTALTGATEIQMSSGNLLFTG
HLKCRLRMDKLQLKGMSYSMCTGKFKVVKEIAETQHGTIVIRVQYEGDGSPCKIP
FEIMDLEKRHVLGRLITVNPIVTEKDSPVNIEAEPPFGDSYIIIGVDPGQLKLNWFKK
GSSIGQMFETTMRGAKRMAILGDTAWDFGSLGGVFTSIGKALHQVFGAIYGAAFS
GVSWTMKILIGVIITWIGMNSRSTSLSVSLVLVGIVTLYLGVMVQA

TABLE 1
Alignment of DENV1 wt_E(SEQ ID NO: 11), DENV1/3 EDII “small”
(SEQ ID NO: 1), DENV 1/3 EDII “large” (SEQ ID NO: 2), DENV1/3
EDIII (SEQ ID NO: 3), and DENV3 wt_E(SEQ ID NO: 13)

DENV1wt_U88535.1

| | |
|---|---|
| MRCVGIGNRDFVEGLSGATWVDVVLEHGSCVTTMAKDKPTLDIELLKTEVTNPAVLRKLC | 60 |
| DENV1/3EDIIsmall | |
| MRCVGIGNRDFVEGLSGATWVDVVLEHGSCVTTMAKDKPTLDIELLKTEVTNLATLRKLC | 60 |
| DENV1/3EDIIlarge | |
| MRCVGIGNRDFVEGLSGATWVDVVLEHGSCVTTMAKDKPTLDIELLKTEATQLATLRKLC | 60 |
| DENV1/3EDIII | |
| MRCVGIGNRDFVEGLSGATWVDVVLEHGSCVTTMAKDKPTLDIELLKTEVTNPAVLRKLC | 60 |
| DENV3wt_JQ411814.1 | |
| MRCVGIGNRDFVEGLSGATWVDVVLEHGGCVTTMAKNKPTLDIELQKTEATQLATLRKLC | 60 |
| ****************************.*******:******** ***.*: *.***** | |
| DENV1wt_U88535.1 | |
| IEAKISNTTTDSRCPTQGEATLVEEQDTNFVCRRTFVDRGWGNGCGLFGKGSLITCAKFK | 120 |
| DENV1/3EDIIsmall | |
| IEGKITNITTDSRCPTQGEAVLPEEQDQNYVCKHTYVDRGWGNGCGLFGKGSLVTCAKFQ | 120 |
| DENV1/3EDIIlarge | |
| IEGKITNITTDSRCPTQGEAVLPEEQDQNYVCKHTYVDRGWGNGCGLFGKGSLVTCAKFQ | 120 |
| DENV1/3EDIII | |
| IEAKISNTTTDSRCPTQGEATLVEEQDTNFVCRRTFVDRGWGNGCGLFGKGSLITCAKFK | 120 |
| DENV3wt_JQ411814.1 | |
| IEGKITNITTDSRCPTQGEAVLPEEQDQNYVCKHTYVDRGWGNGCGLFGKGSLVTCAKFQ | 120 |
| **.**:* ************.* **** *:**::*:*****************:*****: | |
| DENV1wt_U88535.1 | |
| CVTKLEGKIVQYENLKYSVIVTVHTGDQHQVGNETTEHGTTATITPQAPTSEIQLTDYGA | 180 |
| DENV1/3EDIIsmall | |
| CLEPIEGKVVQYENLKYSVIVTVHTGDQHQVGNETTEHGTIATITPQAPTSEIQLTDYGA | 180 |
| DENV1/3EDIIlarge | |
| CLEPIEGKVVQYENLKYSVIVTVHTGDQHQVGNETTEHGTIATITPQAPTSEIQLTDYGA | 180 |
| DENV1/3EDIII | |
| CVTKLEGKIVQYENLKYSVIVTVHTGDQHQVGNETTEHGTIATITPQAPTSEIQLTDYGA | 180 |
| DENV3wt_JQ411814.1 | |
| CLEPIEGKVVQYENLKYTVIITVHTGDQHQVGNET--QGVTAEITPQASTTEAILPEYGT | 178 |
| *: :***:********:**:************** :*. * ***** *:* * :**: | |
| DENV1wt_U88535.1 | |
| LTLDCSPRTGLDFNEMVLLTMEKKSWLVHKQWFLDLPLPWTSGASTSQETWNRQDLLVTF | 240 |
| DENV1/3EDIIsmall | |
| LTLDCSPRTGLDFNEMILLTMKNKAWMVHRQWFFDLPLPWTSGATTETPTWNRKELLVTF | 240 |
| DENV1/3EDIIlarge | |
| LTLDCSPRTGLDFNEMILLTMKNKAWMVHRQWFFDLPLPWTSGATTETPTWNRKELLVTF | 240 |
| DENV1/3EDIII | |
| LTLDCSPRTGLDFNEMVLLTMKKKSWLVHKQWFLDLPLPWTSGASTSQETWNRQDLLVTF | 240 |
| DENV3wt_JQ411814.1 | |
| LGLECSPRTGLDFNEMILLTMKNKAWMVHRQWFFDLPLPWTSGATTETPTWNRKELLVTF | 238 |
| * *:************:****::*:*:**:***:**********:*. ****::***** | |
| DENV1wt_U88535.1 | |
| KTAHAKKQEVVVLGSQEGAMHTALTGATEIQTSGTTTIFAGHLKCRLKMDKLTLKGMSYV | 300 |
| DENV1/3EDIIsmall | |
| KNAHAKKQEVVVLGSQEGAMHTALTGATEIQNSGTTTIFAGHLKCRLKMDKLTLKGMSYV | 300 |
| DENV1/3EDIIlarge | |
| KNAHAKKQEVVVLGSQEGAMHTALTGATEIQNSGGTSIFAGHLKCRLKMDKLTLKGMSYV | 300 |
| DENV1/3EDIII | |
| KTAHAKKQEVVVLGSQEGAMHTALTGATEIQTSGTTTIFAGHLKCRLKMDKLTLKGMSYA | 300 |
| DENV3wt_JQ411814.1 | |
| KNAHAKKQEVVVLGSQEGAMHTALTGATEIQNSGGTSIFAGHLKCRLKMDKLELKGMSYA | 298 |
| *.*****************************.** *:*************** ******. | |
| DENV1wt_U88535.1 | |
| MCTGSFKLEKEVAETQHGTVLVQVKYEGTDAPCKIPFSSQDEKGVTQNGRLITANPIVTD | 360 |
| DENV1/3EDIIsmall | |
| MCTGSFKLEKEVAETQHGTVLVQIKYEGTDAPCKIPFSTQDEKGVTQNGRLITANPIVTD | 360 |
| DENV1/3EDIIlarge | |
| MCTGSFKLEKEVAETQHGTVLVQIKYEGTDAPCKIPFSTQDEKGVTQNGRLITANPIVTD | 360 |
| DENV1/3EDIII | |
| MCTNTFVLKKEVSETQHGTILIKVEYKGEDAPCKIPFSTEDGQGKAHNGRLITANPVVTK | 360 |
| DENV3wt_JQ411814.1 | |
| MCTNTFVLKKEVSETQHGTILIKVEYKGEDAPCKIPFSTEDGQGKAHNGRLITANPVVTK | 358 |
| ***.:* *:***:******:*::::*:* *********::* :* ::*********:**. | |
| DENV1wt_U88535.1 | |
| KEKPVNIEAEPPFGESYIVVGAGEKALKLSWFKKGSSIGKMFEATARGARRMAILGDTAW | 420 |
| DENV1/3EDIIsmall | |
| KEKPVNIEAEPPFGESYIVIGAGEKALKLSWFKKGSSIGKMFEATARGARRMAILGDTAW | 420 |
| DENV1/3EDIIlarge | |
| KEKPVNIEAEPPFGESYIVIGAGEKALKLSWFKKGSSIGKMFEATARGARRMAILGDTAW | 420 |
| DENV1/3EDIII | |
| KEEPVNIEAEPPFGESNIVIGIGDNQHTGGRFKKGSSIGKMFEATARGARRMAILGDTAW | 420 |
| DENV3wt_JQ411814.1 | |
| KEEPVNIEAEPPFGESNIVIGIGDNALKINWYKKGSSIGKMFEATARGARRMAILGDTAW | 418 |
| **:************* **:* *:: . . :**************************** | |
| DENV1wt_U88535.1 | |
| DFGSIGGVFTSVGKLIHQIFGTAYGVLFSGVSWTMKIGIGILLTWLGLNSRSTSLSMTCI | 480 |
| DENV1/3EDIIsmall | |
| DFGSIGGVFTSVGKLVHQIFGTAYGVLFSGVSWTMKIGIGVLLTWLGLNSRSTSLSMTCI | 480 |
| DENV1/3EDIIlarge | |
| DFGSIGGVFTSVGKLVHQIFGTAYGVLFSGVSWTMKIGIGVLLTWLGLNSRSTSLSMTCI | 480 |
| DENV1/3EDIII | |
| DFGSVGGVLNSLGKMVHQIFGSAYTALFSGVSWVMKIGIGVLLTWIGLNSKNTSMSFSCI | 480 |
| DENV3wt_JQ411814.1 | |
| DFGSVGGVLNSLGKMVHQIFGSAYTALFSGVSWVMKIGIGVLLTWIGLNSKNTSMSFSCI | 478 |
| ****:***:.*:**::*****:** .*******.******:****:****:.**:*::** | |
| DENV1wt_U88535.1 AVGMVTLYLGVMVQA | 495 |
| DENV1/3EDIIsmall AVGLVTLYLGVMVQA | 495 |
| DENV1/3EDIIlarge AVGLVTLYLGVMVQA | 495 |
| DENV1/3EDIII AIGIITLYLGPYFAT | 495 |
| DENV3wt_JQ411814.1 AIGIITLYLGAVVQA | 493 |
| *:*::***** . : | |

TABLE 2
Alignments of DENV4 wt_E (SEQ ID NO: 14), DENV42 EDI
(SEQ ID NO: 4), DENV42 EDII (SEQ ID NO: 5), DENV42 EDIII
(SEQ ID NO: 6), and DENV2 wt_E (SEQ ID NO: 16)

| | |
|---|---|
| DENV4wt_KJ160504.1 | |
| MRCVGVGNRDFVEGVSGGAWVDLVLEHGGCVTTMAQGKPTLDFELTKTTAKEVALLRTYC | 60 |
| DENV42EDI | |
| MRCIGISNRDFVEGVSGGSWVDIVLEHGSCVTTMAQNKPTLDFELIKTEAKEVALLRTYC | 60 |
| DENV42EDII | |
| MRCVGVGNRDFVEGVSGGAWVDLVLEHGGCVTTMAQGKPTLDFELIKTEAKQPATLRKYC | 60 |
| DENV42EDIII | |
| MRCVGVGNRDFVEGVSGGAWVDLVLEHGGCVTTMAQGKPTLDFELTKTTAKEVALLRTYC | 60 |
| DENV2wt_GU289914.1 | |
| MRCIGISNRDFVEGVSGGSWVDIVLEHGSCVTTMAKNKPTLDFELIKTEAKQPATLRKYC | 60 |
| ***:*:.***********:***:*****.******:.******** ** **: * **.** | |
| DENV4wt_KJ160504.1 | |
| IEASISNITTATRCPTQGEPYLKEEQDQQYICRRDVVDRGWGNGCGLFGKGGVVTCAKFS | 120 |
| DENV42EDI | |
| IEASISNITTATRCPTQGEPYLKEEQDQQYICRRDVVDRGWGNGCGLFGKGGVVTCAKFS | 120 |
| DENV42EDII | |
| IEAKLTNTTTESRCPTQGEPSLNEEQDKRFVCKHSMVDRGWGNGCGLFGKGGIVTCAMFT | 120 |
| DENV42EDIII | |
| IEASISNITTATRCPTQGEPYLKEEQDQQYICRRDVVDRGWGNGCGLFGKGGVVTCAKFS | 120 |
| ***.::* ** :******** *:****::::*::.:****************:**** *: | |
| DENV2wt_GU289914.1 | |
| IEAKLTNTTTESRCPTQGEPSLNEEQDKRFVCKHSMVDRGWGNGCGLFGKGGIVTCAMFT | 120 |
| DENV4wt_KJ160504.1 | |
| CSGKITGNLVQIENLEYTVVVTVHNGDTHAVGNDTSNHGVTATITPRSPSVEVKLPDYGE | 180 |
| DENV42EDI | |
| CSGKITGNLVQPENLEYTIVVTPHSGEEHAVGNDTGKHGKEIKVTPQSSITEAELIGYGT | 180 |
| DENV42EDII | |
| CKKNMEGKVVQPENLEYTVVVTVHNGDTHAVGNDTSNHGVTATITPRSPSVEVKLPGYGE | 180 |
| DENV42EDIII | |
| CSGKITGNLVQIENLEYTVVVTVHNGDTHAVGNDTSNHGVTATITPRSPSVEVKLPDYGE | 180 |
| DENV2wt_GU289914.1 | |
| CKKNMEGKVVQPENLEYTIVVTPHSGEEHAVGNDTGKHGKEIKVTPQSSITEAELTGYGT | 180 |
| *. :: *::** ******:*** *.*: *******.:** .:**:* .*.:* .** | |
| DENV4wt_KJ160504.1 | |
| LTLDCEPRSGIDFNEMILMKMKKKTWLVHKQWFLDLPLPWTAGADTSEVHWNYKERMVTF | 240 |
| DENV42EDI | |
| VTMECSPRTGLDFNEMILMKMKKKTWLVHKQWFLDLPLPWTAGADTSEVHWNYKERMVTF | 240 |
| DENV42EDII | |
| LTLDCEPRSGLDFNEMVLLQMENKAWLVHRQWFLDLPLPWLPGADTQGSNWIQKETLVTF | 240 |
| DENV42EDIII | |
| LTLDCEPRSGIDFNEMILMKMKKKTWLVHKQWFLDLPLPWTAGADTSEVHWNYKERMVTF | 240 |
| DENV2wt_GU289914.1 | |
| VTMECSPRTGLDFNEMVLLQMENKAWLVHRQWFLDLPLPWLPGADTQGSNWIQKETLVTF | 240 |
| :*::*.**:*:*****:*::*::*:****:********** ****. :* ** :*** | |
| DENV4wt_KJ160504.1 | |
| KVPHAKRQDVTVLGSQEGAMHSALAGATEVDSGDGNHMFAGHLKCKVRMEKLRIKGMSYT | 300 |
| DENV42EDI | |
| KVPHAKRQDVTVLGSQEGAMHSALAGATEVDSGDGNHLFAGHLKCRLRMDKLQLKGMSYT | 300 |
| DENV42EDII | |
| KNPHAKKQDVVVLGSQEGAMHTALTGATEIQMSSGNLLFTGHLKCKVRMEKLRIKGMSYT | 300 |
| DENV42EDIII | |
| KVPHAKRQDVTVLGSQEGAMHSALAGATEVDSGDGNHMFAGHLKCKVRMEKLRIKGMSYS | 300 |
| DENV2wt_GU289914.1 | |
| KNPHAKKQDVVVLGSQEGAMHTALTGATEIQMSSGNLLFTGHLKCRLRMDKLQLKGMSYS | 300 |
| * ****:***.**********:**:****:: ..** :*:*****::**:**::*****: | |
| DENV4wt_KJ160504.1 | |
| MCSGKFSIDKEMAETQHGTTVVKVKYEGAGAPCKVPIEIRDVNKEKVVGRVISSTPLAEN | 360 |
| DENV42EDI | |
| MCSGKFKIDKEMAETQHGTTVVKVKYEGAGAPCKVPIEIRDVNKEKVVGRVISSTPLAEN | 360 |
| DENV42EDII | |
| MCSGKFSIDKEMAETQHGTTVVKVKYEGAGAPCKVPIEIRDVNKEKVVGRVISSTPLAEN | 360 |
| DENV42EDIII | |
| MCTGKFKVVKEIAETQHGTIVIRVQYEGDGSPCKIPFEIMDLEKRHVLGRLITVNPIVTE | 360 |
| DENV2wt_GU289914.1 | |
| MCTGKFKVVKEIAETQHGTIVIRVQYEGDGSPCKIPFEIMDLEKRHVLGRLITVNPIVTE | 360 |
| **:***.: **:******* *::*:*** *:***:*:** *::*.:*:**:*: .*:. : | |
| DENV4wt_KJ160504.1 | |
| TNSVTNIELEPPFGDSYIVIGVGNSALTLHWFRKGSSIGKMFESTYRGAKRMAILGETAW | 420 |
| DENV42EDI | |
| TNSVTNIELEPPFGDSYIVIGVGNSALTLHWFRKGSSIGKMFESTYRGAKRMAILGETAW | 420 |
| DENV42EDII | |
| TNSVTNIELEPPFGDSYIVIGVGNSALTLHWFRKGSSIGKMFESTYRGAKRMAILGETAW | 420 |
| DENV42EDIII | |
| KDSPVNIEAEPPFGDSYIIIGVDPGQLKLNWFKKGSSIGKMFESTYRGAKRMAILGETAW | 420 |
| DENV2wt_GU289914.1 | |
| KDSPVNIEAEPPFGDSYIIIGVDPGQLKLNWFKKGSSIGQMFETTMRGAKRMAILGDTAW | 420 |
| .:* .*** *********:***. . *.*:**:******:***:* **********:*** | |
| DENV4wt_KJ160504.1 | |
| DFGSVGGLFTSLGKAVHQVFGSVYTTMFGGVSWMIRILIGFLVLWIGTNSRNTSMAMTCI | 480 |
| DENV42EDI | |
| DFGSVGGLFTSLGKAVHQVFGSVYTTMFGGVSWMIRILIGFLVLWIGTNSRNTSMAMTCI | 480 |
| DENV42EDII | |
| DFGSVGGLFTSLGKAVHQVFGSVYTTMFGGVSWMIRILIGFLVLWIGTNSRNTSMAMTCI | 480 |
| DENV42EDIII | |
| DFGSVGGLFTSLGKAVHQVFGSVYTTMFGGVSWMIRILIGFLVLWIGTNSRNTSMAMTCI | 480 |
| DENV2wt_GU289914.1 | |
| DFGSLGGVFTSIGKALHQVFGAIYGAAFSGVSWTMKILIGVIITWIGMNSRSTSLSVSLV | 480 |
| ****:**:***:***:*****::* : *.**** ::****.:: *** ***.**:::: : | |
| DENV4wt_KJ160504.1 AVGGITLFLGFTVQA | 495 |
| DENV42EDI AVGGITLFLGFTVQA | 495 |
| DENV42EDII AVGGITLFLGFTVQA | 495 |
| DENV42EDIII AVGGITLFLGFTVQA | 495 |
| DENV2wt_GU289914.1 LVGIVTLYLGVMVQA | 495 |
| ** :**:**. *** | |

TABLE 3
Substitution positions in the DENV1/3 chimeras as compared to DENV1 wildtype
E and DENV3 wildtype E glycoprotein, wherein the numbering corresponds to
the amino acid sequence of wildtype DENV1 E glycoprotein (SEQ ID NO: 11).

| | 29 | 37 | 46 | 50 | 52 | 53 | 55 | 63 | 66 | 68 | 81 | 83 |
|---|---|---|---|---|---|---|---|---|---|---|---|---|
| DENV1 | S | D | L | V | N | P | V | A | S | T | T | V |
| DENV1/3 | S | D | L | V | N | L | T | G | T | I | V | P |
| EDII small | | | | | | | | | | | | |
| DENV1/3 | S | D | L | A | Q | L | T | G | T | I | V | P |
| EDII large | | | | | | | | | | | | |
| DENV1/3 | S | D | L | V | N | P | V | A | S | T | T | V |
| EDIII | | | | | | | | | | | | |
| DENV3 | G | N | Q | A | Q | L | T | G | T | I | V | P |

| | 88 | 90 | 93 | 94 | 96 | 114 | 120 | 122 | 123 | 124 | 125 | 129 |
|---|---|---|---|---|---|---|---|---|---|---|---|---|
| DENV1 | T | F | R | R | F | I | K | V | T | K | L | I |
| DENV1/3 | Q | Y | K | H | Y | V | Q | L | E | P | I | V |
| EDII small | | | | | | | | | | | | |
| DENV1/3 | Q | Y | K | H | Y | V | Q | L | E | P | I | V |
| EDII large | | | | | | | | | | | | |
| DENV1/3 | T | F | R | R | F | I | K | V | T | K | L | I |
| EDIII | | | | | | | | | | | | |
| DENV3 | Q | Y | K | H | Y | V | Q | L | E | P | I | V |

| | 138 | 141 | 156 | 157 | 158 | 160 | 161 | 163 | 169 | 171 | 173 | 174 |
|---|---|---|---|---|---|---|---|---|---|---|---|---|
| DENV1 | S | V | T | E | H | T | T | T | P | S | I | Q |
| DENV1/3 | S | V | T | E | H | T | I | T | P | S | I | Q |
| EDII small | | | | | | | | | | | | |
| DENV1/3 | S | V | T | E | H | T | I | T | P | S | I | Q |
| EDII large | | | | | | | | | | | | |
| DENV1/3 | S | V | T | E | H | T | I | T | P | S | I | Q |
| EDIII | | | | | | | | | | | | |
| DENV3 | T | I | — | — | Q | V | T | E | S | T | A | I |
| | 176 | 177 | 180 | 182 | 184 | 197 | 202 | 203 | 205 | 207 | 210 | 214 |
| DENV1 | T | D | A | T | D | V | K | K | S | L | K | L |
| DENV1/3 | T | D | A | T | D | I | K | N | A | M | R | F |
| EDII small | | | | | | | | | | | | |
| DENV1/3 | T | D | A | T | D | I | K | N | A | M | R | F |
| EDII large | | | | | | | | | | | | |
| DENV1/3 | T | D | A | T | D | V | K | K | S | L | K | L |
| EDIII | | | | | | | | | | | | |
| DENV3 | P | E | T | G | E | I | K | N | A | M | R | F |

| | 225 | 22 | 228 | 229 | 234 | 235 | 242 | 272 | 275 | 277 | 293 | 300 |
|---|---|---|---|---|---|---|---|---|---|---|---|---|
| DENV1 | S | S | Q | E | Q | D | T | T | T | T | T | V |
| DENV1/3 | T | E | T | P | K | E | N | N | T | T | T | V |
| EDII small | | | | | | | | | | | | |
| DENV1/3 | T | E | T | P | K | E | N | N | G | S | T | V |
| EDII large | | | | | | | | | | | | |
| DENV1/3 | S | S | Q | E | Q | D | T | T | T | T | T | A |
| EDIII | | | | | | | | | | | | |
| DENV3 | T | E | T | P | K | E | N | N | G | S | E | A |

| | 304 | 305 | 307 | 309 | 313 | 320 | 322 | 323 | 324 | 325 | 327 | 329 |
|---|---|---|---|---|---|---|---|---|---|---|---|---|
| DENV1 | G | S | K | E | A | V | V | Q | V | K | E | T |
| DENV1/3 | G | S | K | E | A | V | V | Q | I | K | E | T |
| EDII small | | | | | | | | | | | | |
| DENV1/3 | G | S | K | E | A | V | V | Q | I | K | E | T |
| EDII large | | | | | | | | | | | | |
| DENV1/3 | N | T | V | K | S | I | I | K | V | E | K | E |
| EDIII | | | | | | | | | | | | |
| DENV3 | N | T | V | K | S | I | I | K | V | E | K | E |
| | 339 | 340 | 342 | 343 | 345 | 346 | 347 | 357 | 360 | 363 | 377 | 380 |
| DENV1 | S | Q | E | K | V | T | Q | I | D | K | Y | V |
| DENV1/3 | T | Q | E | K | V | T | Q | I | D | K | Y | I |
| EDII small | | | | | | | | | | | | |
| DENV1/3 | T | Q | E | K | V | T | Q | I | D | K | Y | I |
| EDII large | | | | | | | | | | | | |
| DENV1/3 | T | E | G | Q | K | A | H | V | K | E | N | I |
| EDIII | | | | | | | | | | | | |
| DENV3 | T | E | G | Q | K | A | H | V | K | E | N | I |
| | 382 | 384 | 385 | 386 | 387 | 388 | 389 | 390 | 391 | 392 | 425 | 429 |
| DENV1 | A | E | K | A | L | K | L | S | W | F | I | F |
| DENV1/3 | A | E | K | A | L | K | L | S | W | F | I | F |
| EDII small | | | | | | | | | | | | |
| DENV1/3 | A | E | K | A | L | K | L | S | W | F | I | F |
| EDII large | | | | | | | | | | | | |
| DENV1/3 | I | D | N | Q | H | T | G | G | R | F | V | L |
| EDIII | | | | | | | | | | | | |
| DENV3 | I | D | N | A | L | K | I | N | W | Y | V | L |
| | 430 | 432 | 435 | 436 | 442 | 445 | 446 | 454 | 461 | 466 | 471 | 472 |
| DENV1 | T | V | L | I | T | G | V | T | I | L | R | S |
| DENV1/3 | T | V | L | V | T | G | V | T | V | L | R | S |
| EDII small | | | | | | | | | | | | |
| DENV1/3 | T | V | L | V | T | G | V | T | V | L | R | S |
| EDII large | | | | | | | | | | | | |
| DENV1/3 | N | L | M | V | S | T | A | V | V | I | K | N |
| EDIII | | | | | | | | | | | | |
| DENV3 | N | L | M | V | S | T | A | V | V | I | K | N |
| | 475 | 477 | 478 | 482 | 484 | 485 | 491 | 492 | 493 | 494 | 495 | 496 |
| DENV1 | L | M | T | V | M | V | V | M | V | Q | A | D |
| DENV1/3 | L | M | T | V | L | V | V | M | V | Q | A | D |
| EDII small | | | | | | | | | | | | |
| DENV1/3 | L | M | T | V | L | V | V | M | V | Q | A | D |
| EDII large | | | | | | | | | | | | |
| DENV1/3 | M | F | S | I | I | I | P | Y | F | A | T | Q |
| EDIII | | | | | | | | | | | | |
| DENV3 | M | F | S | I | I | I | A | V | V | Q | A | D |

| | 497 | 498 |
|---|---|---|
| DENV1 | S | G |
| DENV1/3 | S | G |
| EDII small | | |
| DENV1/3 | S | G |
| EDII large | | |
| DENV1/3 | L | Q |
| EDIII | | |
| DENV3 | M | G |

TABLE 4
Substitution positions in the DENV2/4 chimeras as compared to DENV4 wildtype
E and DENV2 wildtype E glycoprotein, wherein the numbering corresponds to
the amino acid sequence wildtype DENV4 E glycoprotein (SEQ ID NO: 14).

| | 4 | 6 | 7 | 19 | 23 | 29 | 36 | 37 | 46 | 49 | 52 | 53 |
|---|---|---|---|---|---|---|---|---|---|---|---|---|
| DENV4 | V | V | G | A | L | G | Q | G | T | T | E | V |
| DENV4/2 | I | I | S | S | I | S | Q | N | I | E | E | V |
| EDI | | | | | | | | | | | | |
| DENV4/2 | V | V | G | A | L | G | Q | G | I | E | Q | P |
| EDII | | | | | | | | | | | | |
| DENV4/2 | V | V | G | A | L | G | Q | G | T | T | E | V |
| EDIII | | | | | | | | | | | | |
| DENV2 | I | I | S | S | I | S | K | N | I | E | Q | P |
| | 55 | 58 | 64 | 65 | 66 | 68 | 71 | 72 | 81 | 83 | 88 | 89 |
| DENV4 | L | T | S | I | S | I | A | T | Y | K | Q | Q |
| DENV4/2 | L | T | S | I | S | I | A | T | Y | K | Q | Q |
| EDI | | | | | | | | | | | | |
| DENV4/2 | T | K | K | L | T | T | E | S | S | N | K | R |
| EDII | | | | | | | | | | | | |
| DENV4/2 | L | T | S | I | S | I | A | T | Y | K | Q | Q |
| EDIII | | | | | | | | | | | | |
| DENV2 | T | K | K | L | T | T | E | S | S | N | K | R |

| | 90 | 91 | 93 | 94 | 95 | 96 | 113 | 118 | 120 | 122 | 123 | 124 |
|---|---|---|---|---|---|---|---|---|---|---|---|---|
| DENV4 | Y | I | R | R | D | V | V | K | S | S | G | K |
| DENV4/2 | Y | I | R | R | D | V | V | K | S | S | G | K |
| EDI | | | | | | | | | | | | |
| DENV4/2 | F | V | K | H | S | M | I | M | T | K | K | N |
| EDII | | | | | | | | | | | | |
| DENV4/2 | Y | I | R | R | D | V | V | K | S | S | G | K |
| EDIII | | | | | | | | | | | | |
| DENV2 | F | V | K | H | S | M | I | M | T | K | K | N |

| | 125 | 126 | 128 | 129 | 132 | 139 | 143 | 145 | 147 | 148 | 156 | 157 |
|---|---|---|---|---|---|---|---|---|---|---|---|---|
| DENV4 | I | T | N | L | I | V | V | N | D | T | S | N |
| DENV4/2 | I | T | N | L | P | I | P | S | E | E | G | K |
| EDI | | | | | | | | | | | | |
| DENV4/2 | M | E | K | V | P | V | V | N | D | T | S | N |
| EDII | | | | | | | | | | | | |
| DENV4/2 | I | T | N | L | I | V | V | N | D | T | S | N |
| EDIII | | | | | | | | | | | | |
| DENV2 | M | E | K | V | P | I | P | S | E | E | G | K |
| | 160 | 161 | 162 | 163 | 164 | 167 | 169 | 170 | 171 | 173 | 174 | 176 |
| DENV4 | V | T | A | T | I | R | P | S | V | V | K | P |
| DENV4/2 | K | E | I | K | V | Q | S | I | T | A | E | T |
| EDI | | | | | | | | | | | | |
| DENV4/2 | V | T | A | T | I | R | P | S | V | V | K | P |
| EDII | | | | | | | | | | | | |
| DENV4/2 | V | T | A | T | I | R | P | S | V | V | K | P |
| EDIII | | | | | | | | | | | | |
| DENV2 | K | E | I | K | V | Q | S | I | T | A | E | T |
| | 177 | 180 | 181 | 183 | 184 | 186 | 189 | 191 | 197 | 199 | 200 | 202 |
| DENV4 | D | E | L | L | D | E | S | I | I | M | K | K |
| DENV4/2 | G | T | V | M | E | S | T | L | I | M | K | K |
| EDI | | | | | | | | | | | | |
| DENV4/2 | G | E | L | L | D | E | S | L | V | L | Q | E |
| EDII | | | | | | | | | | | | |
| DENV4/2 | D | E | L | L | D | E | S | I | I | M | K | K |
| EDIII | | | | | | | | | | | | |
| DENV2 | G | T | V | M | E | S | T | L | V | L | Q | E |
| | 203 | 205 | 210 | 221 | 222 | 227 | 228 | 229 | 230 | 232 | 233 | 236 |
| DENV4 | K | T | K | T | A | S | E | V | H | N | Y | R |
| DENV4/2 | K | T | K | T | A | S | E | V | H | N | Y | R |
| EDI | | | | | | | | | | | | |
| DENV4/2 | N | A | R | L | P | Q | G | S | N | I | Q | T |
| EDII | | | | | | | | | | | | |
| DENV4/2 | K | T | K | T | A | S | E | V | H | N | Y | R |
| EDIII | | | | | | | | | | | | |
| DENV2 | N | A | R | L | P | Q | G | S | N | I | Q | T |
| | 237 | 242 | 247 | 251 | 262 | 265 | 270 | 271 | 272 | 273 | 274 | 277 |
| DENV4 | M | V | R | T | S | A | V | D | S | G | D | H |
| DENV4/2 | M | V | R | T | S | A | V | D | S | G | D | H |
| EDI | | | | | | | | | | | | |
| DENV4/2 | L | N | K | V | T | T | I | Q | M | S | S | L |
| EDII | | | | | | | | | | | | |
| DENV4/2 | M | V | R | T | S | A | V | D | S | G | D | H |
| EDIII | | | | | | | | | | | | |
| DENV2 | L | N | K | V | T | T | I | Q | M | S | S | L |
| | 278 | 280 | 286 | 287 | 290 | 293 | 294 | 300 | 303 | 307 | 308 | 309 |
| DENV4 | M | A | K | V | E | R | I | T | S | S | I | D |
| DENV4/2 | L | A | R | L | D | Q | L | T | S | K | I | D |
| EDI | | | | | | | | | | | | |
| DENV4/2 | L | T | K | V | E | R | I | T | S | S | I | D |
| EDII | | | | | | | | | | | | |
| DENV4/2 | M | A | K | V | E | R | I | S | T | K | V | V |
| EDIII | | | | | | | | | | | | |
| DENV2 | L | T | R | L | D | Q | L | S | T | K | V | V |
| | 312 | 320 | 322 | 323 | 325 | 329 | 331 | 335 | 337 | 340 | 342 | 343 |
| DENV4 | M | T | V | K | K | A | A | V | I | R | V | N |
| DENV4/2 | M | T | V | K | K | A | A | V | I | R | v | N |
| EDI | | | | | | | | | | | | |
| DENV4/2 | M | T | V | K | K | A | A | V | I | R | V | N |
| EDII | | | | | | | | | | | | |
| DENV4/2 | I | I | I | R | Q | D | S | I | F | M | L | E |
| EDIII | | | | | | | | | | | | |
| DENV2 | I | I | I | R | Q | D | S | I | F | M | L | E |
| | 345 | 346 | 348 | 351 | 353 | 354 | 355 | 357 | 358 | 359 | 360 | 361 |
| DENV4 | E | K | V | V | S | S | T | L | A | E | N | T |
| DENV4/2 | E | K | V | V | S | S | T | L | A | E | N | T |
| EDI | | | | | | | | | | | | |
| DENV4/2 | E | K | V | V | S | S | T | l | A | E | N | T |
| EDII | | | | | | | | | | | | |
| DENV4/2 | R | H | L | L | T | V | N | I | V | T | E | K |
| EDIII | | | | | | | | | | | | |
| DENV2 | R | H | L | L | T | V | N | I | V | T | E | K |
| | 362 | 364 | 365 | 369 | 379 | 383 | 384 | 385 | 386 | 388 | 390 | 393 |
| DENV4 | N | V | T | L | V | G | N | S | A | T | H | R |
| DENV4/2 | N | V | T | L | V | G | N | S | A | T | H | R |
| EDI | | | | | | | | | | | | |
| DENV4/2 | N | V | T | L | V | G | N | S | A | T | H | R |
| EDII | | | | | | | | | | | | |
| DENV4/2 | D | P | V | A | I | D | P | G | Q | K | N | K |
| EDIII | | | | | | | | | | | | |
| DENV2 | D | P | V | A | I | D | P | G | Q | K | N | K |
| | 400 | 404 | 406 | 417 | 425 | 428 | 432 | 436 | 442 | 443 | 445 | 446 |
| DENV4 | K | S | Y | E | V | L | L | V | S | V | T | T |
| DENV4/2 | K | S | Y | E | V | L | L | V | S | V | T | T |
| EDI | | | | | | | | | | | | |
| DENV4/2 | K | S | Y | E | V | L | L | V | S | V | T | T |
| EDII | | | | | | | | | | | | |
| DENV4/2 | K | S | Y | E | V | L | L | V | S | V | T | T |
| EDIII | | | | | | | | | | | | |
| DENV2 | Q | T | M | D | L | V | I | L | A | I | G | A |
| | 447 | 449 | 454 | 455 | 456 | 461 | 462 | 463 | 464 | 468 | 472 | 475 |
| DENV4 | M | G | M | I | R | F | L | V | L | T | N | M |
| DENV4/2 | M | G | M | I | R | F | L | V | L | T | N | M |
| EDI | | | | | | | | | | | | |
| DENV4/2 | M | G | M | I | R | F | L | V | L | T | N | M |
| EDII | | | | | | | | | | | | |
| DENV4/2 | M | G | M | I | R | F | I | V | L | T | N | M |
| EDIII | | | | | | | | | | | | |
| DENV2 | A | S | T | M | K | V | I | I | T | M | S | L |

| | 476 | 477 | 478 | 479 | 480 | 481 | 484 | 485 | 488 | 491 | 492 |
|---|---|---|---|---|---|---|---|---|---|---|---|
| DENV4 | A | M | T | C | I | A | G | I | F | F | T |
| DENV4/2 | A | M | T | C | I | A | G | I | F | F | T |
| EDI | | | | | | | | | | | |
| DENV4/2 | A | M | T | C | I | A | G | I | F | F | T |
| EDII | | | | | | | | | | | |
| DENV4/2 | A | M | T | C | I | A | G | I | F | F | T |
| EDIII | | | | | | | | | | | |
| DENV2 | S | V | S | L | V | L | I | V | Y | V | M |

TABLE 5
Additional EDII and EDIII Constructs. E-glycoprotein DENV3 encoded changes
into DENV1 are represented for DENV1/3 EDII and EDIII chimeras.

| | | HINGE | | | DOMAIN 2 | | | | | | | | | | |
|---|---|---|---|---|---|---|---|---|---|---|---|---|---|---|---|
| E-Protein | DOMAIN 1 | 46 | 50 | 52 | 53 | 55 | 63 | 66 | 68 | 81 | 83 | 88 | 90 | 93 | 94 |
| DENV3 | | Q | A | Q | L | T | G | T | I | V | P | Q | Y | K | H |
| DENV1-3 | | L | V | N | L | T | G | T | I | V | P | Q | Y | K | H |
| EDII A | | | | | | | | | | | | | | | |
| DENV1-3 | | L | V | N | P | V | A | S | T | T | V | T | F | R | R |
| EDIIIb | | | | | | | | | | | | | | | |
| DENV1 | | L | V | N | P | V | A | S | T | T | V | T | F | R | R |

| | DOMAIN 2 | | | | | | | HINGE | | | | | | DOMAIN 2 | |
|---|---|---|---|---|---|---|---|---|---|---|---|---|---|---|---|
| E-Protein | 96 | 114 | 120 | 122 | 123 | 124 | 125 | 129 | 197 | 203 | 205 | 207 | 210 | 214 | 225 |
| DENV3 | Y | V | Q | L | E | P | I | V | I | N | A | M | R | F | T |
| DENV1-3 | Y | V | Q | L | E | P | I | V | I | N | A | M | R | F | T |
| EDII | | | | | | | | | | | | | | | |
| DENV1-3 | F | I | K | V | T | K | L | I | V | K | S | L | K | L | S |
| EDIII | | | | | | | | | | | | | | | |
| DENV1-3 | F | I | K | V | T | K | L | I | V | K | S | L | K | L | S |
| EDIII | | | | | | | | | | | | | | | |

| DOMAIN 2 | | | | | | | HINGE | | | DOMAIN 3 | | | | | |
|---|---|---|---|---|---|---|---|---|---|---|---|---|---|---|---|
| E-Protein | 227 | 228 | 229 | 234 | 235 | 242 | 272 | 275 | 277 | 299-340 | 341 | 342 | 343 | 344 | 345 |
| DENV3 | E | T | P | K | E | N | N | G | S | | T | E | D | G | Q |
| DENV1-3 | E | T | P | K | E | N | N | T | T | | S | Q | D | E | K |
| EDII | | | | | | | | | | | | | | | |
| DENV1-3 | S | Q | E | Q | D | T | T | T | T | | T | E | D | G | Q |
| EDIII | | | | | | | | | | | | | | | |
| DENV1 | S | Q | E | Q | D | T | T | T | T | | S | Q | D | E | K |

| DOMAIN 3 | | | | | | |
|---|---|---|---|---|---|---|
| E-Protein | 347 | 348 | 349 | 350-391 | 392 | 393-end |
| DENV3 | K | A | H | | Y | |
| DENV1-3 | V | T | Q | | F | |
| EDII | | | | | | |
| DENV1-3 | K | A | H | | F | |
| EDIII | | | | | | |
| DENV1 | V | T | Q | | F | |

TABLE 6
DENV1-3 EDII Chimeric prM.

| | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| DENV3 | F | H | L | T | S | R | D | G | E | P | R | M | I | V | G | K | N | E | R | G |
| DENV1 | F | H | L | T | T | R | G | G | E | P | H | M | I | V | S | K | Q | E | R | G |
| | 21 | 22 | 23 | 24 | 25 | 26 | 27 | 28 | 29 | 30 | 31 | 32 | 33 | 34 | 35 | 36 | 37 | 38 | 39 | 40 |
| DENV3 | K | S | L | L | F | K | T | A | S | G | I | N | M | C | T | L | I | A | M | D |
| DENV1 | K | S | L | L | F | K | T | S | A | G | V | N | M | C | T | L | I | A | M | D |
| | 41 | 42 | 43 | 44 | 45 | 46 | 47 | 48 | 49 | 50 | 51 | 52 | 53 | 54 | 55 | 56 | 57 | 58 | 59 | 60 |
| DENV3 | L | G | E | M | C | D | D | T | V | T | Y | K | C | P | H | I | T | E | V | E |
| DENV1 | L | G | E | L | C | E | D | T | M | T | Y | K | C | P | R | I | T | E | T | E |
| | 61 | 62 | 63 | 64 | 65 | 66 | 67 | 68 | 69 | 70 | 71 | 72 | 73 | 74 | 75 | 76 | 77 | 78 | 79 | 80 |
| DENV3 | P | E | D | I | D | C | W | C | N | L | T | S | T | W | V | T | Y | G | T | C |
| DENV1 | P | D | D | V | D | C | W | C | N | A | T | E | T | W | V | T | Y | G | T | C |
| | 81 | 82 | 83 | 84 | 85 | 86 | 87 | 88 | 89 | 90 | 91 | 92 | 93 | 94 | 95 | 96 | 97 | 98 | 99 | 100 |
| DENV3 | N | Q | A | G | E | H | R | R | D | K | R | S | V | A | L | A | P | H | V | G |
| DENV1 | S | Q | T | G | E | H | R | R | D | K | R | S | V | A | L | A | P | H | V | G |

| | 101 | 102 | 103 | 104 | 105 | 106 | 107 | 108 | 109 | 110 |
|---|---|---|---|---|---|---|---|---|---|---|
| DENV3 | M | G | L | D | T | R | T | Q | T | W |
| DENV1 | L | G | L | E | T | R | T | E | T | W |

| | 111 | 112 | 113 | 114 | 115 | 116 | 117 | 118 | 119 | 120 |
|---|---|---|---|---|---|---|---|---|---|---|
| DENV3 | M | S | A | E | G | A | W | R | Q | V |
| DENV1 | M | S | S | E | G | A | W | K | Q | I |

TABLE 7
DENV1-3 EDII Constructs A-D.

| | | | HINGE | | | DOMAIN 2 | | | | | | | | |
|---|---|---|---|---|---|---|---|---|---|---|---|---|---|---|
| | E-Protein | DOMAIN 1 | 46 | 50 | 52 | 53 | 55 | 63 | 66 | 68 | 81 | 83 | 88 | 90 |
| | DENV3 | | Q | A | Q | L | T | G | T | I | V | P | Q | Y |
| A | DV1 hinge | | L | V | N | L | T | G | T | I | V | P | Q | Y |
| | small | | | | | | | | | | | | | |
| B | DV1 hinge | | L | V | N | P | V | G | T | I | V | P | Q | Y |
| | large | | | | | | | | | | | | | |
| C | DV3 hinge | | L | A | Q | L | T | G | T | I | V | P | Q | Y |
| | no 46 | | | | | | | | | | | | | |
| D | DV3 46 | | Q | A | Q | L | T | G | T | I | V | P | Q | Y |
| | DENV1 | | L | V | N | P | V | A | S | T | T | V | T | F |

| | | DOMAIN 2 | | | | | | | | | HINGE | | | | | |
|---|---|---|---|---|---|---|---|---|---|---|---|---|---|---|---|---|
| | E-Protein | 93 | 94 | 96 | 114 | 120 | 122 | 123 | 124 | 125 | 129 | 197 | 203 | 205 | 207 | 210 |
| | DENV3 | K | H | Y | V | Q | L | E | P | I | V | I | N | A | M | R |
| A | DV1 hinge | K | H | Y | V | Q | L | E | P | I | V | I | N | A | M | R |
| | small | | | | | | | | | | | | | | | |
| B | DV1 hinge | K | H | Y | V | Q | L | E | P | I | I | V | K | S | L | K |
| | large | | | | | | | | | | | | | | | |
| C | DV3 hinge | K | H | Y | V | Q | L | E | P | I | V | I | N | A | M | R |
| | no 46 | | | | | | | | | | | | | | | |
| D | DV3 46 | K | H | Y | V | Q | L | E | P | I | V | I | N | A | M | R |
| | DENV1 | R | R | F | I | K | V | T | K | L | I | V | K | S | L | K |

| | DOMAIN 2 | | | | | | | | | HINGE | | | |
|---|---|---|---|---|---|---|---|---|---|---|---|---|---|
| | E-Protein | 214 | 225 | 227 | 228 | 229 | 234 | 235 | 242 | 272 | 275 | 277 | DOMAIN 3 |
| | DENV3 | F | T | E | T | P | K | E | N | N | G | S | |
| A | DV1 hinge | F | T | E | T | P | K | E | N | N | T | T | |
| | small | | | | | | | | | | | | |
| B | DV1 hinge | F | T | E | T | P | K | E | N | T | T | T | |
| | large | | | | | | | | | | | | |
| C | DV3 hinge | F | T | E | T | P | K | E | N | N | G | S | |
| | no 46 | | | | | | | | | | | | |
| D | DV3 46 | F | T | E | T | P | K | E | N | N | G | S | |
| | DENV1 | L | S | S | Q | E | Q | D | T | T | T | T | |

TABLE 8
DENV 1-3 EDIII constructs a-d.

| | | | | | | | | | | | | 393-end |
|---|---|---|---|---|---|---|---|---|---|---|---|---|
| Domain 1 | 299-340 | 341 | 342 | 343 | 344 | 345 | 346 | 347 | 348 | 349 | 392 | Domain 3 |

| | | | | | | | | | | | |
|---|---|---|---|---|---|---|---|---|---|---|---|
| DENV3 | T | E | D | G | Q | G | K | A | H | Y | DENV3 |
| a | T | E | D | E | K | G | K | A | H | Y | a |
| b | T | E | D | G | Q | G | K | A | H | F | b |
| c | T | E | D | E | K | G | K | T | H | Y | c |
| d | T | E | D | G | Q | G | K | A | H | Y | d |
| DENV1 | S | Q | D | E | K | G | V | T | Q | F | DENV1 |

TABLE 9
DENV3/3 C1-C4, G-IV to G-III residue changes.

| | 62 | 63 | 68 | 81 | 113 | 120 | 124 | 224 | 226 |
|---|---|---|---|---|---|---|---|---|---|
| DENV3 | E | G | I | V | L | Q | P | T | T |
| GIII | | | | | | | | | |
| Cluster 1 | G | A | I | V | L | H | L | A | V |
| Cluster 2 | G | A | T | T | S | H | L | T | T |
| Cluster 3 | E | G | T | T | S | Q | P | A | V |
| Cluster 4 | E | G | T | T | S | Q | P | T | T |
| DENV3 | G | A | T | T | S | H | L | A | V |
| GIV | | | | | | | | | |

TABLE 10

| | | EDII > | EDII > | EDII > | | EDII > | | | | EDI > |
|---|---|---|---|---|---|---|---|---|---|---|
| Structural | | EDI > | EDIII > | EDI > | EDI > | EDIII > | | EDIII > | | EDIII > |
| Target | EDII | EDIII | EDI | EDIII | EDII | EDI | EDIII | EDI | EDI | EDII |
| Country | Nicaragua | Sri Lanka | Nicaragua | Nicaragua | Nicaragua | Nicaragua | ? | ? | ? | ? |
| Time of | 14 years | 1.5 years | 18 years | <1 year | <1 year | <1 year | 180 days | 180 days | 180 days | 180 days |
| Draw | | | | | | | | | | |
| Year | 1995 | 2011 | 1998 | 2010 | 2010 | 2010 | N/A | N/A | N/A | N/A |
| Infected | | | | | | | | | | |
| Age | Adult | Adult | Adult | Child | Child | Child | Adult | Adult | Adult | Adult |
| Genotype | GIII | GIII | GIII | GIII | GIII | GIII | GII | GII | GII | GII |
| Immune | Natural | Natural | Natural | Natural | Natural | Natural | Vaccine | Vaccine | Vaccine | Vaccine |
| Status | | | | | | | | | | |
| Source | Travelers' | Travelers' | Travelers' | Eva/Nica | Eva/Nica | Eva/Nica | monoval | monoval | monoval | monoval |
| Serum | DT103 | DT133 | DT226 | 4082.7 | 4862.7 | 5862.7 | NIH2 | NIH16 | NIH47 | NIH50 |
| Sample ID | ATC A | ATC B | ATC C | NPDCS A | NPDCS B | NPDCS C | NIH A | NIH B | NIHD | NIH F |

TABLE 11

| Natural | | DENV3 | DENV3 | DENV1-3 | DENV1-3 | DENV1-3 |
|---|---|---|---|---|---|---|
| Serum | DENV1 | GII | GIII | EDI | EDII | EDIII |
| ATC A % TS | 0/<40 | 84/153 | 63/109 | 0/<40 | 93/129 | 0/<40 |
| ATC A % CR | 100/90 | 16/30 | 37/65 | 100/394 | 7/9 | 0/<40 |
| ATC B % TS | 84/147 | 59/941 | 51/3086 | 17/184 | 40/492 | 6/45 |
| ATC B % CR | 16/29 | 31/642 | 49/2879 | 83/899 | 60/746 | 94/766 |
| ATC C % TS | 0/<40 | 45/3406 | 83/7205 | 40/818 | 100/3827 | 100/1104 |
| ATC C % CR | 100/268 | 55/4221 | 17/1427 | 60/1205 | 0/<40 | 0/<40 |
| NPDCS A % TS | 0/<40 | 100/2063 | 48/949 | 39/331 | 99/1209 | 26/158 |
| NPDCS A % CR | 0/<40 | 0/<40 | 52/1022 | 61/507 | 1/18 | 74/451 |
| NPDCS B % TS | 0/<40 | 59/533 | 32/621 | 17/173 | 24/99 | 0/<40 |
| NPDCS B % CR | 100/143 | 41/370 | 68/1290 | 83/872 | 76/322 | 100/206 |
| NPDCS C % TS | 0/<40 | 86/680 | 72/1098 | 20/183 | 47/545 | 44/236 |
| NPDCS C % CR | 100/523 | 14/113 | 28/426 | 80/750 | 53/606 | 56/299 |

TABLE 12

| NIH | | | | | | |
|---|---|---|---|---|---|---|
| Monovalent | | DENV3 | DENV3 | DENV1-3 | DENV1-3 | DENV1-3 |
| Serum | DENV1 | GII | GIII | EDI | EDII | EDIII |
| NIH2% TS | 0/<40 | 100/2782 | 78/365 | 0/<40 | 0/<40 | 98/5303 |
| NIH2% CR | 0/<40 | 0/<40 | 22/101 | 100/206 | 100/677 | 2/150 |
| NIH16% TS | 0/<40 | 97/954 | 90/226 | 64/227 | 0/<40 | 73/468 |
| NIH16% CR | 0/<40 | 3/33 | 10/25 | 36/126 | 100/101 | 27/174 |
| NIH47% TS | 0/<40 | 66/405 | 0/<40 | 18/93 | 0/<40 | 0/<40 |
| NIH47% CR | 0/<40 | 34/212 | 100/193 | 82/438 | 100/81 | 100/285 |
| NIH50% TS | 0/<40 | 53/6825 | 19/466 | 84/1182 | 100/149 | 100/642 |
| NIH50% CR | 100/94 | 47/5985 | 81/1986 | 16/226 | 0/<40 | 0/<40 |

The foregoing is illustrative of the present invention, and is not to be construed as limiting thereof. The invention is defined by the following claims, with equivalents of the claims to be included therein.

All publications, patent applications, patents and other references cited herein are incorporated by reference in their entireties for the teachings relevant to the sentence and/or paragraph in which the reference is present.

1. A chimeric dengue virus E glycoprotein comprising the amino acid sequence of any one of SEQ ID NOs: 1, 2, 3, 4, 5 and/or 6.

2. An isolated nucleic acid molecule encoding the E glycoprotein of claim 1.

3. The isolated nucleic acid molecule of claim 2, further encoding a chimeric prM glycoprotein.

4. The isolated nucleic acid molecule of claim 3, wherein the chimeric prM glycoprotein comprises the amino acid sequence of any one of SEQ ID NOs:7, 8, 9 and/or 10.

5-24. (canceled)

25. A flavivirus particle or virus like particle (VLP) comprising the E glycoprotein of any of claim 1.

26. The flavivirus particle or VLP of claim 25, further comprising a chimeric prM glycoprotein.

27. An isolated nucleic acid molecule encoding the flavivirus particle or VLP of claim 25.

28. A vector comprising the isolated nucleic acid molecule of claim 2.

29. A population of flavivirus particles comprising the flavivirus particle of claim 25.

30. A composition comprising the E glycoprotein of claim 1, in a pharmaceutically acceptable carrier.

31. A method of producing an immune response to a dengue virus in a subject, comprising administering to the subject an effective amount of the E glycoprotein of claim 1.

32. A method of treating a dengue virus infection in a subject, comprising administering to the subject an effective amount of the E glycoprotein of claim 1.

33. (canceled)

34. A method of protecting a subject from the effects of dengue virus infection, comprising administering to the subject an effective amount of the E glycoprotein of claim 1.

35-52. (canceled)
